(19) United States
(12) Patent Application Publication
(10) Pub. No.: US 2011/0206614 A1
(43) Pub. Date: Aug. 25, 2011

(54) SYSTEMIC INSTIGATION SYSTEMS TO STUDY TUMOR GROWTH OR METASTASIS

(75) Inventors: Sandra S. McAllister, Needham, MA (US); Robert A. Weinberg, Cambridge, MA (US)

(73) Assignee: Whitehead Institute for Biomedical Research

(21) Appl. No.: 12/745,221

(22) PCT Filed: Nov. 26, 2010
(86) PCT No.: PCT/US2008/085040

§ 371 Date: Feb. 25, 2011

Related U.S. Application Data
(60) Provisional application No. 61/004,704, filed on Nov. 28, 2007.
(60) Provisional application No. 61/003,916, filed on Nov. 23, 2007.
(60) Provisional application No. 61/060,774, filed on Jun. 11, 2008.

(51) Int. Cl. A61K 49/00; C12Q 1/02; G01N 33/53

(52) U.S. Cl. 424/9.2; 435/29; 424/9.1; 435/7.1; 436/501

(57) ABSTRACT

The present invention provides tumor instigation systems that can be used to characterize tumor growth and/or metastasis. In some embodiments, the present invention provides a method for studying tumor outgrowth or metastasis including the steps of: (a) providing an animal host; (b) introducing into the animal host one or more cells that instigate the growth of an otherwise indolent tumor; (c) introducing into the animal host a tumor; wherein the presence of the one or more cells or a progeny thereof in the animal host enhances the growth and/or metastasis of the tumor. Among other things, the inventive methods and systems in accordance with the present invention can be used to identify modulators and/or contributors of tumor growth or metastasis, as well as diagnostic and/or therapeutic agents for use in monitoring and/or modulating tumor growth and/or metastasis.

### RELATED APPLICATIONS

This application claims priority to U.S. Application No. 61/004,704, filed on Nov. 28, 2007, U.S. Application No. 61/063,916, filed on Feb. 6, 2008, and U.S. Application No, 61/060,774, filed on Jun. 11, 2008, the contents of all of which are hereby incorporated by reference in their entireties.

### BACKGROUND OF THE INVENTION

The tumor microenvironment has been the subject of intensive investigation in recent years. A tumor-supportive role for many of the host-derived stromal cells that form the tumor microenvironment has been demonstrated in a number of studies (Coussens and Werb, 2002; Murdoch et al., 2004; Pollard, 2004; Yang et al., 2004). For example, we and others demonstrated that stromal fibroblasts and myofibroblasts within the tumor environment facilitate tumor growth and progression (Bhowmick et al., 2004; Elenbaas and Weinberg, 2001; Olumi et al., 1999; Orimo et al., 2005; Tlsty, 2001).

Far less is known about the contribution to tumor growth of the systemic environment of a tumor-bearing host.

### SUMMARY OF THE INVENTION

The present invention provides tumor instigation systems that can be used to characterize tumor growth and/or metastasis. Among other things, inventive methods and systems in accordance with the present invention can be used to identify modulators and/or contributors of tumor growth or metastasis, as well as diagnostic and/or therapeutic agents for use in monitoring and/or modulating tumor growth and/or metastasis.

In some embodiments, the present invention provides a method for studying tumor outgrowth or metastasis including the steps of: (a) providing an animal host; (b) introducing into the animal host one or more cells that instigate the growth of an otherwise indolent tumor; (c) introducing into the animal host a tumor; wherein the presence of the one or more cells or a progeny thereof in the animal host enhances the growth and/or metastasis of the tumor. In some embodiments, the method of the present invention further includes a step of mixing the one or more cells with the tumor before introducing them into the animal host.

In some embodiments, the tumor is otherwise indolent in the animal host. In some embodiments, the tumor is a human tumor. In particular embodiments, the human tumor suitable for the present invention is an adenocarcinoma, a sarcoma, a melanoma, a Wilms' tumor, a lymphoma, or a benign tumor.

In some embodiments, the tumor is introduced in a form of individual cells. In other embodiments, the tumor is introduced as a tissue sample (e.g., a human tissue sample). In some embodiments, the tissue sample is a biopsy or surgical sample. In particular embodiments, the biopsy or surgical sample is derived from a tissue selected from the group consisting of breast, lymphoma, prostate, kidney, lung, liver, gastrointestinal tract, colon, testis, stomach, pancreas, thyroid, and brain. In certain embodiments, the biopsy or surgical sample is derived from a primary tumor. In certain embodiments, the biopsy or surgical sample is derived from a metastatic nodule.

In some embodiments, the one or more cells that instigate the growth of an otherwise indolent tumor are capable of proliferating in the animal host. In some embodiments, the one or more cells include tumor cells. In one embodiment, the tumor cells include human tumor cells. In some embodiments, the human tumor cells include individual cells derived from a human tumor biopsy or surgical sample. In other embodiments, the human tumor cells are introduced in a form of a biopsy or surgical sample. In some embodiments, the human tumor cells are selected from the group consisting of BPLER cells, MDA-MB-231 breast cancer cells and MCF7Ras breast cancer cells.

In other embodiments, the one or more cells capable of proliferating in the animal host include mouse tumor cells.

In certain embodiments, the one or more cells that instigate the growth of an otherwise indolent tumor include genetically modified cells. In one embodiment, the one or more cells are genetically modified to overexpress a protein. In certain embodiments, the one or more cells are genetically modified to overexpress a protein selected from a group consisting of a growth factor, a receptor, a cytokine, osteopontin, an oncogene, or a kinase. In another embodiment, the one or more cells are genetically modified to reduce a function of a gene. For example, the one or more cells are genetically modified to reduce the function of a tumor suppressor gene.

In some embodiments, the one or more cells and the tumor are introduced concurrently. In other embodiments, the one or more cells and the tumor are introduced sequentially.

In some embodiments, the one or more cells and the tumor are introduced at a same anatomical site. In other embodiments, the one or more cells and the tumor are introduced at distinct anatomical sites.

In some embodiments, the one or more cells or the tumor are introduced by subcutaneous injection, implantation, mammary fat pad injection or implantation, intravenous injection, bronchioalveolar lavage, intracardiac injection, intramuscular injection, kidney capsule implantation, mesenteric vein injection, intrafemoral injection, retroorbital injection.

In some embodiments, the animal host suitable for the present invention is a rodent. In particular embodiments, the animal host is a mouse. In certain embodiments, the mouse is an immunocompromised mouse. For example, the mouse may be selected from the group consisting of Nude, SCID, NOD-SCID, Rag1^{-/-}, and Rag2^{-/-}. In some embodiments, the mouse is a genetically engineered transgenic mouse.

In some embodiments, methods of the present invention further include administering a test agent to the animal host. In further embodiments, methods of the invention further include evaluating the ability of the test agent to inhibit the growth or metastasis of the tumor. In particular embodiments, test agents suitable for the invention include, but are not limited to, drugs, compounds, small molecules, antibodies or fragments thereof, cytokines, recombinant proteins, or nucleic acids.

In some embodiments, the present invention provides methods for studying tumor outgrowth or metastasis including steps of: (a) providing an activated animal host, wherein the activated animal host enhances the growth or metastasis of an indolent tumor compared to a corresponding un-activated animal host; and (b) introducing a tumor to the activated animal host.

In some embodiments, the tumor is a human tumor. In certain embodiments, the tumor is indolent in a corresponding un-activated animal host. In some embodiments, the activated animal host is a mouse.

In some embodiments, the activated animal host has an activated bone marrow. In particular embodiments, the activated bone marrow contains reduced primitive hematopoietic cells compared to a corresponding un-activated animal host. In certain embodiments, the activated bone marrow contains reduced primitive hematopoietic cells including Lin⁻/Sca⁺/cKit⁺ hematopoietic stem cells compared to a corresponding un-activated animal host. In certain embodiments, the activated bone marrow contains increased number of Sca⁺/cKit⁻ cells compared to a corresponding un-activated animal host. In certain embodiments, the activated bone marrow contains activated Sca⁺/cKit⁻ cells. As used herein, activated Sca⁺/cKit⁻ cells include those Sca⁺/cKit⁻ cells that are mobilized, in particular, those circulated into blood system. In some embodiments, a blood sample from the activated animal host contains mobilized Sca⁺/cKit⁻ cells that were activated in the bone marrow. In some embodiments, responding tumors on the activated host recruit those bone marrow-derived, mobilized Sca⁺/cKit⁻ cells into their stroma. In certain embodiments, the activated animal host has an increased osteopontin level compared to a corresponding un-activated animal host.

In some embodiments, the activated animal host bears one or more tumor cells. In some embodiments, the activated animal host bears one or more tumor cells that are human tumor cells. In particular, the human tumor cells are selected from the group consisting of BPLER cells, MDA-MB-231 breast cancer cells and MCF7Ras breast cancer cells.

In some embodiments, the activated animal host bears an implanted surgical or biopsy tumor sample. In some embodiments, the activated animal host bears an implanted surgical or biopsy tumor sample derived from a human patient.

In other embodiments, the activated animal host is a transgenic mouse bearing one or more spontaneously-arising tumors.

In certain embodiments, the activated animal host bears a surgical wound.

In certain embodiments, the activated animal host has an increased level of one or more cytokines (e.g., autocrine, paracrine or endocrine) or chemokines. In one embodiment, the activated animal host is a mouse that has an increased level of one or more cytokines (e.g., autocrine, paracrine or endocrine). In particular embodiments, the activated animal host is a mouse bearing an implanted slow-release pellet secreting one or more cytokines. In other embodiments, the activated animal host is a transgenic mouse over-expressing one or more cytokines. As used herein, “cytokines” refer to kinetic proteins and peptides that are used in organisms as signaling compounds. In particular, cytokines includes proteins and peptides that are used to allow one cell to communicate with another. Typically, a cytokine can be an autocrine (a cytokine that acts on the cell that secretes it), a paracrine (a cytokine whose action is restricted to the immediate vicinity of a cytokine's secretion), or an endocrine (a cytokine that diffuses to distant regions of the body (e.g., carried by blood or plasma) to affect different tissues). Generally, the cytokine family includes smaller water-soluble proteins and glycoproteins (proteins with an added sugar chain) with a mass typically between 8 and 30 kDa.

In some embodiments, the present invention provides methods for evaluating a human surgical tumor specimen including steps of: (a) providing an animal host that instigates the growth of an otherwise indolent human tumor; (b) introducing a human surgical tumor specimen into the animal host; (c) evaluating the growth of the human surgical tumor specimen on the animal host. In some embodiments, the evaluation of the growth of the human surgical tumor specimen is based on a comparison to the growth of a control tumor on the animal host. In some embodiments, inventive methods of the invention further include a step of diagnosing the human tumor based on the evaluation result from step (c). In some embodiments, the diagnosis of the human tumor includes determining the risk of metastasis, fast-growing phenotype or drug resistance. In other embodiments, the diagnosis of the human tumor includes providing prognosis of the human tumor. In further embodiments, the diagnosis of the human tumor includes determining a treatment for the human tumor, such as, for example, a chemotherapy, radioactive therapy, or an anti-osteopontin therapy.

In some embodiments, the present invention provides methods for evaluating a human surgical tumor specimen including steps of: (a) mixing a human surgical tumor specimen with one or more activated bone marrow cells, wherein the one or more activated bone marrow cells instigate the growth of the human surgical sample on an animal host; (b) introducing the mixture of step (a) into an animal host; and (c) evaluating the growth of the human surgical tumor specimen on the animal host. In some embodiments, inventive methods further include a step of diagnosing the human tumor based on the evaluation result from step (c). In other embodiments, activated bone marrow cells suitable for the present invention are derived from a mouse bearing a tumor that enhances the growth of an otherwise indolent human tumor.

In some embodiments, the present invention provides methods for evaluating the instigating ability of tumors in animal hosts including steps of: (a) providing an animal host; (b) introducing one or more cells derived from the tumor to be evaluated into the animal host; (c) introducing an otherwise indolent tumor into the animal host; and (d) evaluating the growth or metastasis of the otherwise indolent tumor to determine the instigating ability of the tumor. In some embodiments, tumors to be evaluated are human tumors. In certain embodiments, tumors to be evaluated are surgical or biopsy samples taken from human patients. In some embodiments, inventive methods further include a step of diagnosing the tumor based on the evaluation result from step (d).

In some embodiments, the present invention provides methods for determining the risk of tumor outgrowth or metastasis for a patient including steps of: (a) providing an animal host; (b) introducing one or more cells derived from the patient bearing the tumor into the animal host; (c) introducing an otherwise indolent tumor into the animal host; and (d) evaluating the growth of the otherwise indolent tumor to determine the risk of tumor outgrowth or metastasis for the patient. In some embodiments, one or more cells derived from the tumor are introduced into the animal host. In other embodiments, one or more cells derived from the bone marrow of the patient are introduced into the animal host. In certain embodiments, inventive methods of the invention further include a step of diagnosing the tumor based on the evaluation result from step (d). In certain embodiments, the diagnosis of the human tumor includes providing prognosis of the tumor. In certain embodiments, the diagnosis of the tumor includes determining a treatment for the tumor. Suitable treatments include, but are not limited to, chemotherapy, radioactive therapy, or anti-osteopontin therapy.

In some embodiments, the present invention provides methods for evaluating the ability of an agent to inhibit tumor outgrowth or metastasis including steps of: (a) providing an animal host that instigates the growth or metastasis of an otherwise indolent tumor; (b) introducing a tumor into the animal host; (c) administering an agent to the animal host bearing the tumor; and (d) evaluating the ability of the agent to inhibit the growth of the tumor. In some embodiments, inventive methods further includes a step of comparing the evaluation result from step (d) to a control to determine whether the agent is capable of inhibiting the tumor outgrowth or metastasis. In some embodiments, the animal host bears a first tumor that is capable of enhancing the growth of the otherwise indolent tumor. In certain embodiments, agents evaluated in accordance with the present invention include, but are not limited to, drugs, compounds, small molecules, antibodies or fragments thereof, cytokines, or recombinant proteins.

The invention also provides modulators (e.g., inhibitors) of tumor outgrowth or metastasis identified by various methods described herein.

In some embodiments, the present invention provides methods for reducing the risk of tumor metastasis by inhibiting an activity of osteopontin. In some embodiments, the activity of osteopontin is inhibited by an interfering RNA. In some embodiments, the interfering RNA suppresses the expression of osteopontin. In other embodiments, the interfering RNA suppresses the expression of an osteopontin receptor. In certain embodiments, the interfering RNA is selected from siRNA, shRNA or miRNA. In one particular embodiment, the interfering RNA includes the sequence of CCGGCCACAAGCAGTCCAGATTATACTCGAGTATAATCTGGACTGCTTGTGGTTTTT (SEQ ID NO: 3). In another embodiment, the interfering RNA includes the sequence of CCGGCCGAGGTGATAGTGTGGTTTACTCGAGTAAACCACACTATCACCTCGGTTTTT (SEQ ID NO: 4).

In some embodiments, the activity of osteopontin is inhibited by an antibody. In some embodiments, the antibody binds to osteopontin. In other embodiments, the antibody binds to an osteopontin receptor. In certain embodiments, the antibody is a monoclonal antibody. In certain embodiments, the antibody is a single chain antibody. In certain embodiments, the antibody is an antibody fragment. In certain embodiments, the antibody is a humanized antibody.

In some embodiments, the present invention provides mice that instigate or are capable of instigating growth of an otherwise indolent human tumor. In some embodiments, mice in accordance with the present invention have activated bone marrow. In certain embodiments, the activated bone marrow contains reduced primitive hematopoietic cells. In certain embodiments, the bone marrow contains activated Sca⁺/cKit⁻ cells. In certain embodiments, mice in accordance with the present invention bear a human tumor that enhances the growth of an otherwise indolent human tumor. In certain embodiments, mice in accordance with the present invention bear a human tumor selected from the group consisting of BPLER tumor, MDA-MB-231 breast cancer and MCF7Ras breast cancer cells. In certain embodiments, mice in accordance with the present invention have an increased level of one or more cytokines (e.g., paracrine or endocrine). In particular embodiments, mice in accordance with the present invention bear one or more implanted slow-release pellets secreting one or more cytokines. In particular embodiments, mice in accordance with the present invention bear one or more cells over-expressing one or more cytokines. In other embodiments, mice in accordance with the present invention bear are transgenic mice over-expressing one or more cytokines.

In some embodiments, the present invention also provides methods for identifying modulators of tumor instigation, outgrowth or metastasis. For example, inventive methods may include steps of: (a) providing a sample obtained from an animal host that instigates the growth of an otherwise indolent tumor; (b) providing a control sample; and (c) comparing the sample of (a) with the control sample of (b) so as to identify one or more components that differ between the samples, wherein a component that differs between the two samples is identified as a candidate modulator of systemic tumor instigation. In some embodiments, the animal host of step (a) bears an instigator. In some embodiments, the instigator is selected from the group consisting of one or more cells (e.g., human cells), an implanted tumor sample, a spontaneously-arising tumor, a surgical wound, and combinations thereof. In some embodiments, the instigator comprises one or more cells that are experimentally generated tumor cells. In some embodiments, the instigator comprises one or more cells derived from a naturally occurring tumor. In some embodiments, the instigator comprises one or more cells that secrete OPN.

In some embodiments, inventive methods include steps of: (a) providing a sample obtained from an animal host bearing one or more cells or compositions secreting or releasing OPN; (b) providing a control sample; and (c) comparing the sample of (a) with the control sample of (b) so as to identify one or more components that differ between the samples, wherein a component that differs between the two samples is identified as a candidate modulator of systemic tumor instigation.

The samples can be compared by various methods, including but not limited to expression profiling methods and other methods described herein and known in the art. Candidate modulators identified in accordance with the present invention can be further tested for their ability to enhance tumor growth and/or metastasis using tumor instigation system described herein or using methods known in the art. Candidate modulators that determined to be contributors to tumor growth or metastasis may be utilized as targets for further development of anti-tumor therapy. For example, contributors to tumor growth or metastasis can be used to identify therapeutic agents that modulate (e.g., inhibit) the expression and/or activity of such contributors. In some embodiments, contributors to or modulators of tumor growth or metastasis identified in accordance with the present invention may be used to further identify proteins (e.g., receptors, cell surface markers) or cell types to which the modulators or contributors bind. Such receptors, cell surface markers, or cell types identified can be used as targets for development of anti-tumor therapy.

In some embodiments, the present invention provides methods of identifying cell types that contribute to systemic instigation including steps of: (a) providing a sample obtained from an animal host bearing one or more cells that instigate the growth of an otherwise indolent tumor; (b) measuring populations of one or more cell types in the sample; (c) identifying at least one cell type whose population is enriched in the sample as compared to a control.

In some embodiments, the present invention provides methods of testing a candidate anti-tumor agent including steps of: (a) administering a candidate anti-tumor agent to an animal host bearing one or more cells that instigate the growth of an otherwise indolent tumor; and (b) determining whether the candidate anti-tumor agent increases the population of a cell type that enhances outgrowth or metastasis of a tumor or inhibits migration of said cell type to a tumor.

In some embodiments, the present invention provides methods of identifying a candidate biomarker indicative of tumor metastasis or outgrowth including steps of: (a) providing a sample obtained from a subject bearing one or more cells that enhance the growth of a remote tumor; (b) measuring populations of one or more cell types in the sample; (c) identifying at least one cell type whose population is enriched or reduced in the sample as compared to a control, wherein the at least one cell type is a candidate cell marker indicative of tumor metastasis or outgrowth.

In some embodiments, the present invention also provides methods of prognosis of a cancer patient including steps of: (a) providing a sample obtained from a cancer patient; (b) measuring the population of a cell marker indicative of tumor metastasis or outgrowth; and (c) determining the prognosis of the cancer patient based on the result from step (b).

The present invention also provides diagnostic or therapeutic methods using modulators, biomarkers or therapeutic agents identified in accordance with the invention.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### DESCRIPTION OF THE DRAWINGS

The drawings are for illustration purposes only, not for limitation.

FIG. 1. Exemplary data illustrating that primary human breast carcinomas facilitate growth of distant indolent tumors. (A) Scheme of bilateral implantation system. Indolently growing HMLER-HR transformed cells (Responders) are implanted subcutaneously into one flank of host mice and either Matrigel control or vigorously growing tumor cell lines (Instigators) are implanted into the contralateral flank. (B) In vivo growth kinetics of responder cells when implanted contralaterally to either Matrigel (triangles) or BPLER instigators (squares); n=5 per group. (C) Final mass of responders from (B) 9 weeks after implantation opposite either Matrigel or instigating BPLER tumors. Incidence of tumor formation is shown above data bars; data include injections not resulting in responding tumor growth. (D) In vivo growth kinetics of responders when injected 30 days after implantation of either Matrigel plugs (triangles) or instigating BPLER tumor cells (squared); n=5 per group. (E) Average final mass of responding tumors from (D) recovered opposite either Matrigel or BPLER instigators; incidence of responder tumor formation is indicated above data bars. (F) Final mass of responding tumors recovered opposite Matrigel or indicated tumor cell lines 9 weeks after implantation. Incidence of responding tumor formation is indicated above bars; data include mice from three separate experiments. Mass of responders recovered opposite Matrigel is significantly different from those opposite 231 (p=0.031) and BPLER (p=0.039).

FIG. 2. Exemplary data illustrating that bone marrow-derived cells are incorporated into responding tumor stroma. (A) Indicated tumor cells were injected into mice that had previously been engrafted with GFP BMCs. (B) Whole mount fluorescence photomicrographs to visualize GFP BMCs recruited to indicated tumors and control tissues 4 weeks after tumor cell injections; scale bar=2 mm. Average mass of all tissues is indicated; n=10 mice per group. (C) Average contribution of GFP cells as a percentage of total cells (flow cytometric analysis); n=7 tumors/tissues per group. Responding tumors opposite BPLER instigators incorporated significantly more GFP BMCs than those opposite Matrigel (p=0.039) and PC3 noninstigating tumors (p=0.042); responders were not statistically different from their contralateral BPLER instigators; instigating BPLER tumors were not statistically different from noninstigating PC3 tumors.

FIG. 3. Exemplary data illustrating that instigators functionally activate the bone marrow in a tumor-supportive fashion. (A) Experimental scheme for implantation of BMCs/responding tumor cell admixtures. (B) Average mass of resulting tumors 12 weeks after implantation of mixtures of responder cells with indicated BMCs. Tumor incidence is indicated above bars; data represent mean of four separate experiments. p=0.012 comparing BM-I with responders alone; p=0.015 comparing BM-I with BM-C; p=0.001 comparing BM-I with BM-NI. (C) Hematoxylin and Eosin stain to visualize histopathology of resulting tumors/tissues; scale bar=400 μm.

FIG. 4. Exemplary data illustrating that instigating tumors mediate reduction of lin⁻/sca1⁺/cKit⁺ cells in the marrow and an elevation of Sca1⁺ cells in responding tumors. (A) Flow cytometric analysis of LSK cells in the bone marrow of various tumor-bearing mice; n=4 per group. (B) Quantification of Sca1⁺/cKit⁺ and Sca1⁺/cKit⁻ cells as percentage of total GFP cells in responding tumors recovered opposite instigating BPLER tumors (black) or noninstigating PC3 tumors (gray); n=7 per group. (C) Merged photomicrographs of indicated responding tumors stained for GFP (green), Sca1 (red), and cell nuclei (blue); GFP⁺/Sca1⁺ cells appear yellow (arrows); scale bar=25 μm.

FIG. 5. Exemplary data illustrating that tumor-derived osteopontin is important for systemic instigation. (A) Concentration of hOPN in plasma of mice 9 weeks after injection of indicated cells; n=14 for responder group; n=5 for all other groups. Plasma OPN was significantly elevated in mice bearing BPLER (p=0.02) and 231 (p=0.01) tumors compared with plasma from mice injected only with responder cells. (B) The left shows in vivo growth kinetics of instigating MDA-MB-231 (parental), control 231 cells expressing shRNA against Luciferase (shLucif), and 231 derivatives expressing shRNAs against hOPN; n=9 for parental group; n=5 for all other groups. The right shows in vivo growth kinetics of the responding cells injected opposite the indicated tumor cell lines. (C) Flow cytometric analysis of LSK cells in the bone marrow of mice with indicated bilateral tumors; n=4 per group. (D) Admixtures of responder cells with BMCs from mice bearing 231 instigators yielded tumors that were significantly larger than those resulting from admixtures of BMCs from mice bearing shOPN 1 tumors (p=0.030), shOPN 5 tumors (p=0.047), or responder cells alone (p=0.026); n=4 per group.

FIG. 6. Exemplary data illustrating that instigating tumors promote outgrowth of disseminated lung metastases. (A) Model of metastatic systemic instigation: GFP instigating tumor cells (1° tumor) or Matrigel control are subcutaneously (s.c.) injected into both flanks of host mice while weakly metastatic GFP⁺ tumor cells are injected intravenously (i.v.). (B) Average numbers of micrometastatic lung foci after concurrent injection of weakly metastatic 231 cells i.v. and either instigating BPLER tumors or Matrigel control plugs s.c.; n=4 mice per group. (C) Whole-mount fluorescent photomicrographs of 231+GFP responder lung foci in mice bearing either Matrigel control plugs (a, b) or GFP instigating 1° tumors (c, d); scale bar=0.5 mm. (D) Immunohistochemical staining of lung sections for GFP⁺ responder cells (red); nuclei stain, blue; scale bar=200 μm. (E) For (E) and (F), mice received s.c. injections of either Matrigel, GFP⁻ parental 231 cells or GFP⁻ shOPN 231 cells and i.v. injection of weakly metastatic 231+GFP cells. Graph depicts numbers of micrometastatic 231+GFP lung foci from each mouse after 4 weeks; lines denote average number of foci per group; average 1° tumor burden is indicated. Whole-mount fluorescent photomicrographs depict micrometastatic foci (arrows); scale bar=0.5 mm. (F) Numbers of macrometastatic 231+GFP foci counted by eye in the lungs of each mouse. Whole-mount images of lungs are shown with corresponding fluorescent images; scale bar=2 mm.

FIG. 7. Exemplary data illustrating that human colon tumor surgical specimen responds to systemic instigation. (A) The left shows human colon tumor segments (“colon responder”; black line) dissected from a single patient's surgical specimen and implanted opposite Matrigel plugs (gray line). The center shows in vivo growth kinetics of colon responder tumor segments (black line) implanted opposite instigating BPLER breast carcinoma cells (gray line). The right shows that neither the human colon tumor segments (black line) nor the HMLER-HR breast responder cells (gray line) are able to grow when implanted opposite one another. n=3 mice per group. (B) Hematoxylin and Eosin stain of a colon responder recovered opposite a breast instigator, scale bar=50 μm. (C) Staining for Ki67 (brown) to reveal proliferating responding colon tumor cells implanted opposite BPLER breast instigators, scale bar=50 μm. (D) Model of Systemic Instigation: Instigating tumors secrete osteopontin (OPN), which perturbs primitive hematopoietic cells in the host bone marrow; cells in the bone marrow are functionally activated prior to mobilization into the circulation; release of activated bone marrow-derived cells into the circulation and their subsequent incorporation into distant responding tumor stroma serve to foster outgrowth of the once-indolent cells into growing adenocarcinomas.

FIG. 8. Exemplary data illustrating that instigating tumors facilitate responder growth without metastasizing to sites of responder injection. (A) Experimental protocol and graph representing in vivo growth kinetics of instigating BPLER tumors (squares) and responding HMLER-HR tumors (diamonds) when all cells were implanted simultaneously. Data represent mean tumor diameter; error bars represent s.e.m. These data correspond to data represented in FIG. 1B,C. (B) Experimental protocol and graph showing in vivo growth kinetics when the HMLER-HR cells (squares) were injected 30 d after implantation of the instigating BPLER tumors (diamonds). These data correspond to data represented in FIG. 1D,E. (C) Hematoxylin and eosin staining of responding tumors to reveal cell nuclei (purple); asterisk denotes necrosis, which was only observed in the responders recovered opposite Matrigel; scale bar=200 μm. (D) Tumor sections stained for GFP (dark red) confirmed that GFP+BPLER instigating cells had not metastasized to the contralateral sites of GFPnegative responder cell injection. Cell nuclei are counterstained with hematoxylin (blue); scale bar=200 μm. (E) Immunoperoxidase stain of a responding tumor implanted opposite MDA-MB-231 cells in order to visualize the LgT Antigen (dark red), which is expressed only by HMLER-HR responder cells; nuclei are counterstained with hematoxylin (blue).

FIG. 9. Exemplary data illustrating equivalent engraftment of GFP⁺BMCs into mice subsequently used in experiments. (A) BMCs from Rag1−/−EGFPTg donor mice were transplanted into recipient immunocompromised mice. Graph represents flow cytometric analysis of GFP+ donor BMCs in the blood of recipient mice at the initiation of the experiment (“Bloodi”) and in blood and bone marrow upon completion of the experiment (“Bloodf” and “BMf”). Data represent mean; ±s.e.m.; n=9 per group. (B) GFP+BMCs recruited to a responding tumor opposite Matrigel (left) and a responding tumor with its contralateral instigating BPLER tumor (right) 9 week after tumor cell injections. Average contribution of GFP+ cells as a percentage of total cells is indicated; (n=4); p=0.03 comparing responders to control; p=0.04 comparing instigators to control; responders are not statistically different from instigators. (C) GFP+BMCs (green) are incorporated into the stroma of these 9 week responding and instigating tumors; DAPI stain (blue) shows cell nuclei; scale bar=50 μm.

FIG. 10. Exemplary data illustrating that admixed bone marrow from tumor-bearing mice does not contain tumor cells and is maintained in growing responding tumors. (A) Precise numbers of GFP+BPLER cells from culture were added to 2×10⁴ normal BMCs and the ability to detect GFP+ cells in each sample was analyzed by flow cytometry. Numbers of GFP+ tumor cells were quantified and plotted against the number of GFP+ tumor cells originally added to each bone marrow sample to create a standard curve. GFP+ tumor cells could be reliably detected at a lower limit of 5 tumor cells per 2×10⁴ BMCs. (B) Flow cytometric analysis of BMC samples used in admixing experiments (see Experimental Procedures). BMCs harvested from mice bearing GFP+BPLER instigating tumors were analyzed for the presence of GFP+ tumor cells by flow cytometry. No GFP+ tumor cells were observed in the bone marrow samples by this method, thus indicating that fewer than 5 tumor cells per 2×10⁴ BMCs could possibly be present in the donor bone marrow samples that were used to admix with responder cells in our experiments. Since 7.5×10⁵ total bone marrow cells were used for the admixing experiments, the theoretical upper limit to the number of instigating tumor cells present in these BMC samples would be 188 tumor cells in total. (C) GFP+ BMCs from Rag1−/−xEGFPTg mice bearing GFP-negative BPLER instigating tumors were admixed to responder cells prior to injection into experimental mice and tumors were examined 9 weeks later. (a) Whole mount fluorescence microscopy to visualize GFP+BMCs in a responding tumor. (b) Immunofluorescence analysis to visualize GFP+BMCs (green) and nuclei (blue) in a responding tumor tissue section (10× objective). (c) Immunofluorescence analysis to visualize LgT Ag-positive responding tumor cells (red), GFP+BMCs (green) and cell nuclei by DAPI (blue); the merged image yields purple where nuclear LgT Ag overlays with DAPI (40× objective). (D) Mass of resulting responding tumors after mixture of responder cells with BMCs from mice bearing small instigating tumors (<60 mg) compared with BMC from mice bearing only Matrigel plugs.

FIG. 11. Exemplary data illustrating LSK cells in bone marrow of instigator-bearing mice; mOPN levels in plasma of mice. (A) Flow cytometric analysis of BMCs harvested from BPLER instigator-bearing mice after 8-10 weeks of tumor growth showed that Nude mice (left; n=15 per group) and NOD-SCID mice (right; n=26) had a significant reduction in marrow LSK cells compared with respective Matrigel control mice. (B) Photomicrograph of a responding tumor that had grown opposite a BPLER instigating tumor for 8 weeks showing that Sca1+ cells (red) are incorporated into the growing tumor stroma. Cell nuclei are counterstained with DAPI (blue); scale bar=50 μm. (C) Relative concentration of mouse OPN (mOPN) in plasma of various tumor-bearing mice. None of the indicated tumor types had a significant effect on levels of circulating mOPN (n=10 per group for Matrigel and BPLER; n=5 per group for all other groups). (D) Concentration of hOPN in plasma from 2 human breast cancer patients with metastatic disease (BrCa Met) with respect to cancer-free subjects (BrCa Free). The concentration of hOPN in the plasma of each cancer-free subject is significantly different from both BrCa Met 1 (p=0.037 and p=0.032, respectively) and BrCa Met 2 (p=0.024 and p=0.018, respectively). Samples were run in triplicate.

FIG. 12. Exemplary data illustrating 80 human cytokines tested in plasma from control and tumor-bearing mice. Plasma from groups of mice bearing either Matrigel control plugs, instigating human tumors or non-instigating human tumors was screened for relative levels of various human cytokines by cytokine antibody array (RayBiotech™). Additionally, plasma concentrations of human osteopontin (hOPN) and human VEGF were tested by ELISA. Of these cytokines, only hOPN was significantly elevated in the plasma of instigator-bearing mice relative to that of noninstigator-bearing mice.

FIG. 13. Exemplary data illustrating verification of OPN suppression in MDA-MB-231 cells and maintenance of suppression in vivo. (A) Western blot of cell lysates prepared from instigating MDA-MB-231 cells (parental), MDA-MB-231 derivatives expressing shRNAs against human osteopontin (hOPN), and HMLER-HR responder cells as probed for hOPN protein; bands of expected sizes are noted. Also shown is anti-β-actin loading control. (B) ELISA to quantify concentration of hOPN secreted by various cell types into culture media during a 24 h period. Data represent mean values for triplicate samples ±s.e.m. (C) In vitro growth curves of MDA-MB-231 cells (parental) and the shOPN derivative cell lines. Data represent mean values of 2 separate experiments with each sample run in triplicate ±s.e.m (D) Photomicrographs of cultured cells to indicate morphology. Cells expressing shOPN#1, shOPN#5, and control shRNA against the irrelevant protein, luciferase, had morphology identical to that of the parental MDA-MB-231 cells; cells expressing shOPN#3 and shOPN#4 displayed altered morphology (not shown) and were not used in subsequent experiments. (E) hOPN was quantified by Q-PCR from mRNA prepared from indicated tumors that had grown as xenografts for 90d. Whether these tumors instigated growth of the contralateral responder cells is indicated. (F) Whole mount immunofluorescent photomicrographs of responding tumor masses grown opposite 231 parental and shOPN#5 tumors. Average percent contribution of GFP+BMCs to total cells in the tumor/tissue masses is indicated. Images were taken under identical exposure and gain.

FIG. 14. Exemplary data illustrating that ectopic expression of hOPN may not be sufficient to turn non-instigating PC3 cells into instigators. (A) Levels of hOPN secreted into culture media by the indicated tumor cells was quantified by ELISA (R&D). Cells tested were: non-instigating PC3 parental cell line, PC3 cells ectopically expressing hOPN(PC3OPN), instigating MDA-MB-231 cells and the derivative 231 cells expressing shRNA to OPN (shOPN#5). (B) Photomicrographs of cultured PC3 parental cells and PC3OPN cells. Ectopic expression of hOPN in these cells altered their morphology from the typical cobblestone phenotype (PC3 parental) to that of mesenchymal-like phenotype (PC3OPN). (C) Measurement of in vitro growth revealed that the PC3OPN cells (circles) grew with nearly identical kinetics as the parental PC3 cells (squares). Data represent results of triplicate analysis in each of 2 separate experiments ±s.e.m. (D) Concentration of hOPN in plasma of mice that were implanted with responding tumor cells contralateral to the indicated cell lines or Matrigel control. Data indicate that hOPN is circulating in the plasma of mice that had been implanted with PC3OPN cells contralaterally to responding cells. n=5 per group. (E) Left: In vivo growth kinetics of the non-instigating parental PC3 tumor cell xenografts (gray) and the PC3OPN derivatives (black). Right: In vivo growth kinetics of the responding HMLER-HR cells injected opposite the PC3 parental non-instigators (gray) or the PC3OPN derivatives (black); n=6 per group.

FIG. 15. Exemplary data illustrating systemic instigation of lung metastases. (A) Weakly metastatic GFP+MDA-MB-231 cells were injected i.v. while either Matrigel control or GFP-negative instigating BPLER cells were injected subcutaneously into host mice (see experimental scheme, FIG. 6A). Whole mount immunoflourescent photomicrographs show GFP+metastatic foci in selected lungs from mice as indicated; scale bar=5 mm. (B) Immunohistochemical staining of serial lung sections from a mouse in which subcutaneous instigating tumors induced the outgrowth of GFP+ metastatic foci. Staining for GFP (red, left image) and lack of staining for the LgT antigen (right image), showed that this metastatic outgrowth was formed exclusively from the GFP+ responder cells that were injected i.v., and not from unexpected metastasis of the GFP-negative instigating cells that were injected subcutaneously, which express the LgT antigen; scale bar=50 μm. (C) (Left) Merged fluorescent photomicrographs when lung tissue sections were stained for 231+GFP responder cells (green), Sca1+ stromal cells (red) and counterstained to label cell nuclei (blue); scale bar=25 μm. (Right) Fluorescent photomicrograph of a lung section from a normal mouse showing that the only Sca1+ cells (red) were associated with normal pulmonary vasculature (PV). Cell nuclei are counterstained with DAPI (blue).

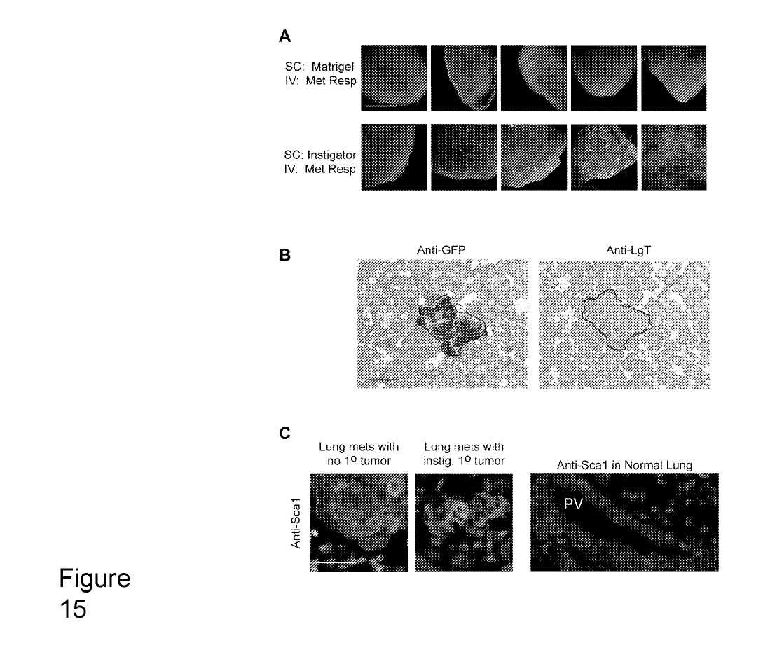

FIG. 16. Exemplary histopathology of primary human colon tumor surgical specimen. (A) Protocol for in vivo passage of human tumor surgical samples in SCID mice and selection of sample to be used as a responder in the bilateral instigation experiments. (B) Left: Two different photomicrographs showing H & E staining of the same primary human colon carcinoma as a fresh surgical specimen. This tumor was described as a moderately differentiated invasive colon adenocarcinoma with mutiple pericolonic positive lymph nodes. Right: Photomicrographs showing H & E staining of the human colon tumor segments (from tumor depicted in left panels) that were passaged twice as xenografts in SCID mice. Shown are representative tumor xenografts from 2 different mice. This sample was selected as a potential responder due to its slow growth kinetics in the SCID mice (˜4.5 months to reach 1 cm in diameter), and histopathology involving widespread necrosis (asterisks). (C) Staining of a serial section showed that Sca1+ cells (red) are present in stromal areas adjacent to these proliferating tumor cells only when contralateral instigating BPLER tumors are present; scale bar=50 μm.

FIG. 17. Exemplary data illustrating that stromal desmoplasia arises as a result of systemic instigation. (A) Systemic instigation injection protocol. Indolently growing Responder cells were implanted subcutaneously into one flank of host mice and either Matrigel control (left) or vigorously growing Instigating cells (right) were implanted into the contralateral flank. (B) Growth kinetics of Instigating and Responding tumors. Data points represent average tumor diameter and error bars represent s.e.m. (C) Immunohistochemical analysis of resulting tumors. Tissues were stained for SMA+ cells (red, top panels) or with Masson's trichrome to visualize collagen deposition (blue, bottom panels). Responding cell masses recovered from sites contralateral to Matrigel plugs displayed very little collagen deposition or αSMA expression; in contrast, αSMA-positive cells and collagen were distributed widely and uniformly throughout the responding tumors that were implanted contralaterally to instigating tumors. (D) Experimental protocol for implantation of admixtures of bone marrow cells (BMCs) with responding tumor cells. (E) The bone marrow of mice bearing instigating tumors phenocopies the effects of systemic instigation with respect to stromal desmoplasia. SMA+ cells (red) were only associated with blood vessels in tumors that formed after admixture of control BMCs with responder cells (top panel), while SMA+ cells were distributed widely and uniformly throughout tumors that arose as a result of admixture of instigator-bearing BMCs with responder cells (bottom panel).

FIG. 18. Exemplary data illustrating that the majority of the SMA+ cells within the instigated tumors are myofibroblasts, a hallmark of stromal desmoplasia. (A) Instigating BPLER tumors form with a reactive stroma, as indicated by the formation of a myofibroblast-rich stroma (SMA+ stain, red). (B) Responding tumor tissues stained with an antibody against smooth muscle actin (αSMA; red, top panels) or an antibody against the mouse endothelial cell antigen (MEDA32; brown, bottom panels). αSMA+ cells in the resulting resonding tumors overlapped with staining for endothelial cells only to a minimal extent, suggesting that the majority of αSMA+ cells in these instigated tumors were myofibroblasts rather than pericytes. (C) Similar to our previous results, admixed control BM from the Matrigel-bearing mice did not significantly enhance responding tumor size when compared with implantation of responder cells alone; admixture of bone marrow cells from instigator-bearing animals increased the incidence of tumor formation and enhanced the size of those tumors that did form, when compared with the control BMC mixtures. (D) In the tumors that arose as a result of admixture of responder cells with BMCs from instigator-bearing mice, αSMA (red, right panel) stained not only pericytes, but also the widely dispersed, abundant myofibroblasts, as indicated by a serial section stained for MECA32 (brown, left panel).

FIG. 19. Exemplary data illustrating that Sca+ cells from instigator-bearing mice phenocopy systemic instigation. (A) Representative density plots show bone marrow cell populations that were collected by fluorescence activated cell sorting (FACS) of bone marrow that was harvested from instigator-bearing mice. Table shows the number of each bone marrow cell population that was admixed with responder cells prior to injections into host mice. (B) Size of resulting tumors, relative to injection of responder cells alone, after mixing responder cells with indicated bone marrow cell populations. Numbers above bars indicate incidence of tumor formation. (C) Histopathology of resulting tumors after mixture of responder cells with indicated bone marrow cell populations prior to injection into host mice. Collagen deposition (blue) was visualized by staining with Masson's Trichrome (indicated) and smooth muscle actin (red) was visualized by immunohistochemical analysis. Note that tumors resulting from admixture of responder cells with Sca+BMCs most closely resembeled the histopathology, including stromal desmoplasia, of systemically instigated tumors.

FIG. 20. Exemplary data illustrating that Sca+/cKit− cells from instigator-bearing animals are unique in their tumor promoting function. (A) Density plots showing the segregation of Sca+bone marrow cells, prepared from instigator-bearing and control donor mice, into Sca+/cKit+ and Sca+/cKit− subpopulations by FACS. (B) Size of resulting tumors, relative to injection of responder cells alone, after mixing responder cells with indicated bone marrow cell populations. Only the Sca+/cKit− bone marrow subpopulation from instigator-bearing mice was able to functionally support responding tumor growth. (C) Histopathology of resulting tumors after mixture of responder cells with indicated bone marrow cell populations prior to injection into host mice. Collagen deposition (blue) was visualized by staining with Masson's Trichrome (left panels) and smooth muscle actin (red; right panels) was visualized by immunohistochemical analysis. Note that the Sca+/cKit− BMCs from instigator-bearing mice (middle panels) induced formation of a desmoplastic stroma while the same population from control mice (bottom panels) did not. (D) Sca+ cells (green) are retained in the stroma of responding tumors that were instigated by admixture of Sca+/cKit− cells from instigator-bearing donor mice. Cell nuclei were counterstained with DAPI (blue). (E) Characterization of some common cell surface antigens expressed (or absent) on the Sca+/cKit− population of instigator-bearing or control mice. There were no significant differences in the representation of these cell surface markers when comparing the marrows of indicated mice.

FIG. 21. Exemplary data illustrating in vivo growth kinetics of Her2-positive SKBR3 cells when implanted contralaterally to either Matrigel (blue) or BPLER breast carcinoma instigators (red); n=3 per group. Error bars represent s.e.m.

FIG. 22. Exemplary date illustrating in vivo growth kinetics of the instigating MDA-MB-231 breast carcinomas (black) and the contralateral Her2-positive SKBR3 responding tumors (red; as shown in FIG. 21). n=3 per group; error bars represent s.e.m.

FIG. 23. Exemplary data illustrating in vivo growth kinetics of the non-instigating Her2-positive BT474 breast carcinomas (green) and the contralateral HMLER-HR cells (“HR”; blue). Note that despite the vigorous growth of the BT474 tumors, the contralateral responding cells failed to form tumors. n=3 per group; error bars represent s.e.m.

### DEFINITIONS

Activated bone marrow: As used herein, the term “activated bone marrow” includes any bone marrows or bone marrow cells that induce, stimulate, or enhance tumor outgrowth or metastasis.

Agent: As used herein, the term “agent” (also referred to as “test agent” or “candidate agent”) refers to any compound or composition that can be tested as a potential modulator. Examples of agents that can be used include, but are not limited to, small molecules, antibodies, antibody fragments, siRNAs, shRNAs, nucleic acid molecules (RNAs, DNAs, or DNA/RNA hybrids), antisense oligonucleotides, ribozymes, peptides, peptide mimetics, carbohydrates, lipids, microorganisms, natural products, and the like. In some embodiments, an agent can be isolated or, in other embodiments, not isolated. As a non-limiting example, an agent can be a library of agents. If a mixture of agents is found to be a modulator, the pool can then be further purified into separate components to determine which components are in fact modulators of a target activity.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to various salts of amino acids, amino acid derivatives (such as amides), and/or compounds generated by making substitutions on amino acid side chains. Amino acids, including carboxy- and/or amino-terminal amino acids in peptides, can be modified by methylation, amidation, acetylation, and/or substitution with other chemical groups that can, for example, change the peptide's circulating half-life without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids.

Anti-osteopontin therapy: As used herein, the term “anti-osteopontin therapy” refers to any therapy that directly or indirectly inhibits or reduces osteopontin activity by using therapeutic agents identified herein or methods known in the art. Exemplary therapeutic agents suitable for anti-osteopontin therapy include, but are not limited to, chemical compounds, small molecules, proteins or peptides, antibodies, co-crystals, nano-crystals, microorganisms (e.g., virus, bacteria, fungi, etc.), nucleic acids (e.g., DNAs, RNAs, DNA/RNA hybrids, siRNAs, shRNAs, miRNAs, ribozymes, aptamers, etc.), carbohydrates (e.g. mono-, di-, or poly-saccharides), lipids (e.g., phospholipids, triglycerides, steroids, etc.), natural products, any combination thereof. Therapeutic agents can also be designed using computer-based rational drug design methods.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biomarker: As used herein, the term “biomarker” refers to any substance that can be used as an indicator of a biologic state. Typically, a biomarker is a characteristic that is objectively measured and evaluated as an indicator of normal biologic processes, pathogenic processes, or pharmacologic responses to a therapeutic intervention. In some embodiments, a biomarker is a substance or molecule that can be used to indicate the state of tumor growth or metastasis. In some embodiments, a biomarker can be used for diagnosis of tumor. In some embodiments, a biomarker is not necessarily directly involved in activating or recruiting bone marrow cells. Biomarkers can be cells, proteins (e.g., receptors), peptides, polypeptides, nucleic acids (e.g., DNA, RNA), organic or inorganic compounds.

Combination therapy: The term “combination therapy”, as used herein, refers to those situations in which two or more different pharmaceutical agents are administered in overlapping regimens so that the subject is simultaneously exposed to both agents.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Control animal host: As used herein, the term “control animal host” refers to an otherwise similar animal host that has not been selected or modified to bear a variable being tested. In some embodiments, a control animal host is an otherwise similar animal host that has not been selected or modified to bear one or more cells that instigate the growth of an otherwise indolent tumor. In some embodiments, a control animal host is an otherwise similar animal host that has not been selected or modified to bear one or more cells or compositions that secret or release a protein (e.g., OPN) or other factors that promote tumor growth or metastasis.

Dosing regimen: A “dosing regimen”, as that term is used herein, refers to a set of unit doses (at least one and often more than one) that are administered individually separated by periods of time. The set of doses, e.g., a recommended set of doses (i.e., amounts, timing, route of administration, etc.) for a particular therapeutic agent constitutes its dosing regimen.

Indolent tumors: As used herein, the term “indolent tumors” (also referred to as “indolent cells” or “indolent responders”) refers to cells or tumors that maintain a balance between proliferation and apoptosis/necrosis such that there is no overall change in size or mass over time; transformed cells that maintain quiescence for a protracted period of time; cells or tumors that exhibit a long latency before exhibiting a growth phase; cells or tumors with slow growth kinetics; cells or tumors that would otherwise die or be cleared upon transplantation/injection into a host; cells or tumors that do not manifest as growing masses in a living animal (for example, in some transgenic mice, tumors are not apparent until a very late stage (e.g., the mouse is more than 10 months old), the mouse is moribund, or the mouse is euthanized and undergoes vivisection); tumors or cell lines that do not display a desmoplastic reaction (stromal desmoplasia); tumors that are not well vascularized; cells or tumors that are not capable of recruiting significant numbers of blood vessels, fibroblasts or myofibroblasts; cell lines or tumors that are not capable activating the bone marrow; cells or tumors that are not capable of instigating the growth of other cells or tumors; cells or tumors that are not capable of recruiting bone marrow-derived cells into their stroma; cells or tumors that do not display histopathology that is consistent with adenocarcinomas; cells or tumors that do not express osteopontin; cells or tumors that do not form growing metastatic colonies upon dissemination from the primary tumor, or after injection per current experimental models of metastasis. The “indolent” tumors, “indolent” cells or “indolent responders” can be human tumor cell lines (e.g., breast cancer cell lines or prostate cancer cell lines), fresh or frozen human tumor surgical samples, fresh or frozen human biopsy samples, human tumor cells, premalignant and preneoplastic and/or dysplastic cells or tissues, surgical or biopsy samples that grew as xenografts in a host mouse and are passaged again into another host animal, single cell suspensions derived from human biopsy or surgical samples, any genetically modified cell types (not even necessarily tumor), mouse tumor cell lines, mouse tumors that are passaged into another host, spontaneously-arising tumors from transgenic mouse models of tumor initiation and progression.

Instigation: As used herein, the term “instigation” refers to a stimulation process by growing tumors, tumor-associated or tumorigenic cells, proteins or other factors secreted by tumors or tumor cells, or a physical process (e.g., surgical or other types of wounds). Typically, instigation refers to systemic instigation which is a stimulation process involving action-at-a-distance. In some embodiments, systemic instigation is mediated by host systemic environment. In some embodiments, instigation refers to systemic stimulation of growth of a distant, otherwise indolent tumor. In some embodiments, instigation includes activation of bone marrow cells (BMCs). In some embodiments, instigation includes mobilization and incorporation of BMCs or bone marrow-derived cells or circulating blood cells into the stroma of distant, otherwise-indolent tumors.

Instigators: As used herein, the term “instigators” refers to any cells, tumors or processes that enhance, support or induce the growth and/or metastasis of another tumor or cell, in particular, in a systemic fashion. The “instigators” or “instigating” cells or tumors include any cells or tumors that proliferate in an animal host and the proliferation of such cells or tumors enhances, supports or induces the growth and/or metastasis of another tumor or cell, in particular, in a systemic fashion. In particular, the “instigators” or “instigating” cells or tumors can be human tumor cell lines, fresh or frozen human tumor surgical samples, fresh or frozen human biopsy samples, human tumor cells, surgical or biopsy samples that grew as xenografts in a host mouse and are passaged again into another host animal, single cell suspensions derived from human biopsy or surgical samples, any cell type (tumorigenic or non-tumorigenic) that are genetically modified to increase the propensity for tumor formation. In some embodiments, the instigating cells are human tumor cells are such as, for example, BPLER cells, MDA-MB-231 breast cancer cells and MCF7Ras breast cancer cells. Without limitation, the instigating cells or tumors may arise from epithelium, endothelium, or mesothelium. The instigating cells or tumors may be an adenocarcinoma, a squamous cell carcinoma, a sarcoma, a melanoma, a neuroendocrine tumor, a hematopoietic tumor, a lymphoma, a leukemia or a premalignant, preneoplastic and/or dysplastic cell or tissue. Without limitation, the tissue of origin can be lung, liver, breast, prostate, kidney, colon, testis, ovary, stomach, pancreas, thyroid, skin, bone, uterus, or brain.

Introducing: As used herein, the term “introducing,” when used in connection in with introducing a cell or tumor into an animal host, encompasses injecting, implanting, growing, transplanting, or any other methods known in the art.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated, e.g., on a w/w or molar basis. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components (except, in some embodiments, a solvent such as water and/or a counterion). As used herein, the term “isolated cell” refers to a cell not contained in a multi-cellular organism.

Modulator: As used herein, the term “modulator” refers to a compound that alters or elicits an activity. For example, the presence of a modulator may result in an increase or decrease in the magnitude of a certain activity compared to the magnitude of the activity in the absence of the modulator. In certain embodiments, a modulator is an inhibitor, which decreases the magnitude of one or more activities. In certain embodiments, an inhibitor completely prevents one or more biological activities. In certain embodiments, a modulator is an activator, which increases the magnitude of at least one activity. In certain embodiments the presence of a modulator results in an activity that does not occur in the absence of the modulator. In certain embodiments the activity is a biological pathway or process, e.g., tumor growth or metastasis, cell migration, cell differentiation, formation of a desmoplastic stroma, etc. It will be appreciated that such activities may comprise multiple activities that may interact with one another.

Modification state: As used herein, the term “modification state” (also “modification status”), when used in connection with a protein or polypeptide, refers to a post-translational modification status, such as, for example, phosphorylation state or status, glycosylation state or status.

Non-instigator: As used herein, a “non-instigator” refers to any cells, tumors or processes that do not act as instigators as described above. The “non-instigators” or “non-instigating” cells or tumors include any cells or tumors that proliferate in an animal host and the proliferation of such cells or tumors does not enhance, support or induce the growth and/or metastasis of another tumor or cell, in particular, in a systemic fashion. In some embodiments, “non-instigating” cells or tumors can be human tumor cell lines, fresh or frozen human tumor surgical samples, fresh or frozen human biopsy samples, human tumor cells. In some embodiments, non-instigating cells are human tumor cells such as, for example, breast cancer cells (e.g., BT474 (ATCC designation HTB-20™)) or prostate cancer cells (e.g. PC3). Without limitation, non-instigating cells or tumors may arise from epithelium, endothelium, or mesothelium. Non-instigating cells or tumors may be an adenocarcinoma, a squamous cell carcinoma, a sarcoma, a melanoma, a neuroendocrine tumor, a hematopoietic tumor, a lymphoma, a leukemia, or a premalignant, preneoplastic and/or dysplastic cell or tissue. Without limitation, the tissue of origin can be lung, liver, breast, prostate, kidney, colon, testis, ovary, stomach, pancreas, thyroid, skin, bone, uterus, or brain.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention may be specifically directed to “unmodified nucleic acids,” meaning nucleic acids (e.g., polynucleotides and residues, including nucleotides and/or nucleosides) that have not been chemically modified in order to facilitate or achieve delivery.

Osteopontin: As used herein, the term “osteopontin (OPN)” includes osteopontin or osteopontin-like proteins. OPN is a secreted, acidic, calcium-binding, phosphorylated glycoprotein. OPN is also known as secreted phosphoprotein 1, bone sialoprotein 1, and early T-lymphocyte activation 1.

Osteopontin receptors: As used herein, the term “osteopontin receptors” includes any receptors that mediate an osteopontin function. Exemplary osteopontin receptors include, but are not limited to, members of the integrin family, such as, for example, α5β1, αvβ3, αvβ5, α9β1, and αvβ6, and other receptors such as CD44.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, etc. In some embodiments, proteins may comprise natural amino acids, non-natural amino acids, synthetic amino acids, and combinations thereof. The term “peptide” is generally used to refer to a polypeptide having a length of less than about 100 amino acids.

Small molecule: In general, a “small molecule” is understood in the art to be an organic molecule that is less than about 5 kilodaltons (Kd) in size. In some embodiments, the small molecule is less than about 4 Kd, about 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Subject: As used herein, the term “subject” or “patient” refers to any organism to which compositions in accordance with the invention may be administered, e.g., for experimental, diagnostic, prophylactic, and/or therapeutic purposes. Typical subjects include animals (e.g., mammals such as mice, rats, rabbits, non-human primates, and humans; insects; worms; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: As used herein, the term “susceptible to” means having an increased risk for and/or a propensity for (typically based on genetic predisposition, environmental factors, personal history, or combinations thereof) something, i.e., a disease, disorder, or condition, than is observed in the general population. The term takes into account that an individual “susceptible” for a disease, disorder, or condition may never be diagnosed with the disease, disorder, or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Therapeutic agent: As used herein, the phrase “therapeutic agent” refers to any agent that, when administered to a subject, has a therapeutic effect and/or elicits a desired biological and/or pharmacological effect.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION OF THE INVENTION

The present invention provides systems and methods for studying tumor growth or metastasis. The invention is, in part, based on the discovery that certain tumors (e.g., human tumors) or tumor cells instigate the growth of an otherwise-indolent tumor. In particular, we find that human tumors, such as, breast carcinomas instigate the growth of otherwise-indolent tumor cells, micrometastases, and human tumor surgical specimens, in particular, located at distant anatomical sites. The present inventors further discovered that systemic instigation is mediated at least in part by activated bone marrows. In certain embodiments, the systemic instigation is accompanied by decreased numbers of primitive hematopoietic cells in the bone marrow and, in certain embodiments, results in mobilization of bone marrow cells to the stroma of the distant indolent tumors. In certain embodiments, the bone marrow of hosts bearing instigating tumors is functionally activated prior to mobilization; hence, bone marrow cells from these hosts, when co-injected with indolent cells, mimic the systemic effects imparted by instigating tumors. In certain embodiments, we find that secretion of osteopontin by instigating tumors is necessary for activating the bone marrow and facilitating outgrowth of the distant, otherwise-indolent tumors. The present inventors further discovered that the formation of reactive stroma, a condition that is associated with poor prognosis, can be instigated systemically by instigating tumors or tumor cells via activation of bone marrow cells (e.g., Sca1⁺/cKit⁻ cells).

Thus, the present invention provides inventive methods for characterizing tumor growth and/or metastasis that can be used to identify modulators and/or contributors of tumor growth or metastasis, as well as diagnostic and/or therapeutic agents for use in monitoring and/or modulating tumor growth and/or metastasis.

### Indolent Responders

Indolent responders in accordance with the present invention include, but are not limited to, the following: cells or tumors that maintain a balance between proliferation and apoptosis such that there is no overall change in size or mass over time; transformed cells that maintain quiescence for a protracted period of time; cells or tumors that exhibit a long latency before exhibiting a growth phase; cells or tumors with slow growth kinetics; cells or tumors that would otherwise die or be cleared upon transplantation/injection into a host; cells or tumors that do not manifest as growing masses in a living animal (for example, in some transgenic mice, tumors are not apparent until a very late stage (e.g., the mouse is more than 10 months old), the mouse is moribund, or the mouse is euthanized and undergoes vivisection); tumors or cell lines that do not display a desmoplastic reaction (stromal desmoplasia); tumors that are not well vascularized; cells or tumors that are not capable of recruiting significant numbers of blood vessels, fibroblasts or myofibroblasts; cell lines or tumors that are not capable activating the bone marrow; cells or tumors that are not capable of instigating the growth of other cells or tumors; cells or tumors that are not capable of recruiting bone marrow-derived cells into their stroma; cells or tumors that do not display histopathology that is consistent with adenocarcinomas; cells or tumors that do not express osteopontin; cells or tumors that do not form growing metastatic colonies upon dissemination from the primary tumor, or after injection per current experimental models of metastasis. The “indolent” tumors, “indolent” cells or “indolent responders” can be human tumor cell lines, fresh or frozen human tumor surgical samples, fresh or frozen human biopsy samples, human tumor cells, premalignant and preneoplastic and/or dysplastic cells or tissues, surgical or biopsy samples that grew as xenografts in a host mouse and are passaged again into another host animal, single cell suspensions derived from human biopsy or surgical samples, any genetically modified cell types (not even necessarily tumor), mouse tumor cell lines, mouse tumors that are passaged into another host, spontaneously-arising tumors from transgenic mouse models of tumor initiation and progression.

In some embodiments, an indolent tumor is an adenocarcinoma, a sarcoma, a melanoma, a Wilms' tumor, a lymphoma, or a benign tumor. In some embodiments, an indolent tumor is a biopsy or surgical sample derived from a tissue such as, for example, breast, lymph node, prostate, kidney, lung, liver, gastrointestinal tract, colon, testis, stomach, pancreas, thyroid, or brain. In certain embodiments, an indolent responder is derived from a primary tumor. In certain embodiments, an indolent responder is derived from a metastatic nodule. Exemplary indolent tumors are described in further detail in the Examples. For example, as discussed in Example 13, an exemplary indolent tumor responder is a Her2 positive breast cancer cell line (e.g., SKBR3 (ATCC designation HTB-30™)). Thus, the instigating system according to the present invention can be used to characterize aggressive Her2-positive breast cancers and to identify genes and proteins contributing to the outgrowth and metastasis of Her2-positive cancers and provide therapeutic targets for the treatment of such cancers.

The invention encompasses screening tumor cell lines or surgical samples to identify additional indolent tumors capable of responding to an instigator. Such tumor cell lines or tumors can then be used in the inventive in vivo systemic instigation system for any of the purposes contemplated herein.

### Instigators

Instigators in accordance with the present invention include any cells, tumors or processes that enhance, support or induce the growth and/or metastasis of another tumor or cell, in particular, in a systemic fashion. The “instigators” or “instigating” cells or tumors include any cells or tumors capable of proliferating in an animal host and the proliferation of such cells or tumors enhances, supports or induces the growth and/or metastasis of another tumor or cell, in particular, in a systemic fashion. In particular, the “instigators” or “instigating” cells or tumors can be human tumor cell lines, fresh or frozen human tumor surgical samples, fresh or frozen human biopsy samples, human tumor cells, surgical or biopsy samples that grew as xenografts in a host mouse and are passaged again into another host animal, single cell suspensions derived from human biopsy or surgical samples, any cell type (tumorigenic or non-tumorigenic) that are genetically modified to increase the propensity for tumor formation. In some embodiments, the instigating cells are human tumor cells, such as, for example, BPLER cells, MDA-MB-231 breast cancer cells or MCF7Ras breast cancer cells. Without limitation, the instigating cells or tumors may arise from epithelium, endothelium, or mesothelium. The instigating cells or tumors may be an adenocarcinoma, a squamous cell carcinoma, a sarcoma, a melanoma, a neuroendocrine tumor, a hematopoietic tumor, a lymphoma or leukemia. Without limitation, the tissue of origin can be lung, liver, breast, prostate, kidney, colon, testis, ovary, stomach, pancreas, thyroid, skin, bone, uterus, or brain. In certain embodiments, instigating tumors can be at a relatively early stage of their growth, for example, at a time when these instigating tumors represent less than 0.08% of total body mass. Exemplary instigators are described in further detail in the Examples. The invention encompasses screening tumor cell lines or surgical samples to identify additional cell lines or tumors capable of serving as instigators. Such tumor cell lines or tumors can then be used in the inventive in vivo systemic instigation system for any of the purposes contemplated herein.

In certain embodiments, the instigating cells are cells that are genetically modified to overexpress one or more proteins, such as, for example, growth factors, cytokines, receptors, osteopontins, oncogenes, kinases or combinations thereof. In some embodiments, “overexpress” refers to a detectable increase in expression level relative to the expression level that exists prior to such genetic modification. For example, in some embodiments the expression level is increased from an undetectable level (e.g., a level approximately equal to background signal using a suitable detection system) to a detectable level. In some embodiments, the expression level increases by a factor of 2, 5, 10, 25, or more. In certain embodiments, the instigating cells are cells that are genetically modified to reduce expression or function of a gene. For example, the instigating cells can be genetically modified to reduce the expression or function of a tumor suppressor gene or a metastasis suppressor gene. In some embodiments, small interfering RNA (siRNA), short hairpin RNA (shRNA), or microRNAs (miRNA) can be used to reduce the expression of a tumor suppressor or metastasis suppressor gene in the instigating cells. In certain embodiments, miRNA is used. Typically, microRNAs (miRNA) are single-stranded RNA molecules typically of about 21-23 nucleotides in length that regulate gene expression. miRNAs are processed from primary transcripts known as pri-miRNA to shorter stem-loop structures called pre-miRNA and finally to functional miRNA. Mature miRNA molecules are partially complementary to one or more messenger RNA (mRNA) molecules and downregulate gene expression, often by inhibiting translation or triggering mRNA degradation by the RNA interference machinery. miRNAs regulate a diverse array of biological processes including development, cell proliferation, and development. A number of miRNAs have been shown to behave as oncogenes or tumor suppressor genes in various types of tumors (see, e.g., Blenkiron C. and Miska, E., Hum. Mol. Genet., 16 (1): R106-R113, 2007; Cho, W., Molecular Cancer 6:60, 2007, doi:10.1186/1476-4598-6-60, and references therein). In some embodiments of the invention the instigating cells are cells that have been genetically modified to have increased or decreased expression of an miRNA associated with one or more tumor types. For example, the cells may be genetically modified to have increased expression of an oncogenic miRNA or decreased expression of a tumor suppressing miRNA. In some embodiments, the cells are genetically modified to have reduced expression or activity of an miRNA that naturally inhibits expression of a gene that encodes a growth factor, a receptor, osteopontin, an oncogene, or a kinase. Genetic modifications of interest include modifying or deleting at least a portion of the miRNA gene or engineering the cell to express an antisense RNA that inhibits function of the miRNA. In some embodiments, the cells are genetically modified to have increased expression of an miRNA that naturally inhibits expression of a tumor suppressor gene.

The instigating cells also include mouse tumor cell lines, mouse tumors that are passaged into another host, spontaneously-arising tumors from transgenic mouse models of tumor initiation and progression.

In certain embodiments, an instigator is an instigating process. An exemplary instigating process is wounding (e.g., surgical wounding).

The “instigators” and the “indolent responders” can be two distinct types of tumors, cells, or surgical or biopsy samples. The “instigators” and the “indolent responders” can also be the same types of cells, tumors, or surgical or biopsy samples.

The “instigators” and the “indolent responders” can be introduced concurrently or sequentially. The “instigators” and the “indolent responders” can be introduced at a same anatomical site or at distinct anatomical sites of an animal host. The “instigators” and the “indolent responders” can be mixed together before they are introduced into an animal host.

The “instigators” and the “indolent responders” can be introduced into an animal host by any methods known in the art. For example, the “instigators” and the “indolent responders” can be introduced by subcutaneous injection, implantation, mammary fat pat injection or implantation, intravenous injection, bronchioalveolar lavage, intracardiac injection, intramuscular injection, kidney capsule implantation, mesenteric vein injection, intrafemoral injection, retroorbital injection.

### Animal Hosts

As used herein, an “animal host” includes any animal model suitable for cancer research. For example, animal hosts suitable for the invention can be any mammalian hosts, including primates, cats, dogs, cows, horses, rodents such as, mice, hamsters, rabbits, and rats. In certain embodiments, an animal host used for the invention is a mouse. In particular, the mouse or other animal host used in the practice of the present invention can be immunocompromised. A compromised immune system is desirable in some embodiments of the invention to prevent the mouse or other animal host from rejecting the implanted human cells. Immunocompromised animals, e.g., immunocompromised rodents such as rats, hamsters, rabbits, etc., are known in the art. Examples of immunocompromised mice include SCID mice, nude mice, NOD-SCID, Rag^{1-/-}, and Rag2^{-/-}, mice whose thymus gland has been surgically removed or rendered nonfunctional e.g., through a means such as radiation or chemical agents, and mice whose immune system has been suppressed by drugs or genetic manipulations. Immunocompromised animals, e.g., immunocompromised rodents, can also be generated through the afore-mentioned means, and immunocompromised strains are known. Genetically immunocompromised mice are commercially available, and selection of immunocompromised mice or other animal hosts suitable for purposes of the present invention is within ordinary skill in the art. Also of use are young animals whose immune system is immature and animals that have been rendered tolerant to cells to be implanted. In certain embodiments, an animal host suitable for the invention is a transgenic animal.

As used herein, an “activated animal host” includes any animal host as described above that is capable of enhancing the growth or metastasis of an otherwise indolent tumor compared to an un-activated animal host. Typically, an activated animal host bears an instigator as described above.

In certain embodiments, an activated animal host has an increased level of one or more cytokines (e.g., autocrine, paracrine or endocrine) or chemokines. For example, an activated animal host can be a mouse that has an increased level of one or more cytokines (e.g., autocrine, paracrine or endocrine). In particular embodiments, an activated animal host is a mouse bearing one or more implanted slow-release pellets secreting one or more cytokines. In particular embodiments, an activated animal host is a mouse bearing one or more cells over-expressing one or more cytokines In other embodiments, an activated animal host is a transgenic mouse over-expressing one or more cytokines. As used herein, “cytokines” refer to proteins and peptides that are used in organisms as signaling compounds. In particular, cytokines includes proteins and peptides that are used to allow one cell to communicate with another. Typically, a cytokine can be an autocrine (a cytokine that acts on the cell that secretes it), a paracrine (a cytokine whose action is restricted to the immediate vicinity of a cytokine's secretion), or an endocrine (a cytokine that diffuses to distant regions of the body (e.g., carried by blood or plasma) to affect different tissues). Generally, the cytokine family includes smaller water-soluble proteins and glycoproteins (proteins with an added sugar chain) with a mass typically between 8 and 30 kDa. Exemplary suitable cytokines include, but are not limited to, interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF. Without wishing to be bound by any theory, it is contemplated that cytokines can interact with osteopontin synergistically or additively to mediate systemic instigation.

In some embodiments, suitable animal hosts include “humanized” animals, for example, humanized mice. Humanized animals such as humainzed mice can be generated using methods known in the art (See, e.g., Shultz L. D. et al. Journal of Immunology, 2005, 174: 6477-6489; McCune, J. et al. 1991, Annu. Rev. Immunol. 9: 399-429, Lapidot, T. et al. 1992, Science 255: 1137-1141; Shultz, L. D. et al. 1995, J. Immunol. 154: 180-191; Greiner, D. L. et al. 1998, Stem Cells 16: 166-177). It is further contemplated that humanized animals (e.g., humanized mice) particularly useful for the present invention can be generated by transplanting instigator-activated human cells such as, activated bone marrow stem/progenitor cells (e.g., humanized mice can be generated by transplanting activated bone marrow stem/progenitor cells onto immunodeficient mice, e.g., NOD-SCID mice). Instigator-activated cells are described below.

### Instigator-Activated Cells

It is contemplated that instigators (e.g., instigating tumors, cells and processes) may activate certain cell types in animal hosts. As used herein, instigator-activated cells refer to those cells that undergo alteration in response to signals released by an instigator, thereby acquiring the ability to foster the growth of an otherwise-indolent responding tumor. In some embodiments, instigator-activated cells include those cell types that are enriched or reduced/depleted as compared to un-activated control animal host (e.g., similar non-instigator-bearing animal hosts). As used herein, cell types that are enriched or reduced/depleted refer to those cell types whose populations in activated animal hosts are statistically significently increased or reduced, respectively, as compared to appropriate controls. Appropriate controls include population sizes of the corresponding cell types measured in un-activated control animals. Appropriate controls can be pre-determined reference numbers indicative of normal population sizes of corresponding cell types. Instigator-activated cells can be found and/or purified from various tissues, including, but not limited to, bone marrows, spleens, lymph nodes, lymphoid tissues, and circulationg system (e.g., peripheral blood). It is contemplated that instigator-activated cells may be mobilized into the circulation and become incorporated into the responding tumors.

Activated Bone Marrows

Activated bone marrows or activated bone marrow cells include any bone marrows or bone marrow cells that induce, stimulate, or enhance tumor growth or metastasis. Typically, activated bone marrows can be derived from instigator-bearing animal hosts. In some embodiments, activated bone marrows are characterized by certain cell types that are enriched or reduced/depleted as compared to un-activated control bone marrows. As used herein, cell types that are enriched or reduced/depleted refer to those cell types whose populations in activated bone marrows are statistically significently increased or reduced, respectively, as compared to appropriate controls. Appropriate controls include population sizes of the corresponding cell types measured in un-activated control bone marrows (e.g., bone marrows from similar non-instigator-bearing animal hosts). Appropriate controls can be pre-determined reference numbers indicative of normal population sizes of corresponding cell types. Typically, cell types are determined by the presence or absence of one or more cell surface markers. Suitable cell surface markers include, but are not limited to, Lin, Sca1 (Ly-6A/E), cKit (CD117), CD11b, CD45, Gr1, Tie1, Tie2, VEGFR1 (Flt-1), VEGFR2 (Flk-1, KDR), VEGFR3 (Flt-4), CD1, CD3, CD4, CD8, CD9, CD10, CD16, CD19, CD31 (PECAM), CD34, CD24, CD25, CD27, CD38, CD43, CD44, CD56, CD64, CD86, CD105 (endoglin), CD122, CD133, CD161, CD267 (TAC-I, TNFRSF13C), CD268 (BAFF-R, TNFRSF17), CD269 (BCMA, TNFRSF13C), Mac-1, F4/80, Thy1, Thy1.1, IL7Rβ, IL2Rβ, Coll, B220, Ter119 (Ly-76), NK1.1, DX5, Ly49, KIR, Flt-3, G-CSFR, GM-CSFR, α4β1integrin, α5β1integrin, α9β1integrin, α2β1integrin (VLA-2), αV integrins, VLA-4, Notch, CXCR2, CXCR4, CCR1. In some embodiments, activated bone marrows are characterized by a decrease in its primitive hematopoietic stem cells compared to corresponding un-activated bone marrows. In certain embodiments, activated bone marrows contain reduced primitive hematopoietic cells such as Lin⁻/Sca⁺/cKit⁺ hematopoietic stem cells compared to corresponding un-activated bone marrows. In certain embodiments, the activated bone marrow contains activated Sca⁺/cKit⁻ cells. In certain embodiments, activated bone marrows contain increased number of mobilized Sca⁺/cKit⁻ cells compared to corresponding un-activated bone marrows. As used herein, mobilized Sca⁺/cKit⁻ cells are characterized by increased release of the Sca⁺/cKit⁻ cells into the circulation and their subsequent incorporation into distant responding tumor stroma. In some embodiments, activated bone marrows contain enriched Sca1⁺ cells. In some embodiments, activated bone marrows contain enriched CD11b⁺/CD45 cells. In some embodiments, activated bone marrows contain reduced numbers of VEGFR1⁺ cells. In some embodiments, CD34⁺/CD117⁻ cell type is elevated in the circulation of breast cancer patients with stage IV metastatic disease when compared with cancer-free subjects or patients with ductal carcinoma in situ (DCIS). The CD34⁺/CD117⁻ cell type is thought to be the human counterpart of the mouse Sca1⁺/cKit⁻ cells (Sogo, et al., (1997) “Induction of c-kit Molecules on Human CD34+/c-kit<low Cells: Evidence for CD34+/c-kit<low Cells as Primitive Hematopoietic Stem Cells,” Stem Cells 15:420-429). In certain embodiments, activated bone marrows are characterized by an increased osteopontin level compared to corresponding un-activated bone marrows. Without wishing to be bound by any theories, it is contemplated that enriched and/or reduced cell types in activated bone marrows may contribute to or mediate systemic instigation. It is also contemplated that enriched and/or reduced cell types in activated bone marrows can also be used as indicators or biomarkers of activated bone marrows, systemic instigations, tumor growth or metastasis. Without wishing be bound by any particular theories, it is also contemplated that osteopontin may activate the bone marrow indirectly via modulation of the bone marrow cell niche so that the “activated” host is characterized by an altered hematopoietic cell niche and the cell niche modulation results in activation of the bone marrow in a manner that mediates systemic instigation. Exemplary cell niche modulation is described in Calvi et al. (2003) “Osteoblastic cells regulate the haematopoietic stem cell niche,” Nature 425:841-846.

Typically, activated bone marrow cells may represent a small percentage of the total bone marrow population. In some embodiments, these cells represent about two percent of the total bone marrow population and are unique in their tumor-promoting ability. For example, Sca1⁺/cKit⁻ BMCs from instigator-bearing animals instigate tumor growth, while Sca1⁺/cKit⁻ BMCs from control animals do not support tumor growth. As discussed in the Examples section, as a non-limiting example, while ˜10⁶ admixed, unfractionated BMCs from an instigator-bearing host suffice to instigate a responding tumor, a comparable degree of instigation is achieved by as few as 2×10⁴ of these directly admixed Sca1⁺/cKit⁻ cells.

Without wishing to be bound by any theories, it is contemplated that the bone marrows of hosts bearing instigating tumors are functionally activated and can mimic systemic instigation, which reveals that the BMC precursors of the instigated stroma are altered even prior to their mobilization into the circulation.

Based on the discovery that activated bone marrow cells (e.g., a Sca1⁺/cKit⁻ BMC population) are biologically activated in the marrow of instigator-bearing hosts, it is contemplated that the abundance of Sca1⁺/cKit⁻ cells we observed in the responding tumor stroma of such hosts derives directly from these BMCs. Without wishing to be bound by any theories, it is contemplated that activated bone marrows may promote formation of reactive stroma that support tumor growth and progression. Carcinomas appear to depend on their tissue environment to survive (VanScott, E. J. & Reinertson, R. P. (1961), “The Modulating influence of stromal environment on epithelial cells studied in human autotransplants,” J. Invest. Derm. 36:109-31). In particular, many stromal components (e.g., fibroblasts, myofibroblasts, endothelial cells, hematopoietic cells and inflammatory immune cells) support tumor growth and progression (Bissell et al. (2001), “PUTTING TUMOURS IN CONTEXT,” Nature Reviews Cancer, 1:46-54; Mueller et al. (2004), “Friends or foes—bipolar effects of the tumour stroma in cancer,” Nat. Rev. Cancer 4:839-849; Kalluri et al. (2006) “Fibroblasts in cancer,” Nat. Rev. Cancer 6:392-401). For example, stromal fibroblasts and myofibroblasts within the tumor environment facilitate tumor growth (Olumi et al. (1999) “Carcinoma-associated fibroblasts direct tumor progression of initiated human prostatic epithelium,” Cancer Res. 59:5002-5011; Bhowmick et al. (2004) “Stromal fibroblasts in cancer initiation and progression Nature 432, 332-337; Elenbaas et al. (2001) “Heterotypic signaling between epithelial tumor cells and fibroblasts in carcinoma formation,” Exp. Cell Res. 264:169-84; Orimo et al. (2005) “Stromal fibroblasts present in invasive human breast carcinomas promote tumor growth and angiogenesis through elevated SDF-1/CXCL12 secretion,” Cell 121, 335-348; Allinen et al. (2004) “Molecular characterization of the tumor microenvironment in breast cancer,” Cancer Cell 6:17-32.). Indeed, the formation of stromal desmoplasia (i.e., presence of SMA′ myofibroblasts and collagen deposition) has been found to be an important event in carcinoma progression and an important prognostic indicator of metastatic disease (Mina et al. (2001) “PUTTING TUMOURS IN CONTEXT,” Nature Reviews Cancer, 1:46-54; Mueller et al. (2004) “Friends or foes—bipolar effects of the tumour stroma in cancer,” Nat. Rev. Cancer 4:839-849; Kalluri et al. (2006) “Fibroblasts in cancer,” Nat. Rev. Cancer 6:392-401).

Perhaps the most surprising aspect of the instigation process came from the observations that stromal desmoplasia can be induced systemically. Hence, the cellular composition of a tumor is not dictated exclusively by the neoplastic cells that form the heart of the tumor. Instead, systemic endocrinal signals acting at a distance on the bone marrow can exert strong influence on the composition of the tumor-associated stroma and thus its histopathology.

Without wishing to be bound by any theories, activated bone marrow cells, or a subpopulation of them, undergo alteration in response to signals released by an instigating tumor, thereby acquiring the ability to foster the growth of and the acquisition of a desmoplastic stroma by an otherwise-indolent responding tumor. In other words, it is contemplated that tumors recruit stromal precursors that are dependent on active endocrinal signaling from an instigating tumor.

Without wishing to be bound by any theories, it is also contemplated that activated bone marrows may contribute to metastatic colonization marrow-reconstituting, and resistance to the cytotoxic effects of 5-FU (Randall et al. (1997) “Phenotypic and Functional Changes Induced at the Clonal Level in Hematopoietic Stem Cells After 5-Fluorouracil Treatment,” Blood 89:3596-3606; Randall et al. (2007) “Characterization of a Populaiton of Cells in the Bone Marrow that Phenotypically Mimics Hematopoietic Stem Cells: Resting Stem Cells or Mystery Population?” Stem Cells 1998; 16; 38-48; Klarmann et al. (2003) “Identification of in vitro growth conditions for c-Kit-negative hematopoietic stem cells,” Blood 102:3120-28).

As discussed herein, our results demonstrated that the instigating power of the bone marrow rested with a small minority of cells, in some embodiments, that constitute only ˜2% of the entire BMC population. This cell population, when isolated from the bone marrow of instigating tumor-bearing animals, has very different biological activity than the same cell population isolated from control animals. Activated cell populations can be isolated or identified by assays based on detection of the presence or absence of cell-surface antigens (e.g., Lin, Sca1 (Ly-6A/E), cKit (CD117), CD11b, CD45, Gr1, Tie1, Tie2, VEGFR1 (Flt-1), VEGFR2 (Flk-1, KDR), VEGFR3 (Flt-4), CD1, CD3, CD4, CD8, CD9, CD10, CD16, CD19, CD31 (PECAM), CD34, CD24, CD25, CD27, CD38, CD43, CD44, CD56, CD64, CD86, CD105 (endoglin), CD122, CD133, CD161, CD267 (TAC-I, TNFRSF13C), CD268 (BAFF-R, TNFRSF17), CD269 (BCMA, TNFRSF13C), Mac-1, F4/80, Thy1, Thy1.1, IL7Rα, IL2Rβ, ColI, B220, Ter119 (Ly-76), FCγRII/III, NK1.1, DX5, Ly49, KIR, Flt-3, G-CSFR, GM-CSFR, α4β1integrin, α5β1integrin, α9β1integrin, α2β1integrin (VLA-2), αV integrins, VLA-4, Notch, CXCR2, CXCR4, CCR1) using methods well known in the art (e.g., FACS). Exemplary methods are described in the Examples section.

As discussed herein, activated bone marrow cells are typically mobilized into the circulation and become incorporated into the responding tumors. Thus, it is contemplated that activated bone marrow cells can also be purified from the circulation system (e.g., blood, lymphnoid tissues) and other tissues of animal hosts and human cancer patients.

### Osteopontin

Osteopontin (OPN) is a secreted, acidic, calcium-binding, phosphorylated glycoprotein. OPN is also known as secreted phosphoprotein 1, bone sialoprotein 1, and early T-lymphocyte activation 1. Many isoforms, which originate from alternative splicing of the mRNA and or from various post-translational protein modifications, are known that are either free or bound to the extracellular matrix. Though it was originally purified from bone matrix, it is expressed in numerous body fluids and tissues including milk, urine, activated T-cells, macrophages, fibroblasts, smooth muscle cells, kidney tissue and some tumor cells. Its expression is stimulated in response to several cytokines, growth factors or inflammatory mediators. Increased osteopontin concentrations have been associated with sepsis, metastatic cancer, cerebral ischemia, atherosclerotic plaques, granuloma formation in tuberculosis and autoimmune diseases such as multiple sclerosis (Chabas, D., et al., Science 294 (2001) 1731-1735) or RA (Petrow, P. K., et al., Arthr. Rheum. 43 (2000) 1597-1605). In particular, tumor-derived osteopontin is at least in part soluble.

An exemplary cDNA sequence of human osteopontin is represented by the accession number NM_{—}000582 (SEQ ID NO:1) as shown below.

| | |
|---|---|
| 1 | ctccctgtgt tggtggagga tgtctgcagc agcatttaaa ttctgggagg gcttggttgt |
| 61 | cagcagcagc aggaggaggc agagcacagc atcgtcggga ccagactcgt ctcaggccag |
| 121 | ttgcagcctt ctcagccaaa cgccgaccaa ggaaaactca ctaccatgag aattgcagtg |
| 181 | atttgctttt gcctcctagg catcacctgt gccataccag ttaaacaggc tgattctgga |
| 241 | agttctgagg aaaagcagct ttacaacaaa tacccagatg ctgtggccac atggctaaac |
| 301 | cctgacccat ctcagaagca gaatctccta gccccacaga cccttccaag taagtccaac |
| 361 | gaaagccatg accacatgga tgatatggat gatgaagatg atgatgacca tgtggacagc |
| 421 | caggactcca ttgactcgaa cgactctgat gatgtagatg acactgatga ttctcaccag |
| 481 | tctgatgagt ctcaccattc tgatgaatct gatgaactgg tcactgattt tcccacggac |
| 541 | ctgccagcaa ccgaagtttt cactccagtt gtccccacag tagacacata tgatggccga |
| 601 | ggtgatagtg tggtttatgg actgaggtca aaatctaaga agtttcgcag acctgacatc |
| 661 | cagtaccctg atgctacaga cgaggacatc acctcacaca tggaaagcga ggagttgaat |
| 721 | ggtgcataca aggccatccc cgttgcccag gacctgaacg cgccttctga ttgggacagc |
| 781 | cgtgggaagg acagttatga aacgagtcag ctggatgacc agagtgctga aacccacagc |
| 841 | cacaagcagt ccagattata taagcggaaa gccaatgatg agagcaatga gcattccgat |
| 901 | gtgattgata gtcaggaact ttccaaagtc agccgtgaat tccacagcca tgaatttcac |
| 961 | agccatgaag atatgctggt tgtagacccc aaaagtaagg aagaagataa acacctgaaa |
| 1021 | tttcgtattt ctcatgaatt agatagtgca tcttctgagg tcaattaaaa ggagaaaaaa |
| 1081 | tacaatttct cactttgcat ttagtcaaaa gaaaaaatgc tttatagcaa aatgaaagag |
| 1141 | aacatgaaat gcttctttct cagtttattg gttgaatgtg tatctatttg agtctggaaa |
| 1201 | taactaatgt gtttgataat tagtttagtt tgtggcttca tggaaactcc ctgtaaacta |
| 1261 | aaagcttcag ggttatgtct atgttcattc tatagaagaa atgcaaacta tcactgtatt |
| 1321 | ttaatatttg ttattctctc atgaatagaa atttatgtag aagcaaacaa aatactttta |
| 1381 | cccacttaaa aagagaatat aacattttat gtcactataa tcttttgttt tttaagttag |
| 1441 | tgtatatttt gttgtgatta tctttttgtg gtgtgaataa atcttttatc ttgaatgtaa |
| 1501 | taagaatttg gtggtgtcaa ttgcttattt gttttcccac ggttgtccag caattaataa |
| 1561 | aacataacct tttttactgc ctaaaaaaaa aaaaaaaaaa aaaaaaaaaa aaaaaa |

An osteopontin DNA sequence suitable for the present invention also includes a functional fragment of SEQ ID NO:1, or a nucleotide sequence at least 70%, 75%, 80%, 85%, 90%, 95%, 98%, or 99% identical to SEQ ID NO:1, or a functional fragment thereof.

The protein sequence of human osteopontin is shown as SEQ ID NO:2 Below:

(SEQ ID NO: 2)
MRIAVICFCLLGITCAIPVKQADSGSSEEKQLYNKYPDAVATWLNPDPS
QKQNLLAPQTLPSKSNESHDHMDDMDDEDDDDHVDSQDSIDSNDSDDVD
DTDDSHQSDESHHSDESDELVTDFPTDLPATEVFTPVVPTVDTYDGRGD
SVVYGLRSKSKKFRRPDIQYPDATDEDITSHMESEELNGAYKAIPVAQD
LNAPSDWDSRGKDSYETSQLDDQSAETHSHKQSRLYKRKANDESNEHSD
VIDSQELSKVSREFHSHEFHSHEDMLVVDPKSKEEDKHLKFRISHELDS
ASSEVN.

An osteopontin protein sequence suitable for the present invention also includes a functional fragment of SEQ ID NO:2, or an amino acid sequence at least 70%, 75%, 80%, 85%, 90%, 95%, 98%, or 99% identical to SEQ ID NO:2, or a functional fragment thereof. In some embodiments the fragment is an N-terminal fragment. In some embodiments the fragment is a C-terminal fragment. OPN is subject to various extracellular proteolytic modifications in vivo. Various fragments of OPN have been reported to have biological activity. In some embodiments the fragment has the amino acid sequence of an OPN fragment generated by cleavage at a site for a mammalian protease. In some embodiments, the mammalian protease that cleaves OPN is thrombin. In some embodiments, the fragment comprises residues 167-210. A functional fragment of osteopontin may be at least 10%, 20%, 30%, 40%, 50%, 60%, 70%, 80%, 90% or greater percent of the length of OPN. Polymorphic variants of SEQ ID NO: 1 and 2 are known and may be used in accordance with the present invention.

Detailed functional and structural information of osteopontin is provided in PCT pbulication WO2004001014, the disclosures of which is incorporated by reference herein.

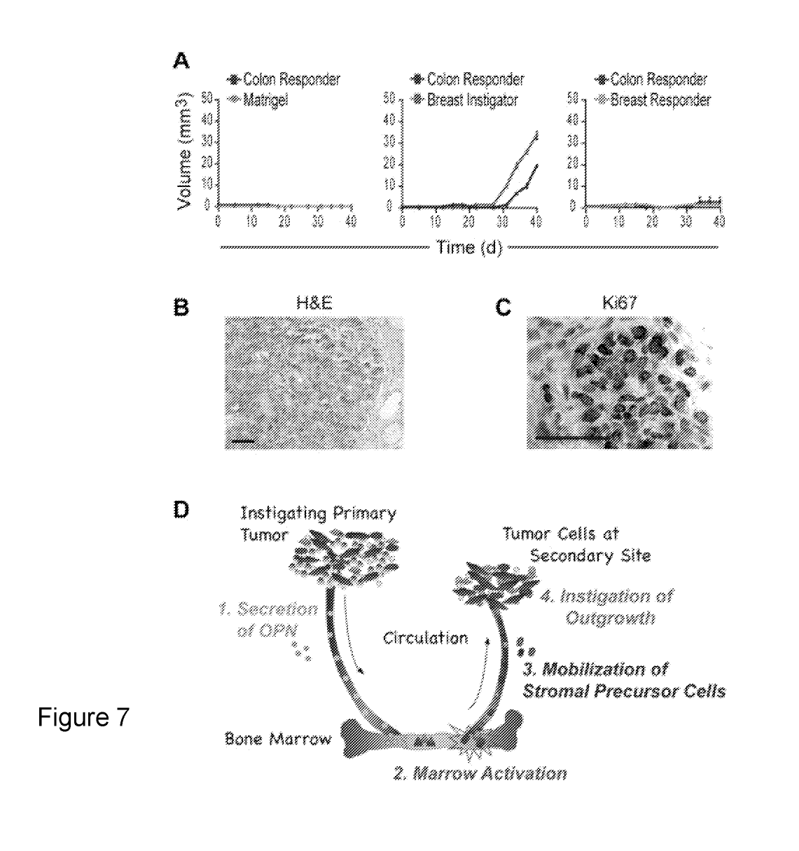

The present inventors discovered that the ability of instigating tumors to perturb the bone marrow environment is, in part, through secretion of OPN, resulting ultimately in the outgrowth of already-established, otherwise indolent tumor cells located at distant sites (FIG. 7D).

OPN expression is significantly elevated in aggressive tumors when compared with counterpart normal tissue or low-grade tumors. Soluble OPN is detected at elevated levels in the blood of many cancer patients with metastatic disease (Chatterjee and Zetter, 2005; Mor et al., 2005; O'Regan and Fleming, 2002; Ramankulov et al., 2007; Rittling and Chambers, 2004; Rudland et al., 2002; Tuck and Chambers, 2001). In the experimental setting, OPN was identified as a critical mediator of breast cancer metastasis. (Adwan et al., 2004; Ariztia et al., 2003; Hayashi et al., 2007; Kang et al., 2003).

We demonstrated that significantly elevated levels of hOPN in the plasma of mice bearing instigating tumors resulted in a subtle yet significant decrease in LSK populations in the bone marrow. Indeed, relatively minor perturbations of bone marrow primitive hematopoietic cells, such as those we observed, have been reported to have a significant physiologic impact (Rossi et al., 2007; Scadden, 2006).

Our results indicate that instigating tumors actively perturb the systemic environment without necessarily affecting their own growth. We identify an additional, novel role for tumor-derived OPN in supporting outgrowth of distantly located, otherwise-weakly tumorigenic cells that is uncoupled from its effects on cell motility within the primary tumor: release of soluble OPN enables instigating tumors to communicate with and perturb the bone marrow, ultimately resulting in the outgrowth of distant, otherwise-indolent tumors, including metastases.

We found that hOPN is important for many aspects of systemic instigation (i.e., perturbation of marrow LSK cells, functional activation of the bone marrow, recruitment of bone marrow-derived cells to the responding tumor and outgrowth of responding tumors or metastases) supports the notion that OPN signaling is an attractive therapeutic target.

### Uses of the Tumor Instigation System

The tumor instigation system of the present invention provides important diagnostic and therapeutic uses. Among other things, the present invention can be used to study aspects of the biology of human tumor specimens that would otherwise be difficult if not impossible to study. For example, the in vivo instigation system of the present invention can be used for accurate classification, diagnosis, staging, prognosis, and determination of treatment of a tumor. In particular, the in vivo instigation system can be used to provide information relating to whether a tumor is more likely to metastasize or be fast-growing. For example, a human tumor can be evaluated using the bilateral instigation protocol, or the protocol in which the tumor sample is implanted beneath the skin, while other weakly-metastatic or indolent cells are injected intravenously into the same host.

In certain embodiments, the present invention provides a method for evaluating the instigating ability of a tumor in an animal host including the steps of: (a) providing an animal host; (b) introducing one or more cells derived from the tumor to be evaluated into the animal host; (c) introducing an otherwise indolent tumor into the animal host; and (d) evaluating the growth of the otherwise indolent tumor to determine the instigating ability of the tumor. In some embodiments, the tumor to be evaluated is a human tumor. In certain embodiments, the tumor to be evaluated is a surgical or biopsy sample taken from a patient. In some embodiments, the method further includes a step of diagnosing the tumor based on the evaluation result from step (d) to determine whether a human tumor is likely to support outgrowth of cells that have disseminated to distant sites from the site of primary tumor growth.

The present invention also provides a method for determining the risk of tumor outgrowth or metastasis for a patient based on the in vivo instigation system. In certain embodiments, the method includes the steps of: (a) providing an animal host; (b) introducing one or more cells derived from the patient bearing the tumor into the animal host; (c) introducing an otherwise indolent tumor into the animal host; and (d) evaluating the growth of the otherwise indolent tumor to determine the risk of tumor outgrowth or metastasis for the patient. In some embodiments, the one or more cells are derived from the tumor. In other embodiments, the one or more cells are derived from the bone marrow of the patient. For example, bone marrow aspirates may be obtained from cancer patients and mixed with indolent cells to determine if their bone marrow is functionally activated and, therefore, likely to support metastasis, e.g., more likely to support metastasis than bone marrow that is not functionally activated or that exhibits a lesser degree of functional activation. In some embodiments, the one or more cells are derived from the blood of a patient. In some embodiments, inventive methods described herein can be used for diagonosis, prognosis or as tests of response to therapy. For example, blood samples can be drawn at the time of diagnosis, surgery, post surgery, during treatment, etc. and the presence of instigating cells or other factors (e.g., instigating cytokines) can be analyzed by methods described herein.

Conversely, the in vivo instigation system of the present invention can be used to evaluate a human surgical or biopsy tumor specimen by testing whether the human tumor is responsive to instigators. One exemplary method includes the steps of: (a) providing an animal host that it is capable of instigating the growth of an otherwise indolent human tumor; (b) introducing a human surgical tumor specimen into the animal host; (c) evaluating the growth of the human surgical tumor specimen in the animal host. In some embodiments, the evaluation of the growth of the human surgical tumor specimen is based on a comparison to the growth of a control tumor on the animal host. The evaluation result from step (c) can be used for diagnostics purposes such as, classification, staging, and determining the risk of metastasis, fast-growing or drug resistance of the human tumor.

In some embodiments, the present invention provides a method for evaluating a human surgical tumor specimen including the steps of: (a) mixing a human surgical tumor specimen with one or more activated bone marrow cells, wherein the one or more activated bone marrow cells are capable of instigating the growth of the human surgical sample on an animal host; (b) introducing the mixture of step (a) into an animal host; and (c) evaluating the growth of the human surgical tumor specimen on the animal host. In some embodiments, the method further includes a step of diagnosing the human tumor based on the evaluation result from step (c). In some embodiments, the diagnosis of the human tumor includes classification, staging, and determining the risk of metastasis, fast-growing or drug resistance of the human tumor. In some embodiments, the one or more activated bone marrow cells suitable for the method are derived from a patient or an animal host bearing a tumor that is capable of enhancing the growth of an otherwise indolent human tumor.

The diagnosis of the human tumor based on the in vivo instigation system can be used to provide prognosis of the tumor (such as drug resistance, etc.). In certain embodiments, the diagnosis of the tumor includes determining a treatment for the tumor. Suitable treatments include, but are not limited to, chemotherapy, radiation therapy, administration of radioactive moieties, surgery, gene therapy, DNA vaccines (or other cancer vaccines) and therapy, antisense-based therapies, RNAi-based therapies including siRNA therapy (e.g., anti-osteopontin therapy as described above), anti-angiogenic therapy, immunotherapy, bone marrow transplants, aptamers and other biologics such as antibodies and antibody variants, receptor decoys and other protein-based therapeutics. In certain embodiments, the present invention also permits the prediction and evaluation of efficacy of therapeutic and treatment regimens. For example, in certain embodiments of the invention, if a tumor is likely to support outgrowth of cells that have disseminated to distant sites from the site of primary tumor growth, more aggressive chemotherapy should be applied. Thus the invention provides a method of selecting a treatment for a human subject suffering from or at risk of a tumor or tumor recurrence, the method comprising steps of: (a) evaluating the subject's tumor in an in vivo systemic instigation system; and (ii) selecting a treatment for the subject based at least in part on the result of step (a). The evaluation may include determining whether the subject's tumor (a) is an indolent tumor that responds to instigation by an increase in tumor growth or metastasis; (b) is capable of instigating one or more otherwise indolent tumors; or (c) supports outgrowth of cells that have disseminated to distant sites from a site of primary tumor growth.

In certain embodiments of the invention, anti-OPN therapy is administered based at least in part on the evaluation of a subject's tumor and/or bone marrow using the in vivo systemic instigation system. For example, in certain embodiments of the invention anti-OPN therapy is administered if, when tested in the in vivo systemic instigation system of the invention (a) the subject's tumor is determined to be an indolent tumor that responds to instigation by an increase in tumor growth or metastasis; (b) the subject's tumor is determined to be capable of instigating one or more otherwise indolent tumors; or (c) the subject's tumor supports outgrowth of cells that have disseminated to distant sites from a site of primary tumor growth. In some embodiments of the invention anti-OPN therapy is administered if the subject's bone marrow is determined to be activated or if activated bone-marrow derived cells are present at elevated levels in its blood.

The present invention also provides a method for evaluating the ability of an agent to inhibit tumor outgrowth or metastasis. For example, a typical method includes the steps of: (a) providing an animal host that it is capable of instigating the growth or metastasis of an otherwise indolent tumor; (b) introducing a tumor into the animal host; (c) administering an agent to the animal host bearing the tumor; and (d) evaluating the ability of the agent to inhibit the growth or metastasis of the tumor. In some embodiments, the method further includes a step of comparing the evaluation result from step (d) to a control to determine whether the agent is capable of inhibiting the tumor outgrowth or metastasis. For example, the ability of the agent to reduce the likelihood or magnitude of tumor outgrowth or metastasis can be evaluated. In some embodiments, the animal host bears a first tumor that is capable of enhancing the growth of the otherwise indolent tumor. In certain embodiments, the agent evaluated by the method of this aspect of the invention is a drug, a compound, a small molecule, an antibody, a cytokine, or a recombinant protein. In certain embodiments multiple agents are evaluated in combination.

In certain embodiments of the invention, the agent used in inventive methods or systems described herein is an anti-tumor agent. Anti-tumor agents, also referred to as chemotherapeutic or anti-cancer agents, include cytotoxic and cytostatic agents. Exemplary anti-tumor agents include, e.g., alkylating agents or agents with an alkylating action, such as cyclophosphamide, chlorambucil, cisplatin, busulfan, melphalan, carmustine, streptozotocin, triethylenemelamine, mitomycin C, etc.; anti-metabolites, such as methotrexate, etoposide, 6-mercaptopurine, 6-thioguanine, cytarabine, 5-fluorouracil (5-FU), capecitabine, dacarbazine, etc.; antibiotics, such as actinomycin D, doxorubicin, anthracycline such as daunorubicin (daunomycin), bleomycin, mithramycin, etc.; vinca alkaloids such as vincristine, vinblastine, etc.; paclitaxel, pactitaxel derivatives, taxanes, glucocorticoids such as dexamethasone or prednisone, nucleoside synthesis inhibitors such as hydroxyurea, amino acid depleting enzymes such as asparaginase, leucovorin and other folic acid derivatives, and similar agents. Additional agents include amifostine, dactinomycin, mechlorethamine (nitrogen mustard), streptozocin, cyclophosphamide, lomustine, gemcitabine, procarbazine, mitomycin, docetaxel, aldesleukin, carboplatin, oxaliplatin, cladribine, camptothecin, irinotecan, 10-hydroxy 7-ethyl-camptothecin, floxuridine, fludarabine, ifosfamide, idarubicin, mesna, interferon beta, interferon alpha, mitoxantrone, topotecan, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil, ethylenimine derivatives, alkyl sulfonates, nitrosoureas, triazenes, anthracyclines, taxanes, pyrimidine analogs, purine analogs, aromatase inhibitors; anti-androgens such as flutamide, nilutamide, bicalutamide, leuprolide, and goserelin, tyrosine or serin/threonine kinase inhibitors (e.g., inhibitors of Abl, c-Kit, insulin receptor family member(s), EGF receptor family member(s), Akt, mTOR (inhibitors specific for mTORC1, mTORC2, or both), Raf kinase family, or phosphatidyl inositol (PI) kinases such as PI3 kinase, PI kinase-like kinase family members, cyclin dependent kinase family members, Aurora kinase family), growth factor receptor antagonists, Hsp90 inhibitors, Ras inhibitors, farnesyltransferase inhibitors, proteasome inhibitors, rapamycin, rapamycin analogs, epipodophyllotoxins, platinum coordination compounds, anti-angiogenic agents, e.g., VEGFR inhibitors such as SU-5416 and SU-6668, VEGF-Trap®, antibodies to VEGF such as bevacizumab (Avastin®), integrin receptor antagonists and integrin antagonists; angiostatin, plasminogen fragments, combretastatin, thalidomide and its anti-angiogenic derivatives such as iMiDs, non-steroidal anti-inflammatory drugs such as aspirin and cyclooxygenase inhibitors, particularly selective inhibitors of cyclooxygenase-2, the fumagillin derivative TNP-470, curcuminoids, and NM-3, a small molecule isocoumarin, endostatin; thrombospondin; plasminogen activator/urokinase inhibitors; urokinase receptor antagonists; heparinases; suramin and suramin analogs; angiostatic steroids; bFGF antagonists; flk-1 and flt-1 antagonists; matrix metalloproteinase inhibitors, pro-apoptotic agents (e.g., apoptosis inducers), etc. In some embodiments at least one of the agents sensitizes tumor cells to activity of a second agent. It will be understood that an agent may fall into more than one of the foregoing categories. It will also be understood that the agent can be a pharmaceutically acceptable salt, pro-drug, or active metabolite of an anti-tumor agent. Furthermore it will be appreciated that agents may be modified, e.g., with targeting moieties, moieties that increase their uptake, biological half-life (e.g., pegylation), etc. Agents may be delivered in or by use of liposomes, polymer-based microspheres or other drug delivery vehicles, etc. In some embodiments of the invention the agent is a cancer vaccine (see, e.g., Schlom, J., et al., Curr Oncol., 14(6):238-45, 2007).

The invention also provides an inhibitor of tumor outgrowth or metastasis determined by evaluation methods as described in various embodiments above.

Furthermore, the present invention also provides a method for evaluating the ability of an agent or stimulus to enhance tumor outgrowth or metastasis. For example, a typical method includes the steps of: (a) providing an animal host that it is capable of instigating the growth or metastasis of an otherwise indolent tumor; (b) introducing a tumor into the animal host; (c) administering an agent to the animal host bearing the tumor; and (d) evaluating the ability of the agent to enhance the growth or metastasis of the tumor. In some embodiments, the method further includes a step of comparing the evaluation result from step (d) to a control to determine whether the agent is capable of enhancing the tumor outgrowth or metastasis. For example, the ability of the agent to increase the likelihood or magnitude of tumor outgrowth or metastasis may be assessed. In some embodiments, the animal host bears a first tumor that is capable of enhancing the growth of the otherwise indolent tumor. In certain embodiments, the agent evaluated by the method of this aspect of the invention is a drug, a compound, a small molecule, an antibody, a cytokine, or a recombinant protein. In certain embodiments multiple agents are evaluated in combination. The method may complement existing methods commonly used in the pharmaceutical or chemical industry to assess the potential of an agent to cause or contribute to cancer. The agent may be a known or suspected carcinogen, a potential therapeutic agent, etc.

The invention further provides a method of evaluating the effect of a stimulus on tumor outgrowth or metastasis comprising the steps of: (a) providing an animal host that it is capable of instigating the growth or metastasis of an otherwise indolent tumor; (b) introducing a tumor into the animal host; (c) subjecting the animal host bearing the tumor to a stimulus; and (d) evaluating the ability of the stimulus to enhance the growth or metastasis of the tumor. In some embodiments, the method further includes a step of comparing the evaluation result from step (d) to a control to determine whether the stimulus is capable of enhancing the tumor outgrowth or metastasis. For example, the ability of the stimulus to increase the likelihood or magnitude of tumor outgrowth or metastasis may be assessed. In some embodiments, the animal host bears a first tumor that is capable of enhancing the growth of the otherwise indolent tumor. In certain embodiments, the stimulus evaluated by the method of this aspect of the invention is wounding, radiation, viral infection, inflammation, etc. In certain embodiments multiple stimuli and/or agents are evaluated in combination. For example, in some embodiments, the animal host is treated with a first agent or first stimulus that capable of enhancing tumor outgrowth or metastasis, and the ability of a second agent or stimulus to inhibit or prevent such outgrowth or metastasis is assessed.

Furthermore, the system may be used for identification and elucidation of genetic or other characteristics of a cell or an animal that contribute to or are associated with increased or decreased likelihood of tumor outgrowth or metastasis, relative to other members of the population. For example, instigators and non-instigators, responders and non-responders can be compared to identify factors or components that differ between them. Such factors or components are candidate modulators of systemic tumor instigation.

In some embodiments, expression profiling studies can be performed on instigators versus non-instigators to identify differentially expressed genes and proteins as candidate modulators of systemic instigation. In some embodiments, instigators and non-instigators suitable for the invention are instigating and non-instigations tumors or tumor cells. In some embodiments, instigating and non-instigations tumors or tumor cells have different origins (e.g., prostate tumor or tumor cells vs. breast tumors or tumor cells). In some embodiments, instigating and non-instigations tumors or tumor cells have the same origin (e.g., both are breast tumors or tumor cells). It is contemplated that instigating and non-instigations tumors or tumor cells of the same origin may reduce the background and false positives in the expression profiling analysis.

In other embodiments, the present invention contemplates methods for characterizing biological or non-biological processes, states, or conditions that may impact instigation process. Without wishing to be bound by any theories, it is contemplated that certain biological or non-biological conditions, states, or processes, such as aging, nutrition, stress, wounding, light cycle, exercise, may impact the instigation process. For example, animal hosts (e.g., mice) that have been exposed to different conditions, are in different physiological states (e.g., at different ages) and that are associated with increased or decreased tumor outgrowth or metastasis can be compared to control hosts. For example, it is contemplated that aging may be associated with decreased capability of instigation. Thus, in some embodiments, aged mice (e.g, older than 8 months, or 1 year) can be compared to young mice (e.g., younger than 8 or 6 months). In some embodiments, mice that have been subjected to caloric restrictions, wounding, stress, exercise, and/or altered light cycles, can be compared to “normal” or control mice (e.g., mice without being subjected to the relevant restrictions or conditions) to determine if any of these conditions affect the instigation process in the animal host (e.g., the ability of instigators to instigate and/or the ability of responders to respond). Having identified conditions that affect instrigation, samples obtained from the affected animal host can be compared to those obtained from normal or control hosts to identify factors (e.g., genes or proteins) that differ in those affacted animals as potential candidate modulators of tumor instigation, outgrowth or metastasis.

Thus, in some embodiments, the present invention also provides methods for identifying modulators of tumor instigation, outgrowth or metastasis. For example, inventive methods may include steps of: (a) providing a sample obtained from an animal host (e.g., mice) that instigate the growth of an otherwise indolent tumor; (b) providing a control sample; and (c) comparing the sample of (a) with the control sample of (b) so as to identify one or more components that differ between the samples, wherein a component that differs between the two samples is identified as a candidate modulator of systemic tumor instigation. In some embodiments, the animal host of step (a) bears an instigator, e.g., instigating cells (experimentally generated tumor cells, cells secreting OPN) or tumors (e.g, implanted tumor samples or spontaneously-arising tumors), and/or surgical wonds, etc. In some embodiments, the control sample is obtained from a control animal host. In some embodiments, the control animal host bears a non-instigator (e.g., non-instigating tumors or cells). In some embodiments, the animal host of step (a) is a young mouse (e.g., younger than 8 or 6 months) and the control animal host is an aged mouse (e.g., older than 8 months or 1 year). In some embodiments, the young and aged mice bear the same tumors or cells.

In some embodiments, inventive methods include steps of: (a) providing a sample obtained from an animal host bearing one or more cells or compositions secreting or releasing OPN; (b) providing a control sample; and (c) comparing the sample of (a) with the control sample of (b) so as to identify one or more components that differ between the samples, wherein a component that differs between the two samples is identified as a candidate modulator of systemic tumor instigation.

The term “sample” encompasses biological material obtained from a subject (e.g., an animal host, a patient), as well as fractions or extracts thereof and/or processed forms derived therefrom. Samples include, but are not limited to, body fluids, cells, tissue lysates, pieces of tissue, nucleic acids or proteins obtained from such fluids, cells, lysates, or pieces of tissue, etc. In some embodiments of the invention the samples comprise blood or a fraction thereof such as plasma or cells. In some embodiments of the invention, the samples comprise cells obtained from the blood, from the bone marrow, and/or from a tumor, etc. In some embodiments, samples comprise BMCs that have been subjected to selection for cells that express of one or more cell surface marker(s) such as those mentioned herein and/or subjected to selection against cells that express one or more cell surface marker(s). In some embodiments, the sample comprises BMC-derived cells present in tumor stroma. In some embodiments, the sample comprises a mixture of two or more samples. For example, a sample can contain cells obtained from the bone marrow admixed with tumor cells. In some embodiments components of a sample are obtained from a single subject. In other embodiments, they are obtained from different subjects and/or cell lines.

One aspect of the invention relates to comparing BMC-derived cells obtained from subjects bearing instigating tumors with BMC-derived cells obtained from control subject. Another aspect of the invention relates to comparing cells circulating in the blood of a subject bearing an instigating tumor with cells circulating in the blood of a control subject. The cells circulating in the blood may be cells that have been mobilized from the bone marrow. In some embodiments the cells are Sca⁺cKit⁻ cells or cells bearing a human cell surface marker functionally equivalent to Sca. Another aspect of the invention relates to comparing blood or plasma obtained from a subject bearing an instigating tumor with blood or plasma obtained from a control subject in order. The afore-mentioned embodiments provide means to identify substances and/or cell types found in elevated or decreased levels in the blood of the former.

The samples can be compared by various methods, including but not limited to, expression profiling methods and other methods described herein and known in the art. Candidate modulators identified in accordance with the present invention can be further tested for their ability to enhance tumor growth and/or metastasis using tumor instigation system described herein or using methods known in the art. Candidate modulators that are determined to be contributors to tumor growth or metastasis may be utilized as targets for further development of anti-tumor therapy. For example, contributors to tumor growth or metastasis can be used to identify therapeutic agents that modulate (e.g., inhibit) the expression of such contributors.

An agent, e.g., a candidate modulator or condition, that enhances, promotes, and/or is required for at least one activity of OPN, e.g, the ability of OPN to mediate systemic instigation, is said to “cooperate with” OPN. In some embodiments, the candidate modulator is tested to determine whether it cooperates with OPN to promote tumor growth or metastasis, e.g., via systemic instigation. For example, the candidate agent may be administered to a subject (e.g., a subject bearing an indolent tumor) in combination with OPN and/or in combination with cells, e.g., non-instigating tumor cells, wherein the cells have optionally been engineered to secrete OPN, and/or in combination with OPN released from a slow-release pellet. The ability of a candidate modulator to mediate systemic instigation in combination with OPN may be assessed. In some embodiments, the ability of the candidate modulator to, e.g., increase tumor growth or metastasis of an otherwise indolent tumor, generate activated bone marrow, mobilize or recruit BMC cells to tumors, etc., is assessed. Optionally, the assessment is performed under varying conditions of OPN expression or level in a subject. In another embodiment, an agent that inhibits the candidate modulator (e.g., an antibody) is administered to a subject bearing an instigating tumor. The ability of the inhibiting agent to inhibit systemic instigation is assessed. In another embodiment, instigating tumor cells are modified to express a shRNA that inhibits expression of a candidate modulator. The effect of such inhibition is assessed. If inhibition reduces instigating ability, the candidate modulator is considered to contribute directly or indirectly to systemic instigation. Agents that inhibit activity of cooperating candidate modulators are candidates for development of anti-tumor therapy. Such therapy may be administered in combination with other therapy, e.g., anti-OPN therapy, described herein.

In some embodiments, contributors to or modulators of tumor growth or metastasis identified in accordance with the present invention may be used to further identify proteins (e.g., receptors, cell surface markers) or cell types to which the modulators or contributors bind. Such receptors, cell surface markers, or cell types identified can be used as targets for development of anti-tumor therapy. For example, cytotoxic therapy could be targeted to such cell types.

In some embodiments, the present invention provides methods of identifying cell types that contribute to systemic instigation including steps of: (a) providing a sample obtained from an animal host bearing one or more cells that instigate the growth of an otherwise indolent tumor; (b) measuring populations of one or more cell types in the sample; (c) identifying at least one cell type whose population is enriched in the sample as compared to a control.

In some embodiments, the present invention provides methods of testing a candidate anti-tumor agent including steps of: (a) administering a candidate anti-tumor agent to an animal host bearing one or more cells that instigate the growth of an otherwise indolent tumor; and (b) determining whether the candidate anti-tumor agent decreases the population of a cell type that enhances outgrowth or metastasis of a tumor or inhibits migration of said cell type to a tumor.

In some embodiments, the present invention provides methods of identifying a candidate biomarker indicative of tumor metastasis or outgrowth including steps of: (a) providing a sample obtained from a subject bearing one or more cells that enhance the growth of a remote tumor; (b) measuring populations of one or more cell types in the sample; (c) identifying at least one cell type whose population is enriched or reduced in the sample as compared to a control, wherein the at least one cell type is a candidate cell marker indicative of tumor metastasis or outgrowth.

In some embodiments, the present invention also provides methods of prognosis of a cancer patient including steps of: (a) providing a sample obtained from a cancer patient; (b) measuring the population of a cell marker indicative of tumor metastasis or outgrowth; and (c) determining the prognosis of the cancer patient based on the result from step (b).

### Expression Profiling

Expression profiling studies can be utilized to compare different samples in accordance with the present invention. For example, expression profiling studies can be performed on activated bone marrow cells to identify differentially expressed genes or proteins that may directly or indirectly contribute to the tumor instigation process. Such differentially expressed genes and/or proteins are candidate modulators of tumor growth and/or metastasis and may be further tested using various methods described herein. In some embodiments, activated bone marrow cells can be isolated from the instigator-bearing hosts. Otherwise similar but un-activated bone marrow cells can be isolated from control non-instigator tumor-bearing hosts (e.g., mice bearing Matrigel control plugs, non-instigating tumors (e.g., PC3 or BT474), or non-instigating derivatives of an instigating cell line or tumor, such as derivatives that express a shRNA that inhibits OPN expression).

Exemplary expression profiling analysis methods and other methods of use are described below. It will be understood that a combination of methods may be used.

### 1. Gene Expression Profiling

Preparation of Pool of Target Nucleic Acids

In order to conduct gene expression profiling analysis, a pool of target nucleic acids are prepared from a sample derived from desirable cells. Any biological sample may be used as a source of target nucleic acids. The pool of target nucleic acids can be total RNA, or any nucleic acid derived therefrom, including each of the single strands of cDNA made by reverse transcription of the mRNA, or RNA transcribed from the double-stranded cDNA intermediate. Methods of isolating target nucleic acids for analysis with an oligonucleotide array or other probes, such as phenol-chloroform extraction, ethanol precipitation, magnetic bead separation, or silica-gel affinity purification, are well known to one of skill in the art.

For example, various methods are available for isolating or enriching RNA. These methods include, but are not limited to, RNeasy kits (provided by Qiagen), MasterPure kits (provided by Epicentre Technologies), charge-switch technology (see, e.g., U.S. Published patent application Nos. 2003/0054395 and 2003/0130499), and TRIZOL (provided by Gibco BRL). The RNA isolation protocols provided by Affymetrix can also be employed in the present invention. See, e.g., GeneChip® EXPRESSION ANALYSIS TECHNICAL MANUAL (701021 rev. 3, Affymetrix, Inc. 2002).

If desired, the pool of target nucleic acids (i.e., mRNA or nucleic acids derived therefrom) can be fractionated to reflect the transcription of gene coding regions. In one example, mRNA is enriched by removing rRNA. Different methods are available for eliminating or reducing the amount of rRNA in a sample. For instance, rRNA can be removed by enzyme digestions. According to the latter method, rRNAs are first amplified using reverse transcriptase and specific primers to produce cDNA. The rRNA is allowed to anneal with the cDNA. The sample is then treated with RNAase H, which specifically digests RNA within an RNA:DNA hybrid.

Target nucleic acids may be amplified before incubation with an oligonucleotide array or other probes. Suitable amplification methods, including, but not limited to, reverse transcription-polymerase chain reaction, ligase chain reaction, self-sustained sequence replication, and in vitro transcription, are well known in the art. Certain methods of the present invention involve detecting the hybridization intensity between target nucleic acids and complementary oligonucleotide probes. To accomplish this, target nucleic acids may be attached directly or indirectly with appropriate and detectable labels. Direct labels are detectable labels that are directly attached to or incorporated into target nucleic acids. Indirect labels are attached to polynucleotides after hybridization, often by attaching to a binding moiety that was attached to the target nucleic acids prior to hybridization. Such direct and indirect labels are well known in the art.

Target nucleic acids may be labeled before, during or after incubation with an oligonucleotide array. Labels may be incorporated during the amplification step by using nucleotides that are already labeled (e.g., biotin-coupled dUTP or dCTP) in the reaction. Alternatively, a label may be added directly to the original nucleic acid sample (e.g., mRNA, cDNA) or to the amplification product after the amplification is completed. Means of attaching labels to nucleic acids are well known to those of skill in the art and include, but are not limited to, nick translation, end-labeling, and ligation of target nucleic acids to a nucleic acid linker to join it to a label. Alternatively, several kits specifically designed for isolating and preparing target nucleic acids for microarray analysis are commercially available, including, but not limited to, the GeneChip® IVT Labeling Kit (Affymetrix, Santa Clara, Calif.) and the Bioarray™ High Yield™ RNA Transcript Labeling Kit with Fluorescein-UTP for Nucleic Acid Arrays (Enzo Life Sciences, Inc., Farmingdale, N.Y.).

Polynucleotides can be fragmented before being labeled with detectable moieties. Exemplary methods for fragmentation include, but are not limited to, heat or ion-mediated hydrolysis.

Oligonucleotide Arrays

Probes suitable for the present invention includes oligonucleotide arrays or other probes that capable of detecting the expression of a plurality of genes (including previously undiscovered genes) by cell of interest.

Oligonucleotide probes used in this invention may be nucleotide polymers or analogs and modified forms thereof such that hybridizing to a pool of target nucleic acids occurs in a sequence specific manner under oligonucleotide array hybridization conditions. As used herein, the term “oligonucleotide array hybridization conditions” refers to the temperature and ionic conditions that are normally used in oligonucleotide array hybridization. In some examples, these conditions include 16-hour hybridization at 45° C., followed by at least three 10-minute washes at room temperature. The hybridization buffer comprises 100 mM MES, 1 M [Na+], 20 mM EDTA, and 0.01% Tween 20. The pH of the hybridization buffer can range between 6.5 and 6.7. The wash buffer is 6×SSPET, which contains 0.9 M NaCl, 60 mM NaH2PO4, 6 mM EDTA, and 0.005% Triton X-100. Under more stringent oligonucleotide array hybridization conditions, the wash buffer can contain 100 mM MES, 0.1 M [Na+], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

As is known by one of skill in the art, oligonucleotide probes can be of any length. Preferably, oligonucleotide probes suitable for the invention are 20 to 70 nucleotides in length. For example, suitable oligonucleotide probes are 25 nucleotides in length. In one embodiment, the nucleic acid probes of the present invention have relatively high sequence complexity. In many examples, the probes do not contain long stretches of the same nucleotide. In addition, the probes may be designed such that they do not have a high proportion of G or C residues at the 3′ ends. In another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. In many embodiments, each probe employed in the present invention does not contain any ambiguous base.

Oligonucleotide probes are made to be specific for (e.g., complementary to (i.e., capable of hybridizing to)) a target sequence. Any part of a target sequence can be used to prepare probes. Multiple probes, e.g., 5, 10, 15, 20, 25, 30, or more, can be prepared for each template sequence. These multiple probes may or may not overlap each other. Overlap among different probes may be desirable in some assays. In many embodiments, the probes for a target sequence have low sequence identities with other template sequences, or the complements thereof. For instance, each probe for a target sequence can have no more than 70%, 60%, 50% or less sequence identity with other target sequences, or the complements thereof. This reduces the risk of undesired cross-hybridization. Sequence identity can be determined using methods known in the art. These methods include, but are not limited to, BLASTN, FASTA, and FASTDB. The Genetics Computer Group (GCG) program, which is a suite of programs including BLASTN and FASTA, can also be used.

Normalization control probes can be added to the array. Normalization control probes can be oligonucleotides exactly complementary to known nucleic acid sequences spiked into the pool of target nucleic acids. Any oligonucleotide sequence may serve as a normalization control probe. For example, the normalization control probes may be created from a template obtained from an organism other than that from which the nucleic acids being analyzed is derived. In some embodiments of the invention, the oligonucleotide array further comprises oligonucleotide probes that are exactly complementary to constitutively expressed genes, or subsequences thereof, that reflect the metabolic state of a cell. Nonlimiting examples of these types of genes are beta-actin, transferrin receptor and glyceraldehyde-3-phosphate dehydrogenase (GAPDH).

In one embodiment of the invention, the pool of target nucleic acids is derived by converting total RNA isolated from the sample into double-stranded cDNA and transcribing the resulting cDNA into complementary RNA (cRNA) using methods described in U.S. Publication No. 20060010513, the teachings of which are incorporated herein in their entirety by reference.

In some embodiments of the invention, the oligonucleotide array further comprises control mismatch oligonucleotide probes for each perfect match probe. The mismatch probes control for hybridization specificity. Preferably, mismatch control probes are identical to their corresponding perfect match probes with the exception of one or more substituted bases. More preferably, the substitution(s) occurs at a central location on the probe. For example, where a perfect match probe is 25 oligonucleotides in length, a corresponding mismatch probe will have the identical length and sequence except for a single-base substitution at position 13 (e.g., substitution of a thymine for an adenine, an adenine for a thymine, a cytosine for a guanine, or a guanine for a cytosine). The presence of one or more mismatch bases in the mismatch oligonucleotide probe disallows target nucleic acids that bind to complementary perfect match probes to bind to corresponding mismatch control probes under appropriate conditions. Therefore, mismatch oligonucleotide probes indicate whether the incubation conditions are optimal, i.e., whether the stringency being utilized provides for target nucleic acids binding to only exactly complementary probes present in the array.

For each target sequence, a set of perfect match probes may be chosen using a variety of strategies. As is known, apparent probes are sometimes not suitable for inclusion in the array. This can be due to the existence of similar subsequences in other regions of the genome, which causes probes directed to these subsequences to cross-hybridize and give false signals. Another reason some apparent probes may not be suitable for inclusion in the array is because they may form secondary structures that prevent efficient hybridization. Finally, hybridization of target nucleic acids with (or to) an array comprising a large number of probes requires that each of the probes hybridizes to its specific target nucleic acid sequence under the same incubation conditions.

An oligonucleotide array may comprise one perfect match probe for a target sequence, or may comprise a probeset (i.e., more than one perfect match probe) for a target sequence. For example, an oligonucleotide array may comprise 1, 5, 10, 25, 50, 100, or more than 100 different perfect match probes for a target sequence. The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the GCG. Any method or software program known in the art may be used to prepare probes for use in the present invention. For example, oligonucleotide probes may be generated by using Array Designer, a software package provided by TeleChem International, Inc (Sunnyvale, Calif.). Another exemplary algorithm for choosing probe sets is described in U.S. Pat. No. 6,040,138, the teachings of which are hereby incorporated by reference. Other suitable means to optimize probesets, which will result in a comparable oligonucleotide array, are well known in the art and may be found in, e.g., Lockhart et al. (1996) Nat. Biotechnol. 14:1675-80 and Mei et al. (2003) Proc. Natl. Acad. Sci. USA 100:11237-42.

The oligonucleotide probes can be synthesized using a variety of methods. Examples of these methods include, but are not limited to, the use of automated or high throughput DNA synthesizers, such as those provided by Millipore, GeneMachines, and BioAutomation. In many embodiments, the synthesized probes are substantially free of impurities. In many other embodiments, the probes are substantially free of other contaminants that may hinder the desired functions of the probes. The probes can be purified or concentrated using numerous methods, such as reverse phase chromatography, ethanol precipitation, gel filtration, electrophoresis, or any combination thereof.

Incubation of Target Nucleic Acids with an Array to form a Hybridization Profile

Incubation reactions can be performed in absolute or differential hybridization formats. In the absolute hybridization format, polynucleotides derived from one sample are hybridized to the probes in an oligonucleotide array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to an oligonucleotide array. The oligonucleotide array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

In the present invention, the incubation conditions should be such that target nucleic acids hybridize only to oligonucleotide probes that have a high degree of complementarity. In some embodiments, this is accomplished by incubating the pool of target nucleic acids with an oligonucleotide array under a low stringency condition to ensure hybridization, and then performing washes at successively higher stringencies until the desired level of hybridization specificity is reached. In other embodiments, target nucleic acids are incubated with an array of the invention under stringent or well-known oligonucleotide array hybridization conditions. In some examples, these oligonucleotide array hybridization conditions include 16-hour hybridization at 45° C., followed by at least three 10-minute washes at room temperature. The hybridization buffer comprises 100 mM MES, 1 M [Na+], 20 mM EDTA, and 0.01% Tween 20. The pH of the hybridization buffer can range between 6.5 and 6.7. The wash buffer is 6×SSPET, which contains 0.9 M NaCl, 60 mM NaH2PO4, 6 mM EDTA, and 0.005% Triton X-100. Under more stringent oligonucleotide array hybridization conditions, the wash buffer can contain 100 mM MES, 0.1 M [Na+], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

Differential Gene Expression Profiling Analysis

Methods used to detect the hybridization profile of target nucleic acids with oligonucleotide probes are well known in the art. In particular, means of detecting and recording fluorescence of each individual target nucleic acid-oligonucleotide probe hybrid have been well established and are well known in the art, described in, e.g., U.S. Pat. No. 5,631,734, U.S. Publication No. 20060010513, incorporated herein in their entirety by reference. For example, a confocal microscope can be controlled by a computer to automatically detect the hybridization profile of the entire array. Additionally, as a further nonlimiting example, the microscope can be equipped with a phototransducer attached to a data acquisition system to automatically record the fluorescence signal produced by each individual hybrid.

It will be appreciated by one of skill in the art that evaluation of the hybridization profile is dependent on the composition of the array, i.e., which oligonucleotide probes were included for analysis. In some embodiments the hybridization profile is evaluated by measuring the absolute signal intensity of each location on the array. Alternatively, the mean, trimmed mean (i.e., the mean signal intensity of all probes after 2-5% of the probesets with the lowest and highest signal intensities are removed), or median signal intensity of the array may be scaled to a preset target value to generate a scaling factor, which will subsequently be applied to each probeset on the array to generate a normalized expression value for each gene (see, e.g., Affymetrix (2000) Expression Analysis Technical Manual, pp. A5-14). Conversely, where the array further comprises control oligonucleotide probes, the resulting hybridization profile may be evaluated by normalizing the absolute signal intensity of each location occupied by a test oligonucleotide probe by means of mathematical manipulations with the absolute signal intensity of each location occupied by a control oligonucleotide probe. Typical normalization strategies are well known in the art, and are included, for example, in U.S. Pat. No. 6,040,138 and Hill et al. (2001) Genome Biol. 2(12):research0055.1-0055.13.

Signals gathered from oligonucleotide arrays can be analyzed using commercially available software, such as those provide by Affymetrix or Agilent Technologies. Controls, such as for scan sensitivity, probe labeling and cDNA or cRNA quantitation, may be included in the hybridization experiments. The array hybridization signals can be scaled or normalized before being subjected to further analysis. For instance, the hybridization signal for each probe can be normalized to take into account variations in hybridization intensities when more than one array is used under similar test conditions. Signals for individual target nucleic acids hybridized with complementary probes can also be normalized using the intensities derived from internal normalization controls contained on each array. In addition, genes with relatively consistent expression levels across the samples can be used to normalize the expression levels of other genes.

To identify genes that confer or correlate with a particular phenotype or characteristic, such as tumor instigating ability, a gene expression profile of a sample derived from test cells (e.g., BMCs or circulating blood cells, or a selected fraction thereof, derived from a subject bearing an instigating tumor), is compared to a control profile derived from control cells isolated from a comparable sample from a control subject, e.g., a subject not bearing an instigating tumor. For example, the method may include the following: 1) providing a sample obtained from a subject bearing an instigating tumor and a sample obtained from a subject not bearing an instigating tumor; 2) isolating, processing, and hybridizing total RNA from the first sample to a first oligonucleotide array; 3) isolating, processing, and hybridizing total RNA from the second sample to a second oligonucleotide array; and 4) comparing the resulting hybridization profiles to identify the sequences that are differentially expressed between the first and second samples.

The subsequently identified genes may be blasted against various databases to determine whether they are known genes or unknown genes. If genes are known, pathway analysis can be conducted based on the existing knowledge in the art. Both known and unknown genes are further confirmed or validated by various methods known in the art. For example, the identified genes may be manipulated (e.g., up-regulated or down-regulated) to determine whether doing so induces or suppresses instigating ability and/or tumor growth or metastasis.

### 2. Differential Protein Expression Profiling Analysis

The present invention also provides methods for identifying differentially expressed proteins by protein expression profiling analysis. Protein expression profiles can be generated by any method permitting the resolution and detection of proteins from cells of interest. Methods with higher resolving power are generally preferred, as increased resolution can permit the analysis of greater numbers of individual proteins, increasing the power and usefulness of the profile. A sample can be pre-treated to remove abundant proteins from a sample, such as by immunodepletion, prior to protein resolution and detection, as the presence of an abundant protein may mask more subtle changes in expression of other proteins, particularly for low-abundance proteins. A sample can also be subjected to one or more procedures to reduce the complexity of the sample. For example, chromatography can be used to fractionate a sample; each fraction would have a reduced complexity, facilitating the analysis of the proteins within the fractions.

Useful methods for simultaneously resolving and detecting several proteins include, but are not limited to, array-based methods; mass-spectrometry based methods; and two-dimensional gel electrophoresis based methods.

Protein arrays generally involve a significant number of different protein capture reagents, such as antibodies or antibody variable regions, each immobilized at a different location on a solid support. Such arrays are available, for example, from Sigma-Aldrich as part of their Panorama™ line of arrays or from RayBiotech, Inc. The array is exposed to a protein sample and the capture reagents selectively capture the specific protein targets. The captured proteins are detected by detection of a label. For example, the proteins can be labeled before exposure to the array; detection of a label at a particular location on the array indicates the detection of the corresponding protein. If the array is not saturated, the amount of label detected may correlate with the concentration or amount of the protein in the sample. Captured proteins can also be detected by subsequent exposure to a second capture reagent, which can itself be labeled or otherwise detected, as in a sandwich immunoassay format. In some embodiments, the array is a cytokine detection array.

Mass spectrometry-based methods include, for example, matrix-assisted laser desorption/ionization (MALDI), Liquid Chromatography/Mass Spectrometry/Mass Spectrometry (LC-MS/MS) and surface enhanced laser desorption/ionization (SELDI) techniques. For example, a protein profile can be generated using electrospray ionization and MALDI. SELDI, as described, for example, in U.S. Pat. No. 6,225,047, incorporates a retention surface on a mass spectrometry chip. A subset of proteins in a protein sample are retained on the surface, reducing the complexity of the mixture. Subsequent time-of-flight mass spectrometry generates a “fingerprint” of the retained proteins.

In methods involving two-dimensional gel electrophoresis, proteins in a sample are generally separated in a first dimension by isoelectric point and in a second dimension by molecular weight during SDS-PAGE. By virtue of the two dimensions of resolution, hundreds or thousands of proteins can be simultaneously resolved and analyzed. The proteins are detected by application of a stain, such as a silver stain, or by the presence of a label on the proteins, such as a Cy2, Cy3, or Cy5 dye. To identify a protein, a gel spot can be cut out and in-gel tryptic digestion performed. The tryptic digest can be analyzed by mass spectrometry, such as MALDI. The resulting mass spectrum of peptides, the peptide mass fingerprint or PMF, is searched against a sequence database. The PMF is compared to the masses of all theoretical tryptic peptides generated in silico by the search program. Programs such as Prospector, Sequest, and MasCot (Matrix Science, Ltd., London, UK) can be used for the database searching. For example, MasCot produces a statistically-based Mowse score indicates if any matches are significant or not. MS/MS can be used to increase the likelihood of getting a database match. CID-MS/MS (collision induced dissociation of tandem MS) of peptides can be used to give a spectrum of fragment ions that contain information about the amino acid sequence. Adding this information to a peptide mass fingerprint allows Mascot to increase the statistical significance of a match. It is also possible in some cases to identify a protein by submitting only a raw MS/MS spectrum of a single peptide.

A recent improvement in comparisons of protein expression profiles involves the use of a mixture of two or more protein samples, each labeled with a different, spectrally-resolvable, charge- and mass-matched dye, such as Cy3 and Cy5. This improvement, called fluorescent 2-dimensional differential in-gel electrophoresis (DIGE), has the advantage that the test and control protein samples are run in the same gel, facilitating the matching of proteins between the two samples and avoiding complications involving non-identical electrophoresis conditions in different gels. The gels are imaged separately and the resulting images can be overlaid directly without further modification. A third spectrally-resolvable dye, such as Cy2, can be used to label a pool of protein samples to serve as an internal control among different gels run in an experiment. Thus, all detectable proteins are included as an internal standard, facilitating comparisons across different gels.

### Test Agents

Various candidate agents can be tested in accordance with the present invention. Exemplary test agents include, but are not limited to, chemical compounds, small molecules, proteins or peptides, antibodies, co-crystals, nano-crystals, microorganisms (e.g., virus, bacteria, fungi, etc.), nucleic acids (e.g., DNAs, RNAs, DNA/RNA hybrids, siRNAs, shRNAs, miRNAs, ribozymes, aptamers, etc.), carbohydrates (e.g. mono-, di-, or poly-saccharides), lipids (e.g., phospholipids, triglycerides, steroids, etc.), natural products, any combination thereof. Candidate agents can also be designed using computer-based rational drug design methods. Typically, a plurality of test agents (e.g., libraries of candidate agents) are tested in screening assays for potential modulators. In some embodiments, test agents are biodegradable and/or biocompatible.

Peptides

In some embodiments, random peptide libraries can be screened in accordance with the invention. Exemplary peptide libraries include but not limited to those produced by recombinant bacteriophage, for example, Scott and Smith, Science, 249:386-390 (1990); Cwirla et al., Proc. Natl. Acad. Sci., 87:6378-6382 (1990); Devlin et al., Science, 249:404-406 (1990), or a chemical library. Using the “phage method” very large libraries can be constructed (10⁶-10⁸ chemical entities). A second approach uses primarily chemical methods, of which the Geysen method (Geysen et al., Molecular Immunology 23:709-715 (1986); Geysen et al. J. Immunologic Method 102:259-274 (1987)) and the method of Fodor et al. (Science 251:767-773 (1991)) are examples. Furka et al. 14th International Congress of Biochemistry, Volume 5, Abstract FR:013 (1988); Furka, Int. J. Peptide Protein Res. 37:487-493 (1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as modulators.

In some embodiments, synthetic libraries (Needels et al., Proc. Natl. Acad. Sci. USA 90:10700-4 (1993); Ohlmeyer et al., Proc. Natl. Acad. Sci. USA 90:10922-10926 (1993); Lam et al., International Patent Publication No. WO 92/00252; Kocis et al., International Patent Publication No. WO 9428028, each of which is incorporated herein by reference in its entirety), and the like can be used in accordance with the present invention. Once a potential modulator is identified, chemical analogues can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively synthesized de novo.

Antisense RNAs and Ribozymes

In some embodiments, antisense molecules can be screened in accordance with the invention. Antisense molecules are RNA or single-stranded DNA molecules with nucleotide sequences complementary to a specified mRNA. When a laboratory-prepared antisense molecule is injected into cells containing the normal mRNA transcribed by a gene under study, the antisense molecule can base-pair with the mRNA, preventing translation of the mRNA into protein. The resulting double-stranded RNA or RNA/DNA is digested by enzymes that specifically attach to such molecules. Therefore, a depletion of the mRNA occurs, blocking the translation of the gene product so that antisense molecules find uses in medicine to block the production of deleterious proteins. Methods of producing and utilizing antisense RNA are well known to those of ordinary skill in the art (see, for example, C. Lichtenstein and W. Nellen (Editors), Antisense Technology: A Practical Approach, Oxford University Press (December, 1997); S. Agrawal and S. T. Crooke, Antisense Research and Application (Handbook of Experimental Pharmacology, Volume 131), Springer Verlag (April, 1998); I. Gibson, Antisense and Ribozyme Methodology: Laboratory Companion, Chapman & Hall (June, 1997); J. N. M. Mol and A. R. Van Der Krol, Antisense Nucleic Acids and Proteins, Marcel Dekker; B. Weiss, Antisense Oligonodeoxynucleotides and Antisense RNA Novel Pharmacological and Therapeutic Agents, CRC Press (June, 1997); Stanley et al., Antisense Research and Applications, CRC Press (June, 1993); C. A. Stein and A. M. Krieg, Applied Antisense Oligonucleotide Technology (April, 1998)).

In some embodiments, antisense molecules and ribozymes can be designed based on sequence informations of proteins and genes involved in OPN signaling pathway. For example, antisense molecules and ribozymes can be designed to target OPN and/or OPN receptors as described herein.

The antisense molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding UGGT. Such DNA sequences maybe incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′O-methyl rather than phosphodiesterase linkages within the backbone of the molecule. This concept can be extended by the inclusion of nontraditional bases such as inosine, queosine, and wybutosine, as well as acetyl-, methyl-, thio-, similarly modified forms of adenine, cytidine, guanine, thymine, and uridine which are not as easily recognized by endogenous endonucleases.

Interfering RNAs

RNA interference (RNAi) is a mechanism of post-transcriptional gene silencing mediated by double-stranded RNA (dsRNA), which is distinct from the antisense and ribozyme-based approaches described above. dsRNA molecules are believed to direct sequence-specific degradation of mRNA in cells of various lineages after first undergoing processing by an RNase III-like enzyme called DICER (Bernstein et al., Nature 409:363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length typically with 1-2 nucleotide 3′ overhangs on each strand, resulting in a total length typically of between approximately 21 and 23 nucleotides.

It will also be appreciated that siRNAs can have a range of lengths, e.g., the double-stranded portion can range from 15-29 nucleotides. It will also be appreciated that the siRNA can have a blunt end or a 3′ overhang at either or both ends. If present, such 3′ overhang is often from 1-5 nucleotides in length.

siRNA has been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, T., Nat. Biotechnol., 20:446-448, May 2002. See also Yu, J., et al., Proc. Natl. Acad. Sci., 99(9), 6047-6052 (2002); Sui, G., et al., Proc. Nail. Acad. Sci., 99(8), 5515-5520 (2002); Paddison, P., et al., Genes and Dev., 16, 948-958 (2002); Brummelkamp, T. et al., Science, 296, 550-553 (2002); Miyagashi, M. and Taira, K., Nat. Biotech., 20, 497-500 (2002); Paul, C., et al., Nat. Biotech., 20, 505-508 (2002).

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. Several other approaches for delivery of siRNA into animals have also proved to be successful. See e.g., McCaffery et al., Nature, 418:38-39 (2002); Lewis et al., Nature_{—}Genetics, 32:107-108 (2002); and Xia et al., Nature Biotech., 20:1006-1010 (2002).

siRNA may consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is desirable to target exons rather than introns, and it may also be particularly desirable to select sequences complementary to regions within the 3′ portion of the target transcript. Generally it is preferred to select sequences that contain approximately equimolar ratio of the different nucleotides and to avoid stretches in which a single residue is repeated multiple times.

siRNA may thus comprise RNA molecules typically having a double-stranded region approximately 19 nucleotides in length typically with 1-2 nucleotide 3′ overhangs on each strand, resulting in a total length of between approximately 21 and 23 nucleotides. As used herein, siRNA also includes various RNA structures that may be processed in vivo to generate such molecules. Such structures include RNA strands containing two complementary elements that hybridize to one another to form a stem, a loop, and optionally an overhang, preferably a 3′ overhang. Typically, the stem is approximately 19 by long, the loop is about 1-20, preferably about 4-10, and more preferably about 6-8 nucleotides long and/or the overhang is typically about 1-20, and preferably about 2-15 nucleotides long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may, but need not, comprise a plurality of U residues, e.g., between 1 and 5 U residues.

In some embodiments, interfering RNA molecules can be designed based on sequence informations of proteins and genes involved in OPN signaling pathway. For example, interfering RNA molecules can be designed to target OPN and/or OPN receptors as described herein.

Suitable siRNAs can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845-7854 (1987) and Scaringe et al., Nucleic Acids Res., 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

Inventive siRNAs may be comprised entirely of natural RNA nucleotides, or may instead include one or more nucleotide analogs and/or modifications as mentioned above for antisense molecules. The siRNA structure may be stabilized, for example by including nucleotide analogs at one or more free strand ends in order to reduce digestion, e.g., by exonucleases. This may also be accomplished by the inclusion. Alternatively, siRNA molecules may be generated by in vitro transcription of DNA sequences encoding the relevant molecule. Such DNA sequences may be incorporated into a wide variety of vectors with suitable RNA polymerase promoters such as T7, T3, or SP6.

Antibodies

In some embodiments, antibodies can be screened in accordance with the present invention. For example, antibodies can be designed to target OPN and/or OPN receptors as described herein.

Antibodies can be generated using methods well known in the art. For example, protocols for antibody production are described by Harlow and Lane, Antibodies: A Laboratory Manual, (1988). Typically, antibodies can be generated in mouse, rat, guinea pig, hamster, camel, llama, shark, or other appropriate host. Alternatively, antibodies may be made in chickens, producing IgY molecules (Schade et al., (1996) ALTEX 13(5):80-85). In some embodiments, antibodies suitable for the present invention are subhuman primate antibodies. For example, general techniques for raising therapeutically useful antibodies in baboons may be found, for example, in Goldenberg et al., international patent publication No. WO 91/11465 (1991), and in Losman et al., Int. J. Cancer 46: 310 (1990). In some embodiments, monoclonal antibodies may be prepared using hybridoma methods (Milstein and Cuello, (1983) Nature 305(5934):537-40.). In some embodiments, monoclonal antibodies may also be made by recombinant methods (U.S. Pat. No. 4,166,452, 1979).

In some embodiments, antibodies suitable for the invention may include humanized or human antibodies. Humanized forms of non-human antibodies are chimeric Igs, Ig chains or fragments (such as Fv, Fab, Fab′, F(ab′)2 or other antigen-binding subsequences of Abs) that contain minimal sequence derived from non-human Ig. Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Riechmann et al., Nature 332(6162):323-7, 1988; Verhoeyen et al., Science. 239(4847):1534-6, 1988.). Such “humanized” antibodies are chimeric Abs (U.S. Pat. No. 4,816,567, 1989), wherein substantially less than an intact human variable domain has been substituted by the corresponding sequence from a non-human species. In some embodiments, humanized antibodies are typically human antibodies in which some CDR residues and possibly some FR residues are substituted by residues from analogous sites in rodent Abs. Humanized antibodies include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human Ig. Humanized antibodies may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (Fc), typically that of a human Ig (Riechmann et al., Nature 332(6162):323-7, 1988; Verhoeyen et al., Science. 239(4847):1534-6, 1988.).

Human antibodies can also be produced using various techniques, including phage display libraries (Hoogenboom et al., Mol Immunol. (1991) 28(9):1027-37; Marks et al., J Mol Biol. (1991) 222(3):581-97) and the preparation of human monoclonal antibodies (Reisfeld and Sell, 1985, Cancer Surv. 4(1):271-90). Similarly, introducing human Ig genes into transgenic animals in which the endogenous Ig genes have been partially or completely inactivated can be exploited to synthesize human antibodies. Upon challenge, human antibody production is observed, which closely resembles that seen in humans in all respects, including gene rearrangement, assembly, and antibody repertoire (Fishwild et al., High-avidity human IgG kappa monoclonal antibodies from a novel strain of minilocus transgenic mice, Nat. Biotechnol. 1996 July; 14(7):845-51; Lonberg et al., Antigen-specific human antibodies from mice comprising four distinct genetic modifications, Nature 1994 April 28; 368(6474):856-9; Lonberg and Huszar, Human antibodies from transgenic mice, Int. Rev. Immunol. 1995; 13(1):65-93; Marks et al., By-passing immunization: building high affinity human antibodies by chain shuffling. Biotechnology (N Y). 1992 July; 10(7):779-83).

Small Molecules and Others

In some embodiments, small molecule libraries (including those designed using rational drug design methods), analogues thereof, are screened in accordance with the present invention. In some embodiments, chemical compounds, drugs, or other natural products can be screened in accordance with the present invention.

### Therapeutic Applications

In some embodiments, the invention provides methods treating a subject in need of treatment using therapeutic agents identified in accordance with the invention. In some embodiments, a subject in need of treatment may be susceptible to, suffering from, and/or exhibiting symptoms of one or more diseases, disorders, and/or conditions associated with cancer, tumor growth or metastasis, or OPN dysfunction. In some embodiments, the invention provides methods of treating (e.g., alleviating, ameliorating, relieving, inhibiting, preventing, delaying onset of, reduce severity of, and/or reducing incidence of) one or more symptoms or features of a disease, disorder, and/or condition associated with tumor growth or metastasis. In general, such methods comprise steps of administering one or more therapeutic agents in accordance with the invention to a subject in need of treatment.

In some embodiments, the present invention provides anti-osteopontin therapy that directly or indirectly inhibits or reduces osteopontin activity using therapeutic agents identified herein or using various methods known in the art, including but not limited to antibody therapy, siRNA therapy, antisense therapy, ribozyme therapy, or therapies using small molecules that inhibit OPN.

In certain embodiments, therapeutic agents in accordance with the invention may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the desired dosage may be delivered by a dosing regimen, in which unit doses are administered individually separated by periods of time.

Therapeutic agents and/or pharmaceutical compositions thereof may be administered either alone or in combination with one or more other anti-tumor agents. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In general, each agent will be administered at a dose and/or on a time schedule determined for that agent. In some embodiments, the invention encompasses the delivery of pharmaceutical compositions in combination with agents that may improve their bioavailability, reduce and/or modify their metabolism, inhibit their excretion, and/or modify their distribution within the body.

In will further be appreciated that therapeutically active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating breast cancer in accordance with the invention may be administered concurrently with another anticancer agent), or they may achieve different effects (e.g., control of any adverse effects).

### Pharmaceutical Compositions

The present invention further provides pharmaceutical compositions comprising one or more therapeutic agents, together with one or more pharmaceutically acceptable excipients. Such pharmaceutical compositions may optionally comprise one or more additional therapeutically-active substances.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

Pharmaceutical compositions may be provided in a pharmaceutical pack together with printed matter related to the composition. The pharmaceutical pack can be any suitable container, e.g., a plastic or cardboard box containing a vial, bottle, or the like having the composition inside and sufficient room to include printed matter. The printed matter related to the composition can, e.g., provide instructions for using the composition for treatment of a suitable subject and/or may indicate that the pharmaceutical composition is indicated for and/or approved for use in a suitable subject. The suitable subject may be, e.g., (i) a subject suffering from an indolent tumor; (ii) a subject suffering from or at risk of a tumor capable of instigating growth or metastasis of an otherwise indolent tumor; (iii) a subject having an activated bone marrow; (iv) a subject exhibiting elevated blood levels of one or more cell types or substances indicative of activated bone marrow; and/or (v) a subject having elevated blood levels of one or more molecules that cooperate with osteopontin to activate bone marrow and/or to systemically instigate tumor growth or metastasis, etc. In some embodiments, the pharmaceutical composition comprises an anti-OPN therapy.

Also provided by the present invention are methods of providing a pharmaceutical composition to a pharmacy or health care provider for use in treating a a subject suffering from or at risk of a tumor, the method comprising steps of (i) providing a pharmaceutical composition to the pharmacy or health care provider; and (ii) providing to the pharmacy or health care provider printed matter related to the pharmaceutical composition, wherein the printed matter contains instructions for use of the pharmaceutical composition for treating (i) a subject suffering from an indolent tumor; (ii) a subject suffering from or at risk of a tumor capable of instigating growth or metastasis of an otherwise indolent tumor; (iii) a subject having an activated bone marrow; (iv) a subject exhibiting elevated blood levels of one or more cell types or substances indicative of an activated bone marrow; and/or (v) a subject having elevated blood levels of one or more molecules that cooperate with OPN to activate bone marrow and/or wherein the printed matter indicates that the pharmaceutical composition is indicated and/or approved for use in (i) a subject suffering from an indolent tumor; (ii) a subject suffering from or at risk of a tumor capable of instigating growth or metastasis of an otherwise indolent tumor; (iii) a subject having an activated bone marrow; (iv) a subject exhibiting elevated blood levels of one or more cell types or substances indicative of activated bone marrow; and/or (v) a subject having elevated blood levels of one or more molecules that cooperate with OPN to activate bone marrow and/or to systemically instigate tumor growth or metastasis. In some embodiments the pharmaceutical composition comprises an anti-OPN agent

“Printed matter” encompasses any type of material intended to be understood by a recipient by reading or visual observation. The material may contain text and/or graphical information. The printed matter may be provided by way of paper, electronically, etc. It may be provided together with the pharmaceutical composition or remotely, e.g., via downloading, accessing a website, or otherwise electronically accessing.

The invention is illustrated by the following non-limiting examples.

### EXAMPLES

### Example 1

### Primary Human Tumors Facilitate Growth of Distant Indolent Tumors

Cell Lines

Generation of HMLER hygro-H-rasV12 and BPLER human mammary epithelial tumor cells has been described (Elenbaas et al., 2001; Hahn et al., 1999; Ince et al., 2007). EpH4Ras murine mammary carcinoma cells were a gift from Ernst Reichmann (Oft et al., 1996). Human mammary carcinoma MDA-MB-231, human prostate carcinoma PC3, and murine Lewis lung carcinoma LLC were obtained from the ATCC (Manassas, Va.) and cultured under standard conditions.

Animals and Tumor Xenografts

Female Nude mice were purchased from Taconic (Hudson, N.Y.); a colony of NOD-SCID mice was maintained in-house. All experiments were performed in accordance with guidelines and regulations of MIT Committee on Animal Care protocol (1005-076-08). Tumor cells were injected s.c. into non-irradiated mice and tumor diameter was measured on the flanks of live Nude mice using calipers; volume was calculated as 4/3pir³.

Immunohistochemistry, Immunofluorescence and Antibodies

Dissected tissues were fixed in 4% (wt/vol) paraformaldehyde 16-18 h, embedded in paraffin and sectioned onto ProbeOn Plus microscope slides (Fisher Scientific, Pittsburgh, Pa.) for immunohistochemistry as described (Sendobry et al., 1997) using Vectastain Elite ABC kits (Vector Laboratories, Burlingame, Calif.). Briefly, tissues were dewaxed, washed in 100% EtOH, treated with Redusol (Biomeda Corp., Foster City, Calif.) 2 min 40° C., incubated with fresh H₂O₂/MeOH (1% vol/vol) 2 min 40° C., blocked with appropriate serum 5 min 40° C., incubated with appropriate antibodies at 4° C. o/n or 40° C. for 20 min, washed, incubated with secondary antibodies 15 min 40° C. For IHC, slides were treated by washing with peroxidase enhancer and incubating with AEC chromogen (Biomeda) 2×10 min, 40° C. All washes were performed with Automation Buffer (Biomeda). Cell nuclei were counterstained with aqueous hematoxylin QS (Vector Laboratories, Burlingame, Calif.) or DAPI (2 μg/ml; Molecular Probes). Slides were mounted with Faramount (Dako Corp.) or ProLong Antifade (Molecular Probes). Antibodies used: anti-GFP (1:100; Abeam), anti-Ki67 (1:50; BD Biosciences), anti-Sca1 (1:50; BioLegends), FITC-anti-goat IgG (1:100; Abeam).

Statistical Analysis

Data are expressed as mean±s.e.m. Data were analyzed by Student's t-test and were considered statistically significant if P<0.05.

Results

We constructed an in vivo experimental model utilizing two human tumor cell lines in order to study systemic mechanisms that might directly facilitate tumor progression. The first, termed here an “instigator”, is the experimentally transformed human mammary epithelial BPLER cell line (Ince et al., 2007), which yields vigorously growing tumor xenografts that closely resemble, in their histopathology, the invasive ductal adenocarcinomas commonly encountered in human breast cancer patients. These xenografts contain abundant stroma, indicating that they are capable of recruiting murine stromal cells. The second, termed here a “responder”, is the experimentally transformed human mammary epithelial cell line, HMLER hygro-H-rasV12 (HMLER-HR; Elenbaas et al., 2001). Only ˜25% of the mice injected with these indolently growing cells form observable tumors when examined 9 weeks after implantation.

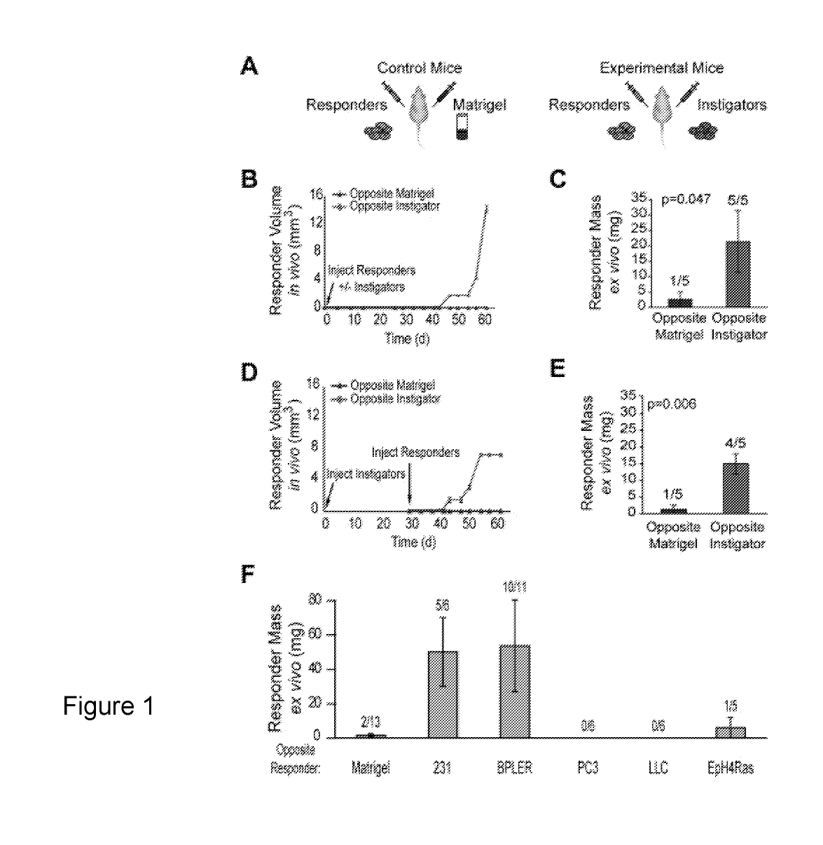

In an initial experiment, instigating BPLER tumor cells were injected subcutaneously into the right flanks of Nude mice, while the responding HMLER-HR cells were injected into the contralateral flanks of these mice (FIG. 1A). In a control group of mice, Matrigel vehicle was injected contralaterally to the indolent responder cells.

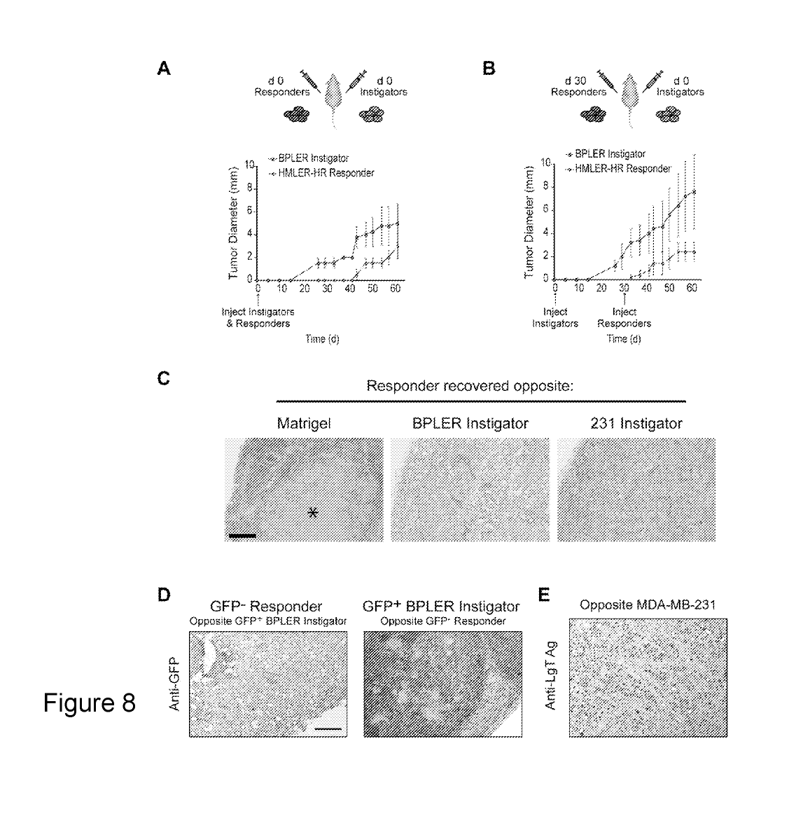

Responder cells injected contralaterally to Matrigel plugs did not form externally measurable tumors during the 9 week experimental time period that followed (FIG. 1B); however, after surgical exposure of the subcutaneous space at the experimental end point, we could recover small masses in 20% of the injected mice (FIG. 1C). Strikingly, however, when the responder cells were injected opposite instigating tumors, the responder cells formed actively growing tumors after a lag phase of ˜40 days (FIG. 1B), at which time the instigating tumors were only ˜3.7 mm in diameter (FIG. 8A). The average final mass of these responding tumors was ˜ten-fold that of the responding cell plugs recovered opposite Matrigel, and the incidence of responding tumor formation was increased from 20% to 100% of injected mice (FIG. 1C). We confirmed that instigating cells did not metastasize to contralateral sites of responding cell implantation and that the neoplastic cells in the responding tumors were derived exclusively from the injected responder cells (FIG. 8D,E).

We also determined that instigating tumors established a supportive systemic environment within 30 days of implantation. Thus, when responding tumor cells were implanted following 30 days of BPLER tumor growth, the first palpable responding tumors were already evident 5 days later (FIG. 1D) and thus did not exhibit the 40-day lag period observed when the two cell lines were injected simultaneously (FIG. 1B). The average mass of these responding tumors was ˜ten-fold greater than that of the responder cell plugs recovered opposite Matrigel (FIG. 1E). Moreover, the presence of contralateral instigating tumors increased the incidence of responder tumor formation from 25% to 80%. Notably, instigating tumors were only ˜2 mm in diameter when the responder cells were injected (FIG. 8B). Taken together, these findings indicated that the instigating tumors acted in a systemic fashion to accelerate the growth of distant, otherwise-indolent responding tumor cells.

We next asked whether the capacity for systemic instigation was unique to the initially used BPLER cell line. We found that the MDA-MB-231 human breast cancer cells (231) also instigated growth of HMLER-HR responding tumors that were >25-fold larger than those injected opposite Matrigel (FIG. 1F); as before, the instigating tumor cells did not migrate to sites of responder cell injection (FIG. 8E). In contrast, three vigorously growing cell lines—PC3 human prostate carcinoma, murine Lewis lung carcinoma (LLC), and EpH4Ras murine mammary carcinoma—all failed to instigate responder tumor growth (FIG. 1F). Hence, the ability to systemically instigate indolent tumor outgrowth was uncoupled from the ability of transformed cells to form vigorously growing primary tumors.

Histopathological examination of tissues recovered from responding cell injection sites located opposite Matrigel plugs revealed a high degree of necrosis (FIG. 8C). Responding tumors growing opposite instigating BPLER or MDA-MB-231 tumors, however, closely resembled adenocarcinomas (FIG. 8C). The latter observations indicated that instigating tumors have a profound systemic effect on both the viability and histopathology of distantly located responding tumors.

### Example 2

### Incorporation of Bone Marrow-Derived Cells into Instigated Tumors

Bone Marrow Harvest and Transplantation

BMCs were harvested from donor mice by isolating and flushing the femurs with sterile Hanks' balanced salt solution (HBBS; Gibco) with penicillin/streptomycin/fungisone. Cells were washed 2× with sterile HBBS, dissociated with 18-gauge needles and passed through 70 μm nylon mesh filters. For transplantation experiments, 2×10⁶ BMCs from Rag1^{-/-}xEGFPTg donor mice were injected into the retroorbital sinus 8-10 h after irradiation of recipient mice (600 rads for Nude mice; 350 rads for NOD-SCID mice). Antibiotics were added to drinking water for 14 d following the procedure. At the end of each experiment, recipient mice were anesthetized by i.p. injection of Avertin and vasculature was exsanguinated by perfusion of sterile PBS through the left ventricle.

Results

As described above, the vigorous growth of responding tumors occurred only when the responder cells were implanted contralaterally to instigators. This fact, taken together with reported observations that certain types of stromal cells have origins in the bone marrow (Direkze and Alison, 2006), caused us to speculate that instigating tumors stimulated responding tumor growth by mobilizing stromal cell precursors from the bone marrow, thereby making them available via the circulation for recruitment by responding tumors.

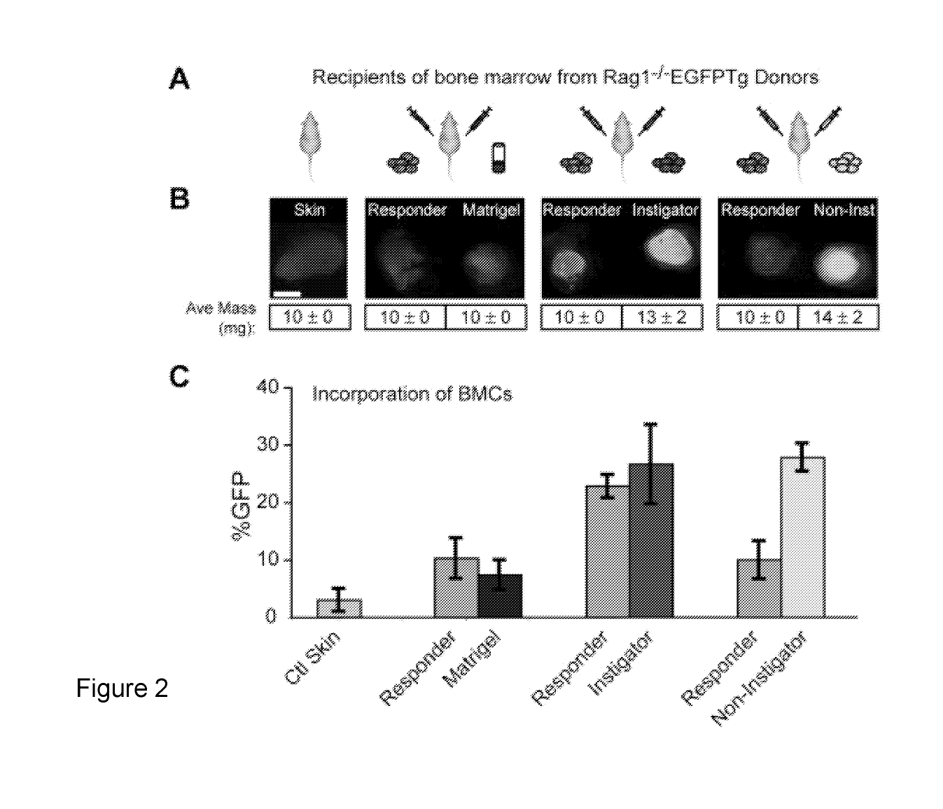

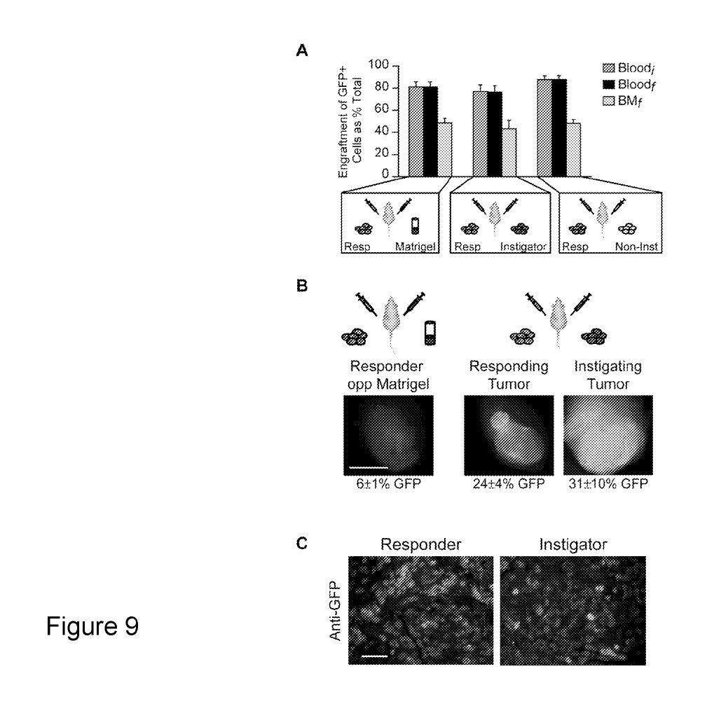

In order to determine whether the bone marrow was indeed involved in systemic instigation, we transplanted bone marrow cells (BMCs) from immunocompromised mice that ubiquitously express enhanced green fluorescent protein (Rag1^{-/-}EGFPTg mice) into irradiated mice that were used as hosts in subsequent experiments. After confirming successful engraftment of GFP+BMCs (FIG. 9A), various human tumor cells were implanted subcutaneously into these mice using the contralateral injection protocol (FIG. 2A). Small responder cell plugs that are implanted opposite non-instigating PC3 tumor cells can be recovered within ˜4 weeks of their initial implantation, but beyond a period of 4 weeks, these non-instigated cells are no longer visible at the injection site (unpublished observation). Consequently, for the purposes of these experiments, responder growths were compared for GFP⁺ BMC incorporation 4 weeks after tumor cell injections, a time when all xenografted masses were of comparable size (˜10 mg; ˜0.04% total body weight; FIG. 2B); thus, we ensured that any differences that we might observe in BMC recruitment were not due to differences in tumor size.

Incorporation of GFP+BMCs into responder grafts injected opposite Matrigel (10±4%) were not statistically significantly greater than those of control subcutaneous tissues taken from mice that had been transplanted with GFP+BMCs but had not been injected with tumor cells (3±3%; FIG. 2C). Moreover, in spite of the significant recruitment of GFP+BMCs to non-instigating PC3 tumors (28±2%), contralateral responding grafts failed to incorporate GFP+ cells to any extent above that of controls (10±3%; FIG. 2C). In contrast, GFP+ cells accounted for ˜23% of the total cells present in the responding tumors growing opposite BPLER instigating tumors, which were comprised of ˜27% GFP+ cells (FIG. 2C). Hence, instigation of responding tumor growth was accompanied by heightened recruitment of BMCs to the stroma of these tumors.

We also analyzed incorporation of GFP+ BMCs into tumors that had grown as xenografts for 9 weeks in such host mice. The responding tumors were comprised of ˜24% GFP+ BMCs, while the contralateral instigating tumors consisted of ˜31% GFP+ BMCs (FIG. 9B,C).

These observations suggested that instigating tumors stimulated responding tumor growth by mobilizing BMCs that were subsequently incorporated into the responding tumor stroma. Moreover, the fact that certain non-instigating tumors, despite their own rapid growth, were unable to mobilize BMCs to the stroma of the contralateral responders, suggested that only instigating tumors can actively communicate with the host bone marrow to provide the distant, otherwise-indolent tumors with stromal cells that they require for growth.

### Example 3

### Instigating Tumors Functionally Activate the Bone Marrow

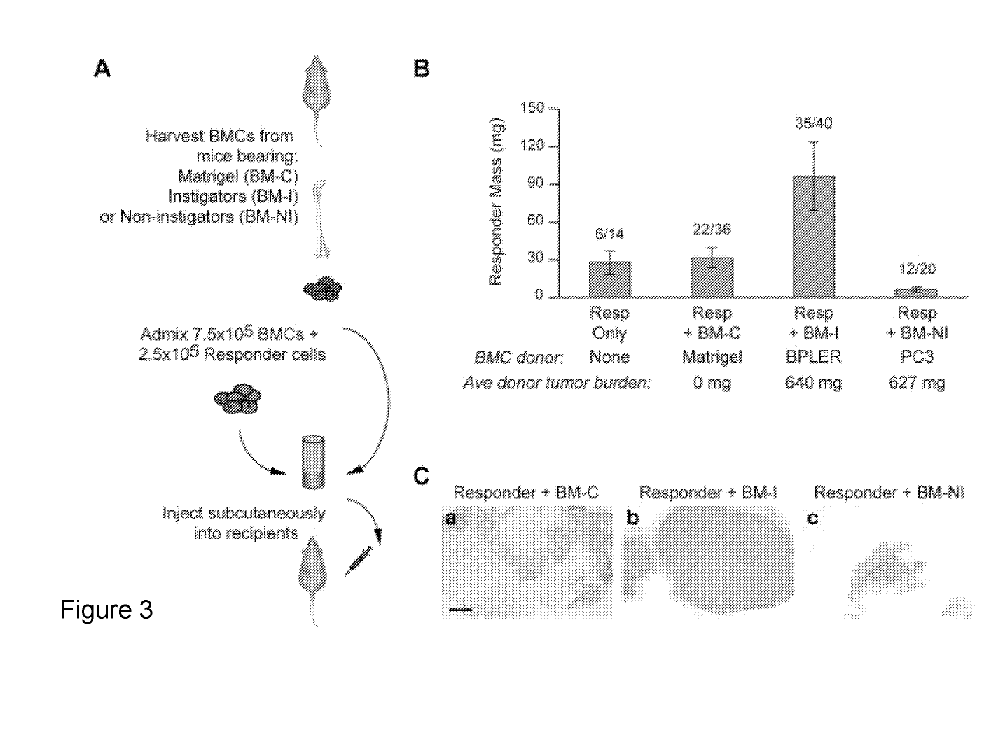

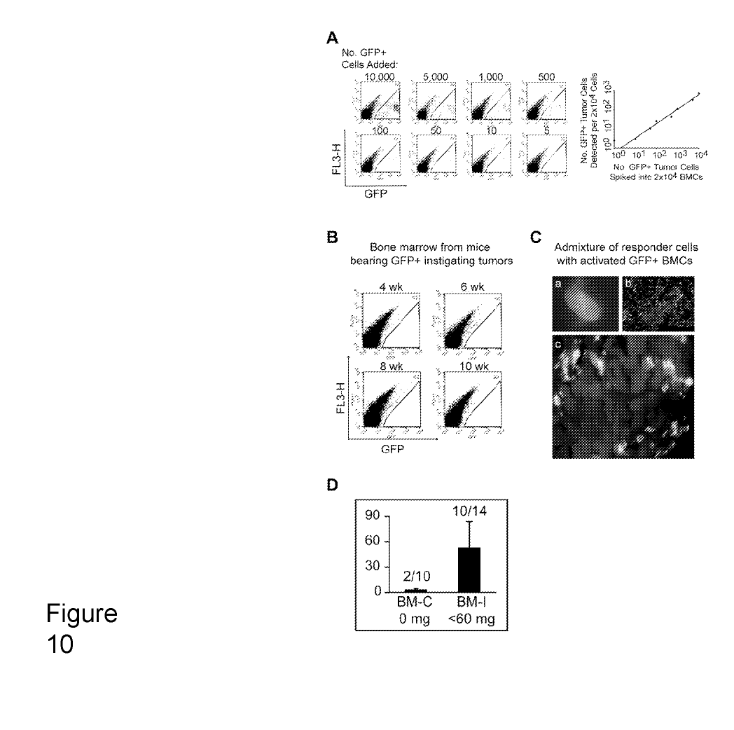

We devised a test to determine whether there were ascertainable functional differences between the bone marrow cells of various types of tumor-bearing hosts. Thus, we isolated BMCs from mice bearing either instigating BPLER tumors or size-matched, non-instigating PC3 tumors and mixed each of these BMC populations with responder cells prior to injection (FIG. 3A). As controls, we injected responder cells alone or, alternatively, mixed responder cells with BMCs from mice bearing only Matrigel plugs. We then gauged the relative abilities of these various admixed BMC populations to promote responder tumor growth over a period of 12 weeks. We also confirmed that instigating tumor cells were absent from the BMC populations used in these experiments (FIG. 10A,B).

When mixed with responder cells, BMCs from control, non-tumor bearing mice (BM-C) and from mice bearing non-instigating PC3 tumors (BM-NI) both failed to enhance responder tumor growth to any significant extent (FIG. 3B). Histological observation of the resulting masses revealed either large necrotic areas or only small clusters of apparently viable cells (FIG. 3C a,c). However, admixture of BMCs from mice bearing ˜640 mg BPLER instigating tumors (BM-I) greatly enhanced both the size and incidence of responder tumor formation (FIG. 3B); these tumors formed with a histopathology that closely mirrored the appearance of responding tumors growing opposite to instigating tumors (FIG. 3Cb). Moreover, mixing BMCs from instigator-bearing Rag1^{-/-}EGFPTg mice with responder cells, performed in a separate set of experiments, revealed that admixed GFP+ marrow-derived cells were retained within the responding tumor stroma after a growth period of 9 weeks (FIG. 10C).

We also functionally tested BMCs prepared from mice bearing relatively small instigating BPLER tumors (20-60 mg) and found that even these admixed BMCs enhanced responding tumor formation compared with control BMCs (FIG. 10D). These results indicated that instigating tumors as small as 20 mg—only ˜0.08% of total body weight—are capable of functionally activating the bone marrow and extended our earlier results by demonstrating that instigating tumors established a tumor-promoting systemic environment relatively early in their growth. Moreover, since the admixed BMCs from instigator-bearing mice recapitulated the effects of an implanted instigating tumor, we concluded that most if not all of the tumor-promoting effects of systemic instigation are achieved via endocrinal perturbation of the host bone marrow.

### Example 4

### Instigators Perturb the Bone Marrow Primitive Hematopoietic Cell Compartment

Flow Cytometric Analysis

Freshly harvested tissues were minced with scalpel blades and digested in 1 mg/ml collagenase A for 3-4 h at 37° C. with continuous rotation. Resulting cell suspensions were dispersed with an 18-gauge needle, washed 2× with Resuspension Buffer [2% heat-inactivated fetal calf serum in sterile HBBS] and passed through 70 μm nylon mesh filters. BMCs were harvested as described above. Tissue cells or BMCs were labeled for flow cytometry by incubation with appropriate antibodies for 30 min to 1 hr at 4° C. with continuous rotation. Antibodies used: PE-anti-Ly-6A/E/Sca-1 clone E13-161.7 (400 ng/10⁶ cells), APC-anti-CD117/c-Kit 2B8 (400 ng/10⁶ cells), and Mouse lineage panel kit (BD Pharmingen).

Results

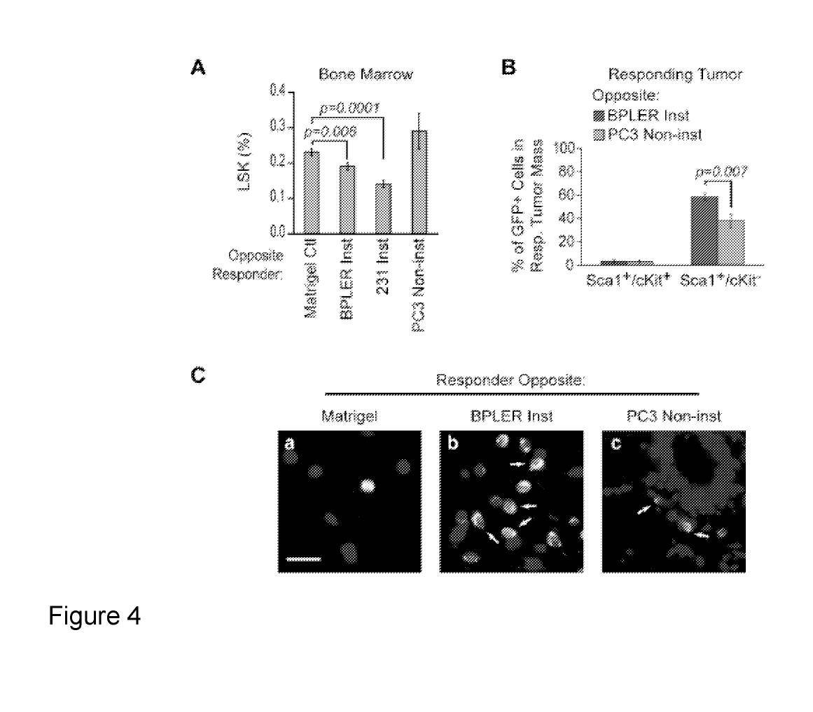

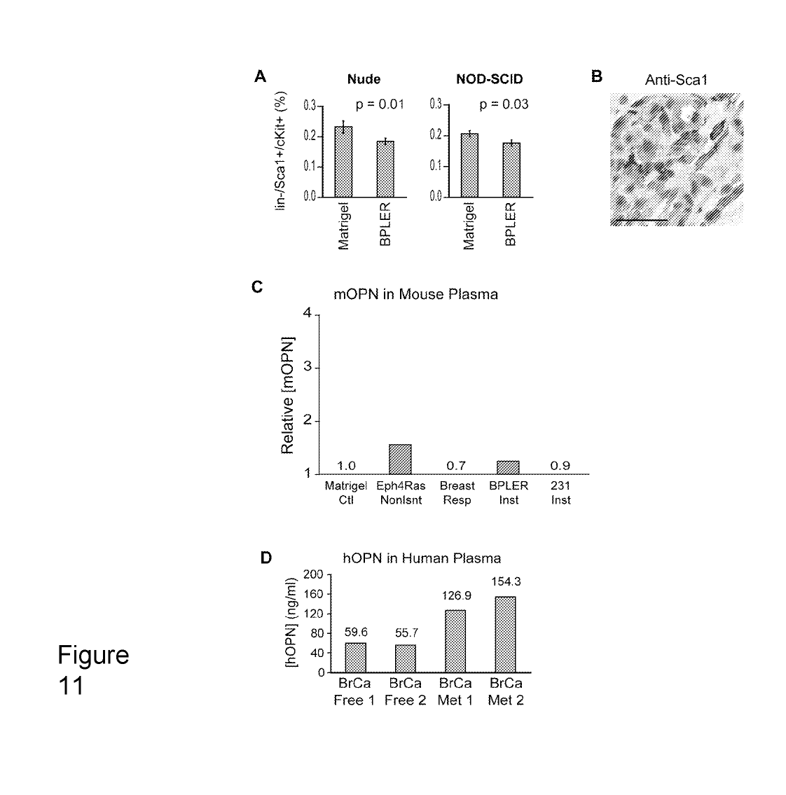

In order to determine how instigating tumors might affect bone marrow physiology, we used flow cytometric analysis to characterize the hematopoietic cell types in the marrow of host mice. We detected no significant changes in the representation of cell types in the marrow of the instigator-bearing mice, as compared to Matrigel-bearing mice, when we examined a number of BMC cell-surface antigens, including CD11b, CD45, Grl, VEGFR1, VEGFR2 and CD31 (not shown). However, we repeatedly observed a subtle yet significant decrease in the frequency of Lin⁻/Sca1⁺/cKit⁺ (LSK) cells in the marrow of mice bearing instigating tumors when compared with mice bearing only Matrigel plugs (FIG. 4A). Such LSK cells have been shown by others to represent the hematopoietic stem cells in mice (Kondo et al., 2003). This decrease in the LSK population was not manifest in the bone marrow of mice bearing non-instigating PC3 tumors (FIG. 4A). In separate experiments, we saw similar reductions in the LSK population in the bone marrow of two different strains of mice bearing BPLER instigating tumors when compared with mice injected only with Matrigel (FIG. 11A).

Bone marrow-derived Sca⁺ hematopoietic cells have been observed within primary tumor stromata and are thought to play a role in promoting tumor progression by mediating angiogenesis (Kopp et al., 2006). Accordingly, we undertook to determine whether the observed decrease in LSK cells in the bone marrow correlated with the presence of Sca⁺/cKit+ hematopoietic cells in the responding tumor stroma.

To do so, we measured the cell-surface expression of Sca1 and cKit on the GFP+ bone marrow-derived cells present in 4-week responding tumor masses that were implanted contralaterally to either BPLER instigators or PC3 non-instigators. The contribution of primitive Sca1⁺/cKit⁺ cells to the total GFP+ bone marrow-derived responder stromal compartment was minimal and equivalent between the two groups (˜3%; FIG. 4B). We found, instead, that the majority of the GFP+/Sca+ bone marrow-derived cells were negative for cKit and that these Sca1⁺/cKit⁻ cells comprised ˜66% of the GFP+ cells recruited to the responding tumors that had grown opposite instigating tumors; the GFP+ cells from indolent cell clusters recovered opposite non-instigators contained only ˜38% of these Sca1⁺/cKit⁻ cells (FIG. 4B). Cells in the bone marrow that are Sca1⁺/cKit⁻ have been described as a quiescent population of hematopoietic progenitor cells that are resistant to the cytotoxic effects of 5-FU and, under certain conditions, have reconstitution capability (Klarman, et al., 2003; Randall and Weissman, 1998).

Upon immunohistological examination of responding tumors for the co-localization of GFP and Sca1 antigens, we found that bone marrow-derived GFP+/Sca1⁺ cells (which were almost entirely cKit−, see above) were rarely observed in the responder cell plugs recovered opposite Matrigel or non-instigating tumors and, when present, were localized largely near blood vessels of the normal skin (FIG. 4C a,c). In contrast, when growing opposite instigating tumors for either 4 or 8 weeks, bone marrow-derived GFP+/Sca1⁺ cells were found intermingled with the neoplastic HMLER-HR responding cells FIGS. 4Cb and 11B). Collectively, these results demonstrate that instigating tumors specifically decrease the primitive hematopoietic cell compartment (LSK) in the host marrow. This perturbation is not associated with a concomitant recruitment of Sca1⁺/cKit⁺ cells to the responding tumor stroma but, rather, correlates with enhanced recruitment of Sca1⁺/cKit⁻ cells into the responding tumor stroma.

### Example 5

### Tumor-Derived Osteopontin is a Necessary Component of Systemic Instigation

ELISA

Mouse plasma was prepared by centrifugation of whole blood collected into EDTA Microtainer tubes (BD Pharmingen). Human blood samples were collected in red-topped vacutainer tubes; plasma was processed within 1 hr of collection and frozen at −80° C. in 1 ml aliquots; samples were thawed once and provided to researchers without patient identifiers. Human osteopontin levels were measured using species-specific TiterZyme EIA (Assay Designs) kits according to manufacturer's instructions.

OPN shRNA Plasmids

Five sequence-verified shRNA clones specific to human osteopontin were provided in the pLKO.1-Puro lentivirus expression plasmid (Mission shRNA; Sigma, St. Louis, Mo.). Infection and puromycin selection of target cells was performed as previously described (Stewart et al., 2003). Only those hairpins that suppressed OPN expression effectively without altering cell growth or morphology were used in subsequent experiments.

Design of shRNA for Osteopontin

The accession number for the cDNA used to design the interfering RNA sequences is NM_{—}000582 (SEQ ID NO:1). The sequence associated with NM_{—}000582 available at the effective filing date of this application is incorporated by reference herein. Exemplary shRNA sequences effective in suppressing the activity of osteopontin includes the following:

(SEQ ID NO: 3)
CCGGCCACAAGCAGTCCAGATTATACTCGAGTATAATCTGGACTGCTTG
TGGTTTTT
(SEQ ID NO: 4)
CCGGCCGAGGTGATAGTGTGGTTTACTCGAGTAAACCACACTATCACCT
CGGTTTTT

Results

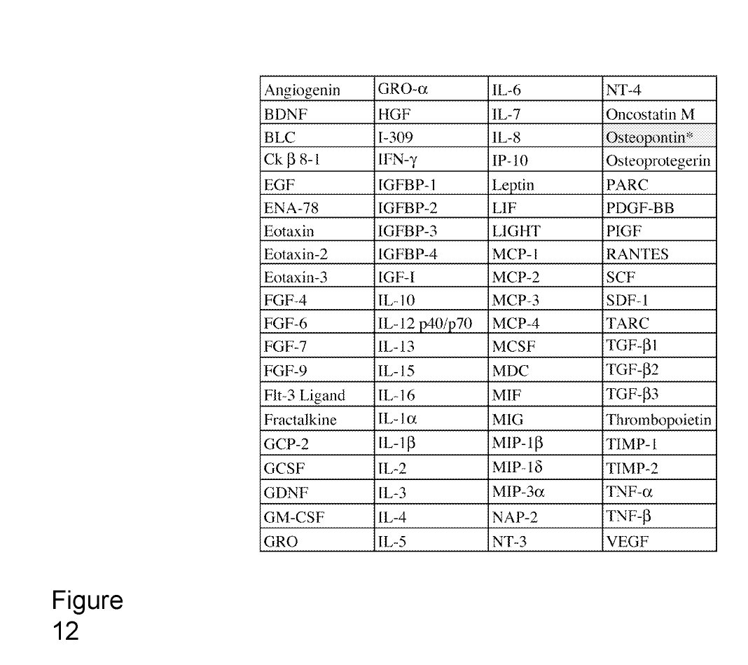

We wished to learn how instigating tumors communicate with and functionally perturb the host bone marrow. We therefore analyzed the plasma of mice bearing instigating tumors, non-instigating tumors, or Matrigel control plugs in order to determine whether we could detect alterations in the levels of various human cytokines that were secreted by the engrafted tumor cells. We tested 80 distinct human cytokines by either antibody array or ELISA (FIG. 12).

There were no significant differences in the plasma levels of these human cytokines in mice bearing instigating tumors compared with those bearing non-instigating tumors (not shown), with one exception—osteopontin (hOPN). In an initial experiment, plasma levels of tumor-derived hOPN in mice bearing instigating tumors were elevated ˜3-fold relative to non-instigating tumors (not shown).

Osteopontin (OPN) is a secreted glycoprotein having pleiotropic effects on inflammation, angiogenesis, fibrosis, and tumor metastasis and carries functional domains that are conserved across mouse and human species. (Cook et al., 2005; Rittling and Chambers, 2004). A number of studies have shown that OPN is secreted in a soluble form by a variety of tumor cell types and is elevated in the blood of many cancer patients with metastatic disease (Chatterjee and Zetter, 2005; Mor et al., 2005; O'Regan and Fleming, 2002; Rittling and Chambers, 2004; Rudland et al., 2002; Tuck and Chambers, 2001); however, such studies have not revealed a physiologic role for OPN in tumor pathogenesis.

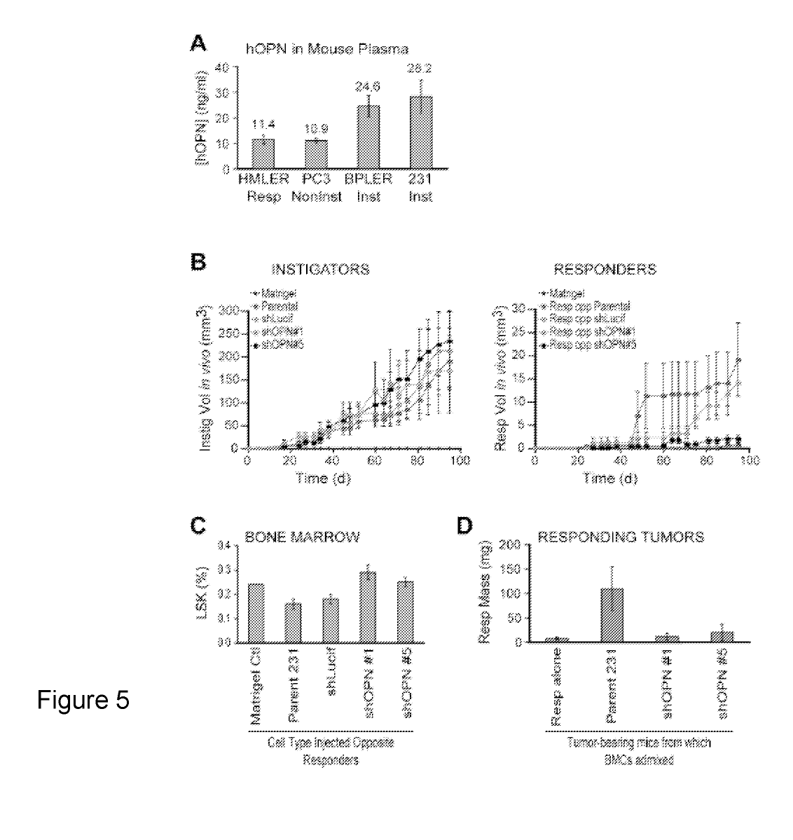

We therefore expanded our analysis of plasma hOPN to include mice bearing various instigating and non-instigating tumors. Plasma levels of tumor-derived hOPN in mice bearing instigating tumors—BPLER and MDA-MB-231—were elevated at least 2.2-fold, and up to 2.6-fold, above those of control mice injected with either HMLER-HR responders or non-instigating PC3 tumors (FIG. 5A). In contrast, there were no significant differences in plasma hOPN levels between mice injected with the responding cells and those injected with PC3 non-instigating tumors (FIG. 5A). We also determined that none of the tumor types that we implanted altered the plasma levels of murine osteopontin (mOPN) in host mice (FIG. 11C).

The relative increases in plasma hOPN that we observed in instigator-bearing mice were comparable to increases that we observed in the plasma of patients with metastatic breast cancer compared to cancer-free subjects (2.2-2.8-fold; FIG. 11D). Indeed, these elevations are consistent with previously published reports and are considered to be a clinically significant parameter that is associated with reduced patient survival (Furger et al., 2001; Singhal et al., 1997).

OPN is reported to restrict primitive hematopoietic cell (i.e., LSK) numbers in the bone marrow stem cell niche through its actions on the hematopoietic cell niche as well as on the LSK cells themselves (Nilsson et al., 2005; Stier et al., 2005); this was reminiscent of the decline in LSK numbers we observed in the marrow of mice bearing instigating tumors (FIG. 4A). Furthermore, hematopoietic stem cells express the OPN receptors, CD44 and α4 integrin, and the differentiation status of these cells can be altered by OPN signaling (Iwata et al., 2004; Schmits et al., 1997; Scott et al., 2003).

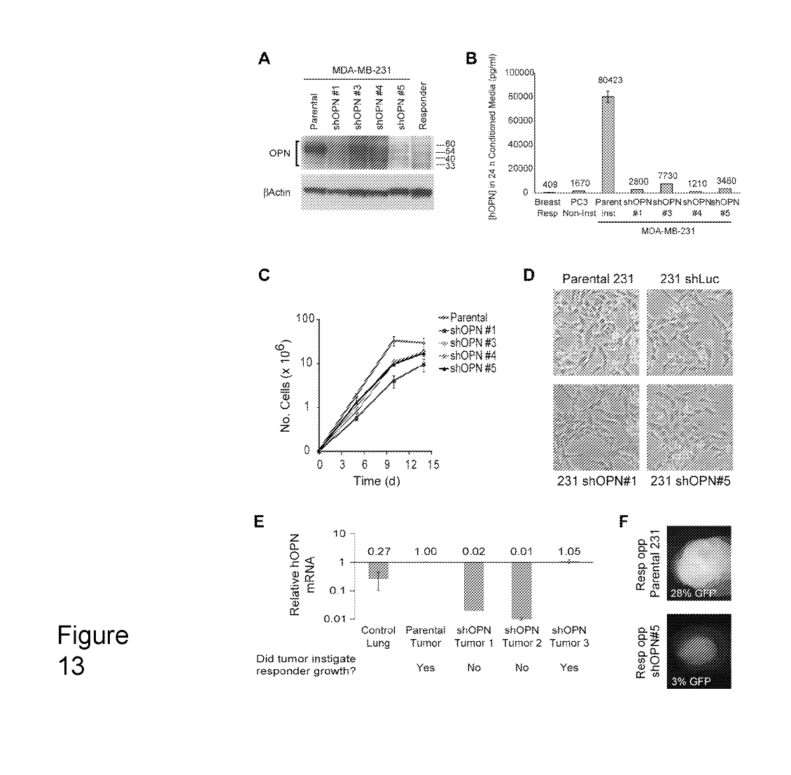

We therefore tested the notion that the circulating hOPN released by instigating tumors perturbs the host bone marrow, thereby facilitating systemic instigation. In order to do so, we suppressed hOPN expression in MDA-MB-231 (“231”) instigating tumor cells using shRNA; such suppression would presumably allow us to determine whether OPN deficiency would affect their ability to function as instigators. We identified two shRNA constructs—shOPN#1 and shOPN#5—that reduced OPN secretion at least 23-fold (by ELISA) and did not significantly alter either the morphology or growth kinetics of these cells in culture (FIG. 13A-D).

We injected either 231 cells or their OPN-deficient derivatives contralaterally to HMLER-HR breast responder cells in order to gauge the ability of these 231 variants to function as instigators. We observed that OPN deficiency did not impair in vivo growth kinetics of implanted instigating 231 cells in which the shRNAs were expressed (FIG. 5B, left; FIG. 13E). As expected, the parental 231 tumors, as well as control tumors expressing shRNA against luciferase, instigated growth of contralateral responding tumors (FIG. 5B, right). In striking contrast, responding tumors did not grow when the contralateral 231 tumors were deficient for OPN (FIG. 5B, right). Hence, despite the fact that OPN-deficient 231 cells continued to form vigorously growing tumors, OPN deficiency abolished their instigating ability.

We used mice equivalently engrafted with GFP+BMCs to determine whether suppressing OPN in instigating tumors had any effect on incorporation of BMCs into the contralateral responder cell plugs. Responder tumors grown opposite the parental 231 instigators were comprised of ˜28% bone marrow-derived cells, while responder cell plugs recovered opposite shOPN#5 tumors contained only ˜3% GFP+ bone marrow-derived cells (FIG. 5D).

The reduction in LSK cells that was reproducibly observed in the mice bearing parental 231 instigating tumors was absent in mice bearing OPN-deficient 231 tumors (FIG. 5C). Moreover, when we mixed responding BMCs from mice bearing OPN-deficient 231 tumors with responding cells prior to injection, responding tumor growth was not facilitated, whereas the admixed BMCs from mice bearing the parental 231 tumors, as before, supported tumor growth (FIG. 5D). These results indicate that instigating tumor-derived OPN is important to perturb the host bone marrow in order to mediate systemic instigation.

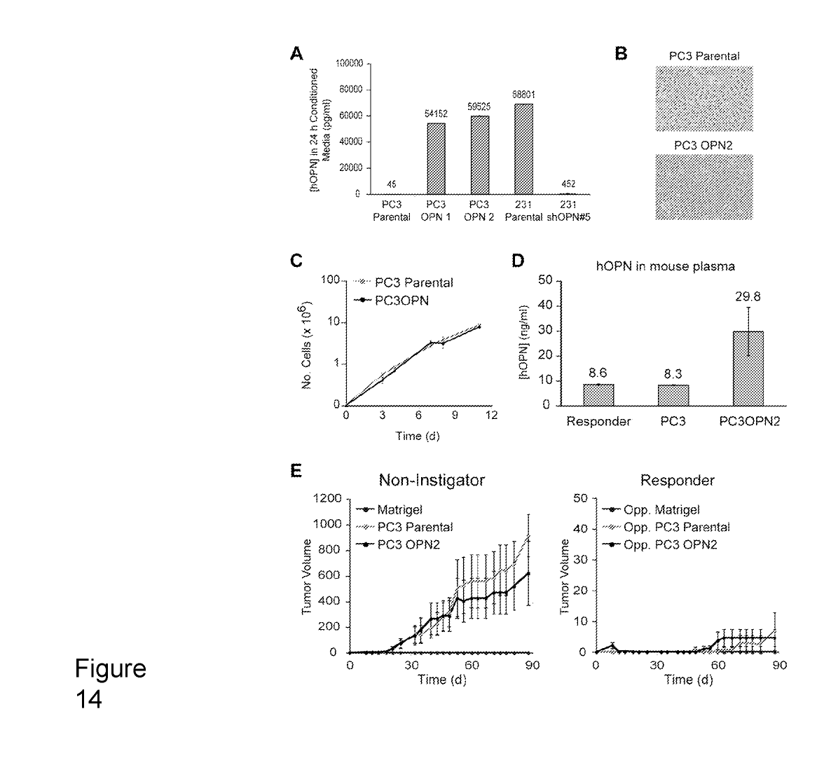

Finally, we expressed hOPN in the non-instigating PC3 cells, which normally secrete hOPN to negligible levels (FIG. 13B, 14A), in order to determine whether hOPN expression might confer upon these cells an ability to systemically instigate responder growth. Accordingly, we identified a population of PC3OPN cells that secreted hOPN at levels comparable to those of the instigating 231 cells and demonstrated growth kinetics that were no different from the parental PC3 cells in vitro (FIG. 14A-C). These PC3OPN cells were then implanted into host mice contralaterally to the responder cells. Although hOPN concentrations in the plasma of mice bearing the PC3OPN tumors were comparable to those of instigator-bearing mice (FIG. 14D), and both PC3 parental and PC3OPN tumors grew with similar kinetics in vivo (FIG. 14E, left), the PC3OPN tumors, like the PC3 parental tumors, did not instigate outgrowth of the contralateral responder cells (FIG. 14E, right). Collectively, these results reveal that OPN secretion is necessary, but not sufficient, for systemic instigation.

### Example 6

### Systemic Instigation of Metastatic Outgrowth

Analysis of Lung Metastases

Entire lungs were harvested, placed directly into sterile HBS, immediately examined under a dissecting microscope with fluorescence capability, and GFP⁺ surface foci were counted. Discreet GFP⁺ foci that were visible under 4× magnification were counted as micrometastases; GFP⁺ foci that were visible by eye (without requiring magnification) were counted as macrometastases.

Results

These observations indicated that primary tumors that secrete OPN are capable of systemically instigating outgrowth of distant indolent cells and suggested that mechanisms of systemic instigation might also serve to facilitate the outgrowth of disseminated metastatic cells. To test this hypothesis, we used a model of experimental metastasis to gauge the effects of systemic instigation on this process.

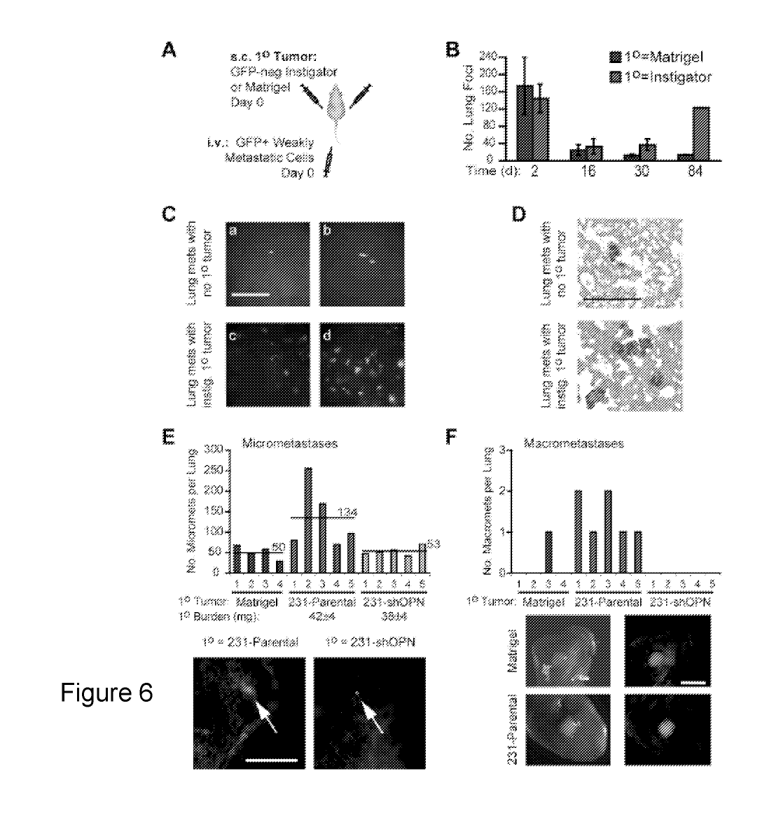

We simultaneously injected GFP-negative instigating BPLER tumor cells into subcutaneous sites and GFP+MDA-MB-231 cells (231+GFP) into the tail veins of non-irradiated mice (FIG. 6A). These 231+GFP cells, which serve as responders in these experiments, are only weakly metastatic and nearly 90% of the cells are cleared from the lungs of host mice within 1 day of intravenous injection (unpublished observations). At various time points during the subsequent 84 days, we examined the lungs of these hosts for the presence of GFP+ metastatic foci.

There was no significant difference between mice bearing subcutaneous instigating tumors and those bearing only Matrigel plugs when the numbers of 231+GFP micrometastatic lung foci were counted at days 2 and 16 following injections (FIG. 6B). Furthermore, the numbers of lung metastases decreased between days 2 and 16 in both groups, even though instigating tumors had started to grow within 16 days (FIG. 6B). These results suggested that the presence of a growing instigating tumor at a distant site did not affect the initial survival or retention of the weakly metastatic cells in the lungs of host mice.

In stark contrast, at the 30- and 84-day time points, the numbers of 231+GFP foci in the lungs of mice bearing primary instigating tumors were elevated 3- and 9-fold, respectively, over the basal level of foci observed in lungs of control mice (FIGS. 6B,C and 14A). Importantly, no instigating BPLER cells were observed in these instigated lung foci, indicating that the lung metastases were exclusively formed from the responding 231+GFP cells that had been injected intravenously (FIGS. 6D and 14B). Moreover, we observed Sca1+ cells intermingled with metastatic tumor cells only in the presence of subcutaneous instigating tumors (FIG. 14C). These data suggest that primary instigating tumors had a profound systemic effect on the outgrowth of otherwise-weakly metastatic cells that were present in the lungs.

Additionally, we observed Sca1+ cells in the stroma of the instigated metastatic outgrowths (FIG. 6D). In contrast, Sca1+ cells were not observed in the metastatic foci present in lungs of Matrigel-bearing mice (FIG. 6D), but instead, were restricted to the normal pulmonary vasculature (FIG. 14C), as previously reported by others (Kotton et al., 2003).

We next tested whether OPN was necessary for the effect of primary instigating tumors on the outgrowth of lung metastases in a model that mimics dissemination of metastatic cells from a primary tumor. In order to do so, we injected either Matrigel, the parental GFP-negative 231 cells, or GFP-negative 231-shOPN cells into subcutaneous sites and, at the same time, injected the weakly metastatic 231+GFP cells into the tail vein of hosts. All of these cells were derived from the same parental MDA-MB-231 cell line. Lungs were then examined 4 weeks later—a time prior to which parental 231 cells are capable of metastasizing to the lungs from subcutaneous sites (unpublished observations).

As before, the presence of subcutaneous instigating tumors enhanced the numbers of 231+GFP lung micrometastases nearly 3-fold when compared to lungs of mice bearing only Matrigel plugs at subcutaneous sites (FIG. 6E). Importantly, subcutaneous instigators in which OPN expression was suppressed lost their ability to enhance lung metastases (FIG. 6E). Moreover, macrometastases (foci capable of being observed by eye) were present in 100% of the lungs from mice bearing subcutaneous instigating tumors, while none of the mice bearing tumors deficient for OPN had macrometastatic tumors in their lungs (FIG. 6F). Importantly, the average primary subcutaneous tumor burden was approximately equivalent in both groups of mice; these tumors were small (˜40 mg) and represented ˜0.16% of total body mass (FIG. 6E).

These data demonstrate that secretion of OPN is necessary for the ability of instigating tumors to systemically instigate outgrowth of distant responding tumor cells, independent of whether they are implanted into subcutaneous sites or have arrived in the lungs via the circulation.

### Example 7

### Systemic Instigation of Human Colon Tumor Surgical Specimens

Human Colon Tumor Specimens

Fresh surgical specimens of patient tumors were obtained with patient consent from the Tissue Procurement Facility at Roswell Park Cancer Institute shortly after resection, and applied to the SCID mouse xenograft model as described (Hylander et al., 2005; Naka et al., 2002). The tumor specimen that was used as a responder in the bilateral instigation system was quickly thawed at 37° C., washed 2× with RPMI, cut into 1-2 mm segments and implanted beneath the skin of NOD-SCID mice.

Results

The present results demonstrate that actively growing tumors, even when relatively small, can exert systemic effects that are sufficient to induce distantly implanted, transformed cells to progress from an indolent state to one yielding vigorous tumor growth. Thus, we were curious if systemic instigation could be used to facilitate the growth of human tumor surgical specimens, which usually do not grow well as xenografts.

It was previously demonstrated that some surgical samples of human tumors are capable of growing as xenografts after serial passage through SCID mice (Hylander et al., 2005; Naka et al., 2002), while the majority of human tumors are incapable of doing so. Accordingly, in order to select a human tumor sample that would serve as an indolent responder in our systemic instigation model, we first screened a number of surgical samples for their growth as xenografts in SCID mice.

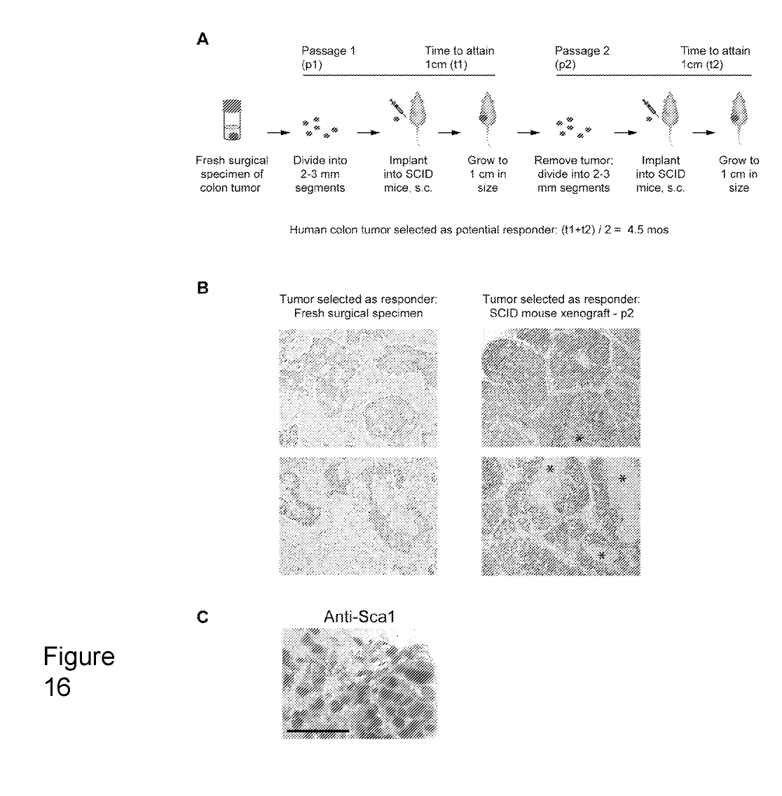

In all of these experiments, we prepared ˜2 mm fragments of tumors that were surgically removed from patients with colon cancer and implanted them beneath the skin of SCID mice (FIG. 16A). After 2 serial passages through the mice, we identified a patient tumor xenograft that exhibited slow growth kinetics and yielded histopathology involving widespread necrosis (FIG. 16B,C). This tumor sample was therefore designated as a candidate responder in our systemic instigation model. Accordingly, 1-2 mm fragments of this human colon carcinoma xenograft were applied to the bilateral instigation protocol and implanted subcutaneously into NOD-SCID mice contralaterally to either Matrigel or to instigating BPLER human mammary carcinoma cells.

Segments of the colon tumor that were implanted contralaterally to Matrigel were unable to grow during the 40 day experimental time period (FIG. 7A, left). In striking contrast, all of the colon tumor segments (3 of 3) implanted contralaterally to the BPLER instigators displayed robust growth kinetics; growth of these responding colon tumor specimens was first observed 27 days after implantation and lagged the first growth of the contralateral instigators by only 15 days (FIG. 7A, center). As a control, we determined that neither of the responder types—HMLER-HR responder nor colon surgical sample responder—could instigate the growth of the other when implanted contralaterally (FIG. 7A, right).

The responding colon tumors that had grown opposite instigating BPLER tumors displayed histopathology consistent with adenocarcinomas (FIG. 7B) and the neoplastic cells were indeed actively proliferating, as confirmed by staining for Ki67 (FIG. 7C). As before, Sca1+ cells were observed in these tumor stromata and were localized near areas of proliferating tumor cells (FIG. 16C).

These findings hold at least two implications: First, mammary tumors can act across tissue lineages to systemically instigate growth of colon tumors, indicating the generality of this physiologic signaling. Second, the presently described procedure, or derivatives thereof, can be used to study the growth of human tumor specimens and tumor cell lines that might otherwise grow very slowly or not at all as xenografts in vivo.

### Example 8

### Stromal Desmoplasia Arises as a Result of Systemic Instigation

As discussed above, we demonstrated that instigating mammary carcinoma tumors induce the growth of contralaterally implanted responding tumors. In this experiment, we wished to investigate whether the histopathology of the responding tumors was likewise affected. In particular, we examined the stromal compartment of responding tumors for evidence of stromal desmoplasia.

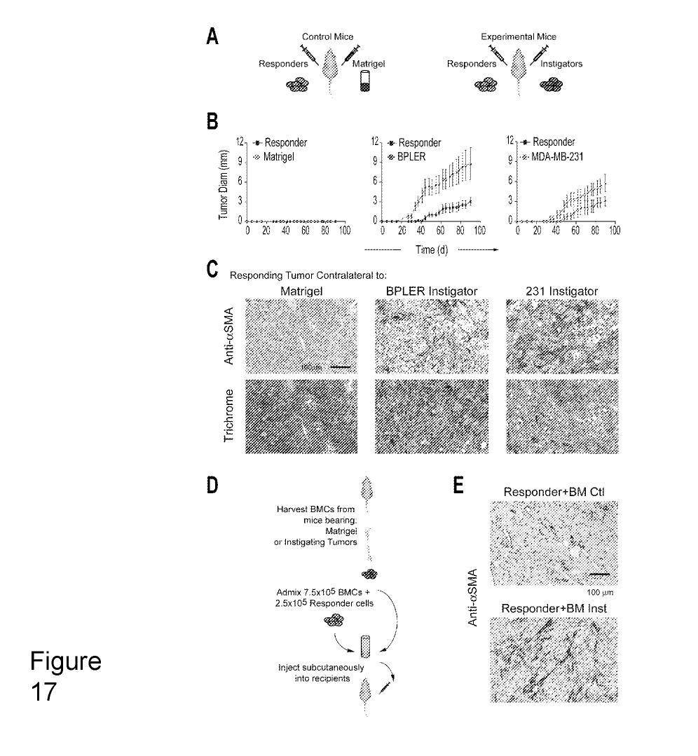

Accordingly, we introduced the human breast cancer cell lines—BPLER and MDA-MB-231 (231)—subcutaneously into the right flanks of Nude mice, while weakly tumorigenic, transformed mammary epithelial HMLER-HR cells (i.e., responders) were injected into the contralateral flanks of these mice as described in Example 1. As a control, Matrigel vehicle was injected contralaterally to the indolent responder cells in another group of mice (FIG. 17A).

When responder cells were implanted contralaterally to either BPLER or 231 cells, responding tumors demonstrated a lag phase of ˜40 days, after which they exhibited a constant, progressive increase in size (FIG. 17B). By contrast, the responding cells implanted contralaterally to Matrigel plugs failed to form externally measurable tumors (FIG. 17B); however, after surgical exposure of the subcutaneous space at the experimental end point, we could recover small masses in ˜20% of the injected mice.

We examined the resulting tumors for the presence of α-smooth muscle actin (αSMA)-positive myofibroblasts and collagen deposition, which are hallmarks of a reactive stroma (i.e., desmoplasia). Responding cell masses recovered from sites contralateral to Matrigel plugs displayed very little collagen deposition or αSMA expression (FIG. 17C); the majority of the αSMA-positive cells within these growths were also associated with expression of the mouse endothelial cell antigen (MECA32), suggesting that these cells were pericytes (Hirschi et al. (1996) “Pericytes in the microvasculature,” Cardiovasc Res. 32(4):687-98; Song et al. (2005) “PDGFRbeta+ perivascular progenitor cells in tumours regulate pericyte differentiation and vascular survival,” Nat. Cell Biol. 7(9):870-9) (not shown).

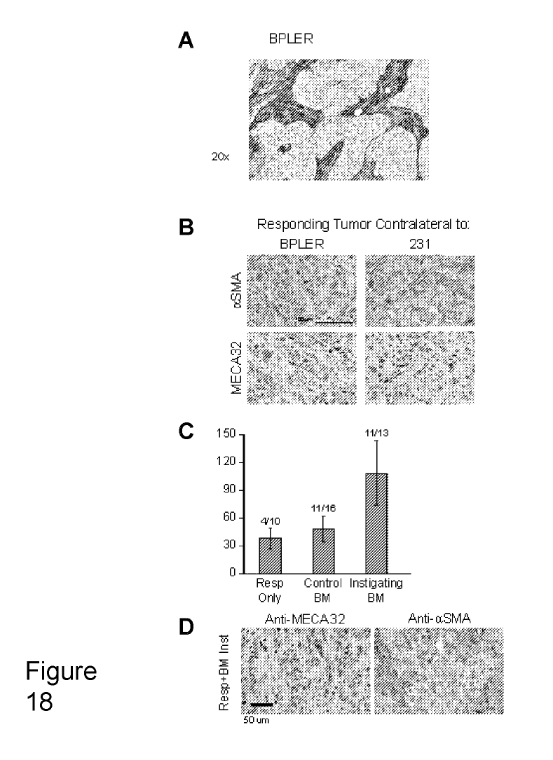

In striking contrast, αSMA-positive cells and collagen were distributed widely and uniformly throughout the responding tumors that were implanted contralaterally to either BPLER or 231 instigating tumors (FIG. 17C). Staining for αSMA in these tumors overlapped with staining for endothelial cells only to a minimal extent (FIG. 18B), suggesting that the majority of αSMA+ cells in these instigated tumors were myofibroblasts rather than pericytes. Such myofibroblast-rich, reactive stroma is almost always observed in malignant human adenocarcinomas and is associated with poor prognosis (Walker R A. (2001) “The complexities of breast cancer desmoplasia,” Breast Cancer Res. 3(3):143-5; Kalluri et al. (2006) “Fibroblasts in cancer,” Nat. Rev. Cancer 6:392-401). The instigating BPLER tumors also formed a desmoplastic stroma, as indicated by the presence of αSMA+ cells and collagen deposition spread widely and uniformly throughout these tumors (FIG. 18A).

We also observed that αSMA+ cells were present in the metastatic outgrowths in the lungs of mice bearing subcutaneous instigating BPLER tumors. This is contrary to what we observed in the lungs of control animals in which micrometastatic foci were devoid of αSMA+ cells.

Collectively, these results revealed that, in addition to effects on responding tumor growth, the instigating mechanism had a profound effect on the histopathology of responding tumors through its generation of stromal desmoplasia in these growths.

### Example 9

### Bone Marrow from Hosts Bearing Instigating Tumors Mediates Stromal Desmoplasia

As described above, we showed that bone marrow-derived cells were recruited into the responding tumor stroma as a consequence of bone marrow activation. Moreover, we determined that BMCs extracted from, for example, BPLER instigator-bearing mice, when mixed with responding tumor cells, stimulate the growth of responding tumors and thus mimic the effects of systemic instigation.

This response provided us with a functional test of the biological state of the admixed BMCs. In this experiment, we used this test to determine whether the stromal desmoplasia observed in the responder tumors was also mediated via the bone marrow of instigator-bearing animals. To do so, we mixed the responding cells with BMCs from mice bearing either Matrigel plugs (BM-C) or BPLER instigating tumors (BM-I) prior to implantation (FIG. 17D). Importantly, no tumor cells were detected in the bone marrow samples that were used in these experiments.

The admixed control BM from the Matrigel-bearing mice did not significantly enhance responding tumor size when compared with implantation of responder cells alone (FIG. 18C). Similar to our previous results, admixture of bone marrow cells from instigator-bearing animals increased the incidence of tumor formation and enhanced the size of those tumors that did form, when compared with the control BMC mixtures (FIG. 18C).

We found that the type of BMCs that were admixed profoundly influenced the histopathology of the responding tumors. When control BMCs from Matrigel-bearing mice were mixed with the responder cells, the resulting growths were devoid of desmoplastic stroma (FIG. 17E). In these small masses, αSMA+ cells were restricted to a small number of blood vessels, indicating that they were most likely pericytes (not shown).

In striking contrast, the presence of αSMA+ cells and collagen deposition occurred widely and uniformly throughout the responding tumors resulting from the mixture of BM-I with the responder cells (FIG. 17E). In these tumors, αSMA stained not only pericytes, but also the widely dispersed, abundant myofibroblasts (FIG. 18D). Importantly, the reactive tumor stroma resulting from admixture of BM-I cells closely resembled the stroma of responding tumors implanted opposite instigating tumors (FIG. 17C), indicating that most, if not all, of the effects of instigation of stromal desmoplasia was mediated via the actions of cells present in the bone marrow of instigating tumor-bearing mice.

### Example 10

### Activated Bone Marrow Cells from Instigator-Bearing Mice that Mediate Systemic Instigation

As described above, we determined that instigator-activated bone marrow is defined by a significant decrease in the frequency of Lin⁻/Sca1⁺/cKit⁺ (LSK) cells when bone marrow from instigator-bearing mice was compared with that of mice bearing non-instigating PC3 tumors or Matrigel plugs. Such LSK cells have been shown by others to be enriched for the hematopoietic stem cells of mice (Kondo et al., 2003).

We first examined more closely the nature of the bone marrow-derived cells that were recruited into various responding tumor stromata. To do so, we performed flow cytometric analysis of the cellular composition of responding tumors that had grown for 4 weeks opposite control Matrigel plugs, BPLER instigating tumors, or PC3 non-instigating tumors in mice that had previously undergone lethal irradiation and had been rescued by transplantation of green fluorescent protein (GFP) BMCs. We analyzed cells expressing various cell-surface markers, or combinations thereof, including: Sca1, CD11b/CD45, VEGFR1, VEGFR2, and CD31. Control experiments confirmed equivalent multi-lineage engraftment of GFP⁺ donor cells among all groups of recipient mice and indicated that the spectrum of BMC-derived mesenchymal cells in the tumor stroma was much different than the spectrum of such cells recruited to other tissues. Furthermore, all responding tumors that we analyzed were of equivalent mass; hence, we could ensure that any differences in BMC recruitment that we might observe would not be due to differences in tumor size.

Incorporation of three bone marrow-derived cell populations clearly distinguished the GFP⁺ stroma of responding tumors growing opposite instigators from that of all other groups of tumors; these were Sca1⁺, CD11b⁺/CD45⁺, and VEGFR1⁺ cells. There were significantly more of each of these cell types in the stroma of responding tumors opposite instigators than there were in the stroma of control tumors. In the bone marrow, Sca1⁺ is expressed by hematopoietic stem cells as well as other oligopotent cells. CD11b⁺/CD45⁺ double-positive cells represent hematopoietic cells of myeloid lineage (Forsberg et al. (2006) “Hematopoietic stem cells: expression profiling and beyond,” Stem Cell Rev. 2(1):23-30), and VEGFR1 (Flt-1) is expressed by a variety of cell types, predominantly cells of the myelomonocytic lineage that are largely involved in vascular remodeling (Rafii et al. (2002) “Contribution of marrow-derived progenitors to vascular and cardiac regeneration,” Semin. Cell Dev. Biol. 13(1):61-7).

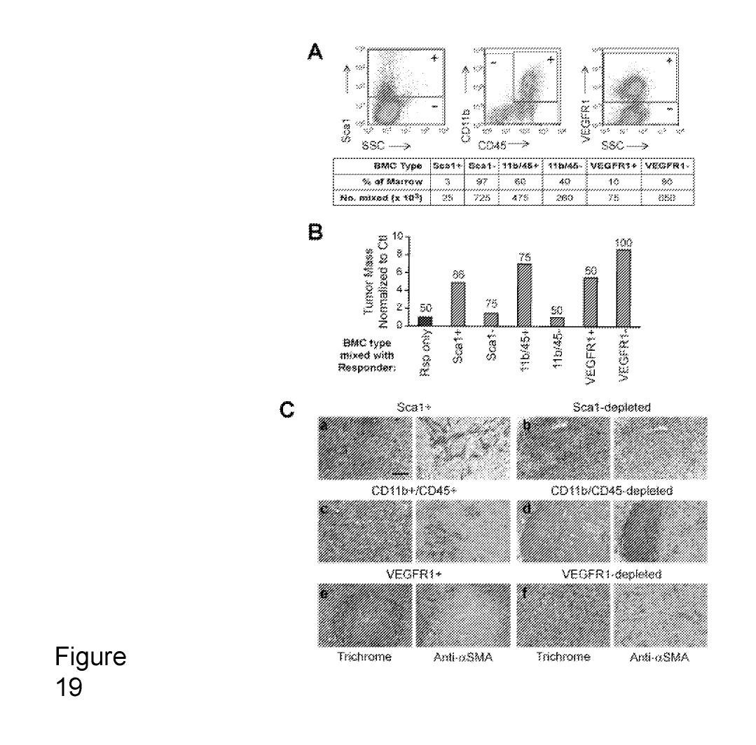

Consequently, we isolated by FACS the Sca1⁺, CD11b⁺/CD45⁺, and VEGFR1⁺ populations of BMCs, or the corresponding BMCs depleted of such cell populations, from bone marrow of mice bearing BPLER instigating tumors in an effort to identify cell type(s) in the bone marrow of these hosts that are responsible for systemic instigation (FIG. 3A MS2). We then mixed each BMC subpopulation with responder cells; the numbers of BMCs of each type that were admixed to responder cells (FIG. 19A) reflected the representation of that particular cell population in the whole marrow and was calculated as a percentage of the total number of BMCs (7.5×10⁵) that we had mixed in our earlier experiments. Accordingly, 2.5×10⁴ Sca1⁺ cells, 4.75×10⁵ CD11b⁺/CD45⁺ cells, or 7.5×10⁴ VEGFR1⁺ cells were mixed with 2.5×10⁵ responder cells and the mixtures were implanted into mice to determine whether any of these subpopulations could participate in the formation of tumor stroma and the acceleration of responding tumor growth.

Tumors resulting from the admixture of responder cells with either the Sca1⁺ or the CD11b⁺/CD45⁺ BMCs from mice bearing instigating tumors were at least 5-fold greater in mass and occurred with greater incidence than those of responding cells injected alone (FIG. 19B). These tumors yielded histopathology that most closely resembled responding tumors that arose opposite instigating tumors; specifically, these tumors all formed with a reactive stroma that was rich in αSMA+myofibroblasts and accompanying collagen deposition (FIG. 19D a,c).

The corresponding BMC populations that were depleted of either of these subpopulations (i.e., 7.25×10⁵ Sca1-depleted or 2.6×10⁵ CD11b/CD45-depleted populations) failed to enhance growth of responding tumors (FIG. 19B). Histological examination of the masses recovered from these admixtures demonstrated either vascularized, non-desmoplastic tumors or small benign cell clusters (FIG. 19D,b,d).

Tumor-promoting ability did not segregate with VEGFR1 expression, as both VEGFR1⁺ BMCs (7.5×10⁴ cells) and the corresponding VEGFR1-depleted (6.5×10⁵ cells) populations promoted responder growth to a similar extent; these tumors were, in both cases, at least 5.5-fold greater in mass than the control responder plugs (FIG. 19B). Moreover, the incidence of tumor formation was not enhanced by mixture of responder cells with VEGFR1′ BMCs (FIG. 19B) and these tumors formed with non-desmoplastic stroma (FIG. 19D,e). The admixed VEGFR1-depleted BMCs yielded responding tumor growth in 100% of mice injected (FIG. 19B) and these tumors formed with a desmoplastic stroma (FIG. 19D,f) similar to that we observed when responding tumors were implanted contralaterally to instigating tumors.

When considered collectively, these results indicated either that three different groups of cells in the instigator-activated bone marrow—Sca1⁺, CD11b⁺/CD45⁺, and VEGFR1-depleted—are each capable of instigating desmoplastic tumor growth, or that the activated subpopulation(s) is/are contained within each of these three populations. Significantly, Sca1⁺ cells were most potent in their instigation ability, as only 2.5×10⁴ of these cells achieved nearly equivalent responding tumor incidence, size, and stromal desmoplasia when compared to 4.75×10⁵ CD11b⁺/CD45⁺ cells (i.e., 19-fold more cells) and 6.5×10⁵ VEGFR1-depleted cells (i.e., 26-fold more cells).

Due to their potent tumor-promoting capabilities, we examined the contribution of the Sca1⁺ cells to the other instigating bone marrow cell types—CD11b⁺/CD45⁺ and VEGFR1-depleted cells—that had been identified in the marrow of mice bearing instigating tumors. We found that the CD11b⁺/CD45⁺ cell population contained ˜90% of the Sca1⁺ found in the marrow of these mice, while the VEGFR1-depleted cell population contained ˜95% of the Sca1⁺ population. Thus, we concluded that the biological activities (i.e., instigation of responding tumor growth and development of stromal desmoplasia) of the CD11b⁺/CD45⁺ and VEGFR1-depleted cell populations might be attributed to the presence of an instigating Sca1⁺ subpopulation.

### Example 11

### Sca1⁺/cKit⁻ BMCs from Instigator-Bearing Mice, but Not Control Mice, are Activated to Mediate Systemic Instigation

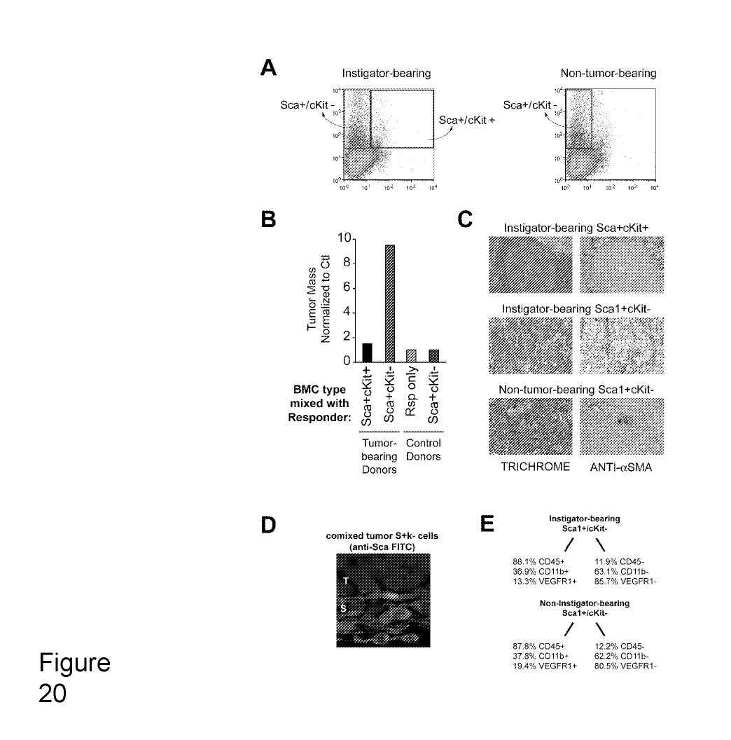

The tumor-promoting powers of the Sca1⁺ BMCs prepared from hosts bearing instigating tumors caused us to examine this population more closely. Thus, we further fractionated the Sca1⁺ BMCs from BPLER instigator-bearing mice into Sca1⁺/cKit⁺ and Sca1⁺/cKit⁻ subpopulations (FIG. 20A). We then mixed 7,500 Sca1⁺/cKit⁺ or 2×10⁴ Sca1⁺/cKit⁻ cells, (based on their representation among 10⁶ whole unfractionated bone marrow cells) with 2.5×10⁵ responder cells prior to injection into hosts.

The Sca1⁺/cKit⁺ cell fraction that was isolated from the bone marrow of instigator-bearing mice was not capable of enhancing responding tumor growth to any significant extent (FIG. 20B). In fact, the few tumor masses that we recovered from such cell mixtures showed non-desmoplastic stroma with areas of necrosis and edema (FIG. 20C,a).

In striking contrast, only 2×10⁴ Sca1⁺/cKit⁻ BMCs from instigator-bearing mice were able to instigate growth of responding tumors that were ˜6-fold larger than the tumors formed from mixtures containing Sca1⁺/cKit⁺ BMCs and ˜9-fold larger than masses formed from responding tumor cells implanted on their own (FIG. 20B). Moreover, these responding tumors formed with a desmoplastic stroma (FIG. 20C,b). We also determined that Sca1⁺ cells are indeed maintained in the stroma of responding tumors that grew as a result of these admixtures (FIG. 20D).

We next wished to determine whether the tumor-promoting activity of the Sca⁺/cKit⁻ population is unique to BMCs of instigator-bearing hosts, or whether such cells in the marrow of control mice might also have this activity. Accordingly, Sca1⁺/cKit⁻ cells were sorted from the bone marrow of mice bearing Matrigel plugs in an identical manner as those we had sorted from the marrow of the BPLER instigator-bearing mice (FIG. 20A). These cells (2×10⁴) were then mixed with the responders and implanted into host mice. We found that the Sca1⁺/cKit⁻ BMCs isolated from the control mice did not enhance responding tumor growth when admixed with the responding cells (FIG. 20B). The small resulting tumors formed with a non-desmoplastic, vascularized stroma without displaying edema or necrosis (FIG. 20C,c).

Taken together, these results revealed that (i) the Sca1⁺/cKit⁻ population from hosts bearing instigating tumors is highly enriched for the functionally activated cells that instigate responding tumor growth; and (ii) that cells of the Sca1⁺/cKit⁻ profile, although equally represented in number in the bone marrows of all groups of mice differed in their biological activity when isolated from the bone marrow of instigator bearing hosts compared to bone marrow of control hosts.

### Example 12

### Flow Cytometric Analysis of Sca1⁺/cKit⁻ Cells

Because of the significant difference in instigating activity between Sca1⁺/cKit⁻ BMCs from instigator-bearing mice and the antigenically identical cells from control mice, we wished to determine whether use of other potentially relevant known cell-surface markers would allow us to further subfractionate these cells. Hence, we performed flow cytometric analysis to compare the Sca1⁺/cKit⁻ cells from the bone marrow of mice bearing BPLER instigators with those of control mice. In this instance, we surveyed this population for the presence of the following cell-surface antigens: CD11b, CD45, VEGFR1 and GR1, all of which have been found to be displayed by cells known to participate in one way or another in fostering tumor growth (Direkze et al. (2006) “Bone marrow and tumour stroma: an intimate relationship,” Hematol. Oncol. 24(4):189-95).

Flow cytometric analyses revealed no significant differences in the representation of cells bearing these other surface markers between the Sca1⁺/cKit⁻ populations of instigator and non-instigator tumor-bearing mice (FIG. 20E). In the bone marrow from both groups of mice, ˜88% of the activated Sca1⁺/cKit⁻ BMCs were positive for the CD45 marker (FIG. 20E), thus indicating that the majority of these cells are of hematopoietic origin. Additionally, ˜37% of the Sca1⁺/cKit⁻ were positive for CD11b in both groups of mice, indicating that some of these cells also derive from a myeloid subpopulation of the hematopoietic compartment. Finally, there were no significant differences between groups of mice in the numbers of Sca1⁺/cKit⁻ BMCs that expressed either VEGFR1 or Grl cell-surface antigens (FIG. 20E).

### Example 13

### In Vivo Growth Kinetics of Her2-Positive Cells

Female Nude mice were purchased from Taconic (Hudson, N.Y.); a colony of NOD-SCID mice was maintained in-house. All experiments were performed in accordance with regulations of MIT Committee on Animal Care protocol (1005-076-08). SKBR3 and BT474 cells were maintained under standard culture conditions. Tumor cells were suspended in 20% Matrigel (BD Biosciences, San Jose, Calif.) and injected contralaterally beneath the skin of non-irradiated recipient mice. Tumor diameter was measured on the flanks of live nude mice using calipers; volume was calculated as 4/3πr³. Cells were suspended in a total volume of 100 μl and implanted in the following numbers:

| Left Flank | Right Flank |
|---|---|
| 5 × 10⁵ SKBR3 | 2 × 10⁶ MDA-MB-231 |
| 2.5 × 10⁵ HMLER hygro-H-rasV12 | 5 × 10⁵ BT474 |

Human epidermal growth factor receptor 2 (Her2) is a tyrosine kinase receptor member of the ErbB family of proteins and has been demonstrated to promote tumorigenesis and tumor progression (Bargmann, C. I. and Weinberg, R. A.; 1988; EMBO-J 7:2043-52). Her2 is overexpressed in approximately 20-30% of breast cancer patients and is correlated with poor prognosis and reduced survival (Slamon, D. J., et al., 1987; Science 235:177-82; Owens, M. A., et al., 2004; Clin Breast Cancer 5:63-69; Ross, J. S., et al., 2003; Oncologist 8:307-25). Monoclonal antibodies have been used to treat breast cancer patients with Her2-positive disease, yet these treatments have met with limited success and it is thought that many tumors develop resistance to treatment (Nielsen, D. L, et al., 2008; Cancer Treat Rev, November (ahead of print)). Experimental study of Her2-positive tumors has been hampered by the lack of sufficient xenograft models; in many cases, experimentation relies on ectopic overexpression of the receptor in Her2-negative cell lines, which grow in host mice to a better extent than tumor cell lines that endogenously express Her2. Two cell lines are often used in xenograft studies: SKBR3 and BT474 cells. In particular, the SKBR3 cell line forms xenografted tumors with low incidence, and often these tumors grow to only a small size.

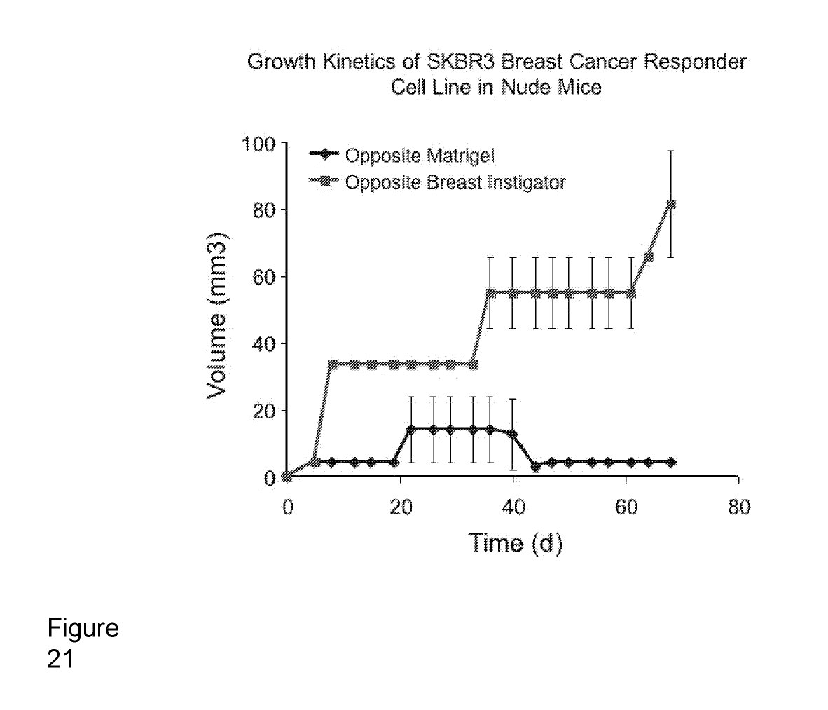

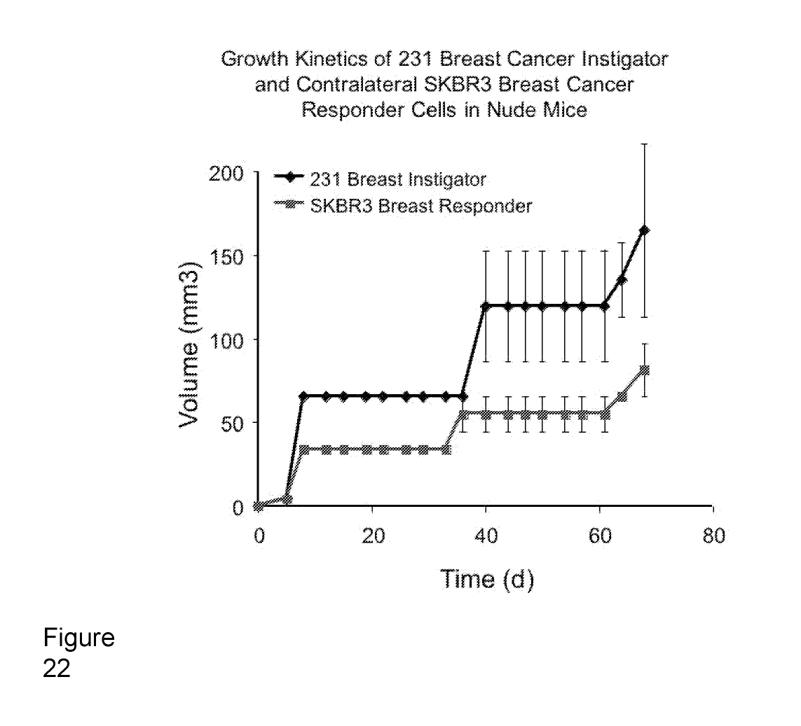

Here, we applied the SKBR3 cell line to the systemic instigation protocol and found that instigating tumors can enhance the growth of SKBR3 tumors. When implanted contralaterally to Matrigel, the Her2+SKBR3 cells formed small masses after a lag period of 20 days; however, these masses shrunk and resolved by the experimental endpoint (FIG. 21). In contrast, the SKBR3 cells that were implanted contralaterally to MDA-MB-231 instigating tumors formed vigorously growing tumors after a lag period of only ˜7 days; these tumors attained a final volume of ˜80 mm³ (FIG. 21). In fact, the SKBR3 tumors closely mirrored the growth kinetics of the contralateral instigating tumors (FIG. 22).

Such an experimental system now allows us to study xenografted growth of Her2+ cell lines in a way that we have not been able to do in the past. With this system, it may be possible to study the response of these Her2+ tumors to anti-Her2 therapy and study mechanisms of tumor resistance. This may also allow us to study use of new drugs, etc to target Her 2.

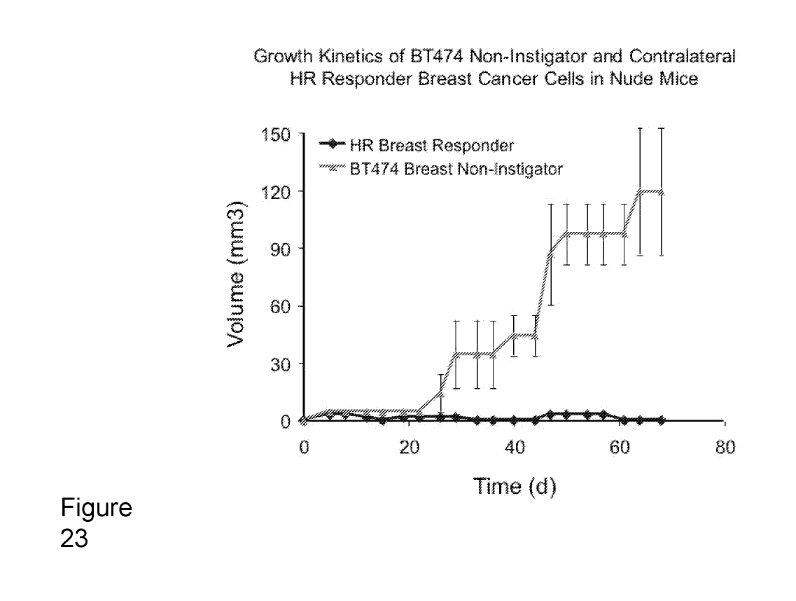

We also found that the Her2+BT474 cell line grows efficiently in host Nude mice; yet, despite its vigorous growth, this tumor cell line is unable to instigate the growth of contralateral responding HMLER-HR breast cancer cells (FIG. 23). Thus, the BT474 cells are considered non-instigators in our experimental model. The discovery of a non-instigating breast cancer cell line is important for the following reasons: i) we can compare instigating and non-instigating breast cancer cells for potential identification of instigating and/or inhibitory molecular mechanisms (i.e. cytokine profiles); ii) we can analyze the bone marrow of these tumor-bearing mice (as described by previous embodiments).

### REFERENCES

Adwan, H., Bauerle, T. J., and Berger, M. R. (2004). Downregulation of osteopontin and bone sialoprotein II is related to reduced colony formation and metastasis formation of MDA-MB-231 human breast cancer cells. Cancer Gene Ther 11, 109-120.
Ariztia, E. V., Subbarao, V., Solt, D. B., Rademaker, A. W., Iyer, A. P., and Oltvai, Z. N. (2003). Osteopontin contributes to hepatocyte growth factor-induced tumor growth and metastasis formation. Exp Cell Res 288, 257-267.
Bhowmick, N. A., Neilson, E. G., and Moses, H. L. (2004). Stromal fibroblasts in cancer initiation and progression. Nature 432, 332-337.
Camphausen, K., Moses, M. A., Beecken, W. D., Khan, M. K., Folkman, J., and O'Reilly, M. S. (2001). Radiation therapy to a primary tumor accelerates metastatic growth in mice. Cancer Res 61, 2207-2211.
Chatterjee, S. K., and Zetter, B. R. (2005). Cancer biomarkers: knowing the present and predicting the future. Future Oncol 1, 37-50.
Cook, A. C., Tuck, A. B., McCarthy, S., Turner, J. G., Irby, R. B., Bloom, G. C., Yeatman, T. J., and Chambers, A. F. (2005). Osteopontin induces multiple changes in gene expression that reflect the six “hallmarks of cancer” in a model of breast cancer progression. Mol Carcinog 43, 225-236.
Coussens, L. M., and Werb, Z. (2002). Inflammation and cancer. Nature 420, 860-867.
De Palma, M., Venneri, M. A., Galli, R., Sergi, L. S., Politi, L. S., Sampaolesi, M., and Naldini, L. (2005). Tie2 identifies a hematopoietic lineage of proangiogenic monocytes required for tumor vessel formation and a mesenchymal population of pericyte progenitors. Cancer Cell 8, 211-226.
Direkze, N. C., and Alison, M. R. (2006). Bone marrow and tumour stroma: an intimate relationship. Hematol Oncol.
Elenbaas, B., Spirio, L., Koerner, F., Fleming, M. D., Zimonjic, D. B., Donaher, J. L., Popescu, N. C., Hahn, W. C., and Weinberg, R. A. (2001). Human breast cancer cells generated by oncogenic transformation of primary mammary epithelial cells. Genes Dev 15, 50-65.
Elenbaas, B., and Weinberg, R. A. (2001). Heterotypic signaling between epithelial tumor cells and fibroblasts in carcinoma formation. Exp Cell Res 264, 169-184.
Feng, F., and Rittling, S. R. (2000). Mammary tumor development in MMTV-c-myc/MMTV-v-Ha-ras transgenic mice is unaffected by osteopontin deficiency. Breast Cancer Res Treat 63, 71-79.
Furger, K. A., Menon, R. K., Tuck, A. B., Bramwell, V. H., and Chambers, A. F. (2001). The functional and clinical roles of osteopontin in cancer and metastasis. Curr Mol Med 1, 621-632.
Gohongi, T., Fukumura, D., Boucher, Y., Yun, C. O., Soff, G. A., Compton, C., Todoroki, T., and Jain, R. K. (1999). Tumor-host interactions in the gallbladder suppress distal angiogenesis and tumor growth: involvement of transforming growth factor beta1. Nat Med 5, 1203-1208.
Graudens, E., Boulanger, V., Mollard, C., Mariage-Samson, R., Barlet, X., Gremy, G., Couillault, C., Lajemi, M., Piatier-Tonneau, D., Zaborski, P., et al. (2006). Deciphering cellular states of innate tumor drug responses. Genome Biol 7, R19.
Hahn, W. C., Counter, C. M., Lundberg, A. S., Beijersbergen, R. L., Brooks, M. W., and Weinberg, R. A. (1999). Creation of human tumour cells with defined genetic elements. Nature 400, 464-468.
Hayashi, C., Rittling, S., Hayata, T., Amagasa, T., Denhardt, D., Ezura, Y., Nakashima, K., and Noda, M. (2007). Serum osteopontin, an enhancer of tumor metastasis to bone, promotes B16 melanoma cell migration. J Cell Biochem.
Hiratsuka, S., Nakamura, K., Iwai, S., Murakami, M., Itoh, T., Kijima, H., Shipley, J. M., Senior, R. M., and Shibuya, M. (2002). MMP9 induction by vascular endothelial growth factor receptor-1 is involved in lung-specific metastasis. Cancer Cell 2, 289-300.
Hiratsuka, S., Watanabe, A., Aburatani, H., and Maru, Y. (2006). Tumour-mediated upregulation of chemoattractants and recruitment of myeloid cells predetermines lung metastasis. Nat Cell Biol 8, 1369-1375.
Hylander, B. L., Pitoniak, R., Penetrante, R. B., Gibbs, J. F., Oktay, D., Cheng, J., and Repasky, E. A. (2005). The anti-tumor effect of Apo2L/TRAIL on patient pancreatic adenocarcinomas grown as xenografts in SCID mice. J Transl Med 3, 22.
Ince, T. A., Richardson, A. L., Bell, G. W., Saitoh, M., Godar, S., Karnoub, A. E., Iglehart, J. D., and Weinberg, R. A. (2007). Transformation of different human breast epithelial cell types leads to distinct tumor phenotypes. Cancer Cell 12, 160-170.
Ishii, G., Sangai, T., Oda, T., Aoyagi, Y., Hasebe, T., Kanomata, N., Endoh, Y., Okumura, C., Okuhara, Y., Magae, J., et al. (2003). Bone-marrow-derived myofibroblasts contribute to the cancer-induced stromal reaction. Biochem Biophys Res Commun 309, 232-240.
Iwata, M., Awaya, N., Graf, L., Kahl, C., and Torok-Storb, B. (2004). Human marrow stromal cells activate monocytes to secrete osteopontin, which down-regulates Notch1 gene expression in CD34+ cells. Blood 103, 4496-4502.
Kang, Y., Siegel, P. M., Shu, W., Drobnjak, M., Kakonen, S. M., Cordon-Cardo, C., Guise, T. A., and Massague, J. (2003). A multigenic program mediating breast cancer metastasis to bone. Cancer Cell 3, 537-549.
Kaplan, R. N., Riba, R. D., Zacharoulis, S., Bramley, A. H., Vincent, L., Costa, C., MacDonald, D. D., Jin, D. K., Shido, K., Kerns, S. A., et al. (2005). VEGFR1-positive haematopoietic bone marrow progenitors initiate the pre-metastatic niche. Nature 438, 820-827.
Klarman, K., Ortiz, M., Davies, M., and Keller, J. R. (2003). Identification of in vitro growth conditions for c-Kit-negative hematopoietic stem cells. Blood 102, 3120-3128.
Klein, C. A. (2004). Gene expression sigantures, cancer cell evolution and metastatic progression. Cell Cycle 3, 29-31.
Kondo, M., Wagers, A. J., Manz, M. G., Prohaska, S. S., Scherer, D. C., Beilhack, G. F., Shizuru, J. A., and Weissman, I. L. (2003). Biology of hematopoietic stem cells and progenitors: implications for clinical application. Annu Rev Immunol 21, 759-806.
Kopp, H. G., Ramos, C. A., and Rafii, S. (2006). Contribution of endothelial progenitors and proangiogenic hematopoietic cells to vascularization of tumor and ischemic tissue. Curr Opin Hematol 13, 175-181.
Kotton, D. N., Summer, R. S., Sun, X., Ma, B. Y., and Fine, A. (2003). Stem cell antigen-1 expression in the pulmonary vascular endothelium. Am J Physiol Lung Cell Mol Physiol 284, L990-996.
Lamagna, C., and Bergers, G. (2006). The bone marrow constitutes a reservoir of pericyte progenitors. J Leukoc Biol 80, 677-681.
Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436, 518-524.
Mor, G., Visintin, I., Lai, Y., Zhao, H., Schwartz, P., Rutherford, T., Yue, L., Bray-Ward, P., and Ward, D. C. (2005). Serum protein markers for early detection of ovarian cancer. Proc Natl Acad Sci USA 102, 7677-7682.
Murdoch, C., Giannoudis, A., and Lewis, C. E. (2004). Mechanisms regulating the recruitment of macrophages into hypoxic areas of tumors and other ischemic tissues. Blood 104, 2224-2234.
Naka, T., Sugamura, K., Hylander, B. L., Widmer, M. B., Rustum, Y. M., and Repasky, E. A. (2002). Effects of tumor necrosis factor-related apoptosis-inducing ligand alone and in combination with chemotherapeutic agents on patients' colon tumors grown in SCID mice. Cancer Res 62, 5800-5806.
Naume, B., Wiedswang, G., Borgen, E., Kvalheim, G., Karesen, R., Qvist, H., Janbu, J., Harbitz, T., and Nesland, J. M. (2004). The prognostic value of isolated tumor cells in bone marrow in breast cancer patients: evaluation of morphological categories and the number of clinically significant cells. Clin Cancer Res 10, 3091-3097.
Nilsson, S. K., Johnston, H. M., Whitty, G. A., Williams, B., Webb, R. J., Denhardt, D. T., Bertoncello, I., Bendall, L. J., Simmons, P. J., and Haylock, D. N. (2005). Osteopontin, a key component of the hematopoietic stem cell niche and regulator of primitive hematopoietic progenitor cells. Blood 106, 1232-1239.
O'Regan, A., and Fleming, C. (2002). Osteopontin as a biomarker for ovarian cancer. Jama 287, 3208-3209; author reply 3209-3210.
O'Reilly, M. S., Boehm, T., Shing, Y., Fukai, N., Vasios, G., Lane, W. S., Flynn, E., Birkhead, J. R., Olsen, B. R., and Folkman, J. (1997). Endostatin: an endogenous inhibitor of angiogenesis and tumor growth. Cell 88, 277-285.
O'Reilly, M. S., Holmgren, L., Shing, Y., Chen, C., Rosenthal, R. A., Moses, M., Lane, W. S., Cao, Y., Sage, E. H., and Folkman, J. (1994). Angiostatin: a novel angiogenesis inhibitor that mediates the suppression of metastases by a Lewis lung carcinoma. Cell 79, 315-328.
Oft, M., Peli, J., Rudaz, C., Schwarz, H., Beug, H., and Reichmann, E. (1996). TGF-beta1 and Ha-Ras collaborate in modulating the phenotypic plasticity and invasiveness of epithelial tumor cells. Genes Dev 10, 2462-2477.
Olumi, A. F., Grossfeld, G. D., Hayward, S. W., Carroll, P. R., Tlsty, T. D., and Cunha, G. R. (1999). Carcinoma-associated fibroblasts direct tumor progression of initiated human prostatic epithelium. Cancer Res 59, 5002-5011.
Orimo, A., Gupta, P. B., Sgroi, D. C., Arenzana-Seisdedos, F., Delaunay, T., Naeem, R., Carey, V. J., Richardson, A. L., and Weinberg, R. A. (2005). Stromal fibroblasts present in invasive human breast carcinomas promote tumor growth and angiogenesis through elevated SDF-1/CXCL12 secretion. Cell 121, 335-348.
Pollard, J. W. (2004). Tumour-educated macrophages promote tumour progression and metastasis. Nat Rev Cancer 4, 71-78.
Ramankulov, A., Lein, M., Kristiansen, G., Meyer, H. A., Loening, S. A., and Jung, K. (2007). Elevated plasma osteopontin as marker for distant metastases and poor survival in patients with renal cell carcinoma. J Cancer Res Clin Oncol 133, 643-652.
Randall, T. D. and Weissman, I. L. (1997). Phenotypic and functional changes induced at the clonal level in hematopoietic stem cells after 5-fluorouracil treatment. Blood 89, 3596-3606.
Randall, T. D. and Weissman, I. L. (1998). Characterization of a population of cells in the bone marrow that phenotypically mimics hematopoietic stem cells: resting stem cells or mystery population? Stem Cells 16, 38-48.
Richardson, A. L., Wang, Z. C., De Nicolo, A., Lu, X., Brown, M., Miron, A., Liao, X., Iglehart, J. D., Livingston, D. M., and Ganesan, S. (2006). X chromosomal abnormalities in basal-like human breast cancer. Cancer Cell 9, 121-132.
Rittling, S. R., and Chambers, A. F. (2004). Role of osteopontin in tumour progression. Br J Cancer 90, 1877-1881.
Rossi, D. J., Bryder, D., Seita, J., Nussenzweig, A., Hoeijmakers, J., and Weissman, I. L. (2007). Deficiencies in DNA damage repair limit the function of haematopoietic stem cells with age. Nature 447, 725-729.
Rudland, P. S., Platt-Higgins, A., El-Tanani, M., De Silva Rudland, S., Barraclough, R., Winstanley, J. H., Howitt, R., and West, C. R. (2002). Prognostic significance of the metastasis-associated protein osteopontin in human breast cancer. Cancer Res 62, 3417-3427.
Scadden, D. T. (2006). The stem-cell niche as an entity of action. Nature 441, 1075-1079.
Schmits, R., Filmus, J., Gerwin, N., Senaldi, G., Kiefer, F., Kundig, T., Wakeham, A., Shahinian, A., Catzavelos, C., Rak, J., et al. (1997). CD44 regulates hematopoietic progenitor distribution, granuloma formation, and tumorigenicity. Blood 90, 2217-2233.
Scott, L. M., Priestley, G. V., and Papayannopoulou, T. (2003). Deletion of alpha4 integrins from adult hematopoietic cells reveals roles in homeostasis, regeneration, and homing. Mol Cell Biol 23, 9349-9360.
Sendobry, S. M., Cornicelli, J. A., Welch, K., Bocan, T., Tait, B., Trivedi, B. K., Colbry, N., Dyer, R. D., Feinmark, S. J., and Daugherty, A. (1997). Attenuation of diet-induced atherosclerosis in rabbits with a highly selective 15-lipoxygenase inhibitor lacking significant antioxidant properties. Br J Pharmacol 120, 1199-1206.
Shojaei, F., Wu, X., Malik, A. K., Zhong, C., Baldwin, M. E., Schanz, S., Fuh, G., Gerber, H. P., and Ferrara, N. (2007). Tumor refractoriness to anti-VEGF treatment is mediated by CD11b+Grl+myeloid cells. Nat Biotechnol 25, 911-920.
Singhal, H., Bautista, D. S., Tonkin, K. S., O'Malley, F. P., Tuck, A. B., Chambers, A. F., and Harris, J. F. (1997). Elevated plasma osteopontin in metastatic breast cancer associated with increased tumor burden and decreased survival. Clin Cancer Res 3, 605-611.
Song, S., Ewald, A. J., Stallcup, W., Werb, Z., and Bergers, G. (2005). PDGFRbeta+perivascular progenitor cells in tumours regulate pericyte differentiation and vascular survival. Nat Cell Biol 7, 870-879.
Stewart, S. A., Dykxhoorn, D. M., Palliser, D., Mizuno, H., Yu, E. Y., An, D. S., Sabatini, D. M., Chen, I. S., Hahn, W. C., Sharp, P. A., et al. (2003). Lentivirus-delivered stable gene silencing by RNAi in primary cells. Rna 9, 493-501.
Stier, S., Ko, Y., Forkert, R., Lutz, C., Neuhaus, T., Grunewald, E., Cheng, T., Dombkowski, D., Calvi, L. M., Rittling, S. R., and Scadden, D. T. (2005). Osteopontin is a hematopoietic stem cell niche component that negatively regulates stem cell pool size. J Exp Med 201, 1781-1791.
Tlsty, T. D. (2001). Stromal cells can contribute oncogenic signals. Semin Cancer Biol 11, 97-104.
Tuck, A. B., and Chambers, A. F. (2001). The role of osteopontin in breast cancer: clinical and experimental studies. J Mammary Gland Biol Neoplasia 6, 419-429.
Udagawa, T., Puder, M., Wood, M., Schaefer, B. C., and D'Amato, R. J. (2006). Analysis of tumor-associated stromal cells using SCID GFP transgenic mice: contribution of local and bone marrow-derived host cells. Faseb J 20, 95-102.
van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.
Wai, P. Y., Mi, Z., Guo, H., Sarraf-Yazdi, S., Gao, C., Wei, J., Marroquin, C. E., Clary, B., and Kuo, P. C. (2005). Osteopontin silencing by small interfering RNA suppresses in vitro and in vivo CT26 murine colon adenocarcinoma metastasis. Carcinogenesis 26, 741-751.
Yang, L., DeBusk, L. M., Fukuda, K., Fingleton, B., Green-Jarvis, B., Shyr, Y., Matrisian, L. M., Carbone, D. P., and Lin, P. C. (2004). Expansion of myeloid immune suppressor Gr⁺ CD11b+ cells in tumor-bearing host directly promotes tumor angiogenesis. Cancer Cell 6, 409-421.
Yu, Y. P., Landsittel, D., Jing, L., Nelson, J., Ren, B., Liu, L., McDonald, C., Thomas, R., Dhir, R., Finkelstein, S., et al. (2004). Gene expression alterations in prostate cancer predicting tumor aggression and preceding development of malignancy. J Clin Oncol 22, 2790-2799.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

### INCORPORATION OF REFERENCES

All publications and patent documents cited in this application are incorporated by reference in their entirety to the same extent as if the contents of each individual publication or patent document were incorporated herein.

We claim:

1. A method for studying tumor outgrowth or metastasis, the method comprising the steps of:
(a) providing an animal host;
(b) introducing into the animal host one or more cells that instigate the growth of an otherwise indolent tumor;
(c) introducing into the animal host a tumor; wherein the presence of the one or more cells or a progeny thereof in the animal host enhances the growth and/or metastasis of the tumor.

2. The method of claim 1, wherein the tumor is otherwise indolent in the animal host.

3. The method of claim 1, wherein the tumor is a human tumor.

4.-9. (canceled)

10. The method of claim 1, wherein the one or more cells that instigate the growth of an otherwise indolent tumor comprise tumor cells.

11.-15. (canceled)

16. The method of claim 1, wherein the one or more cells that instigate the growth of an otherwise indolent tumor comprise genetically modified cells.

17.-25. (canceled)

26. The method of claim 1 further comprising a step of mixing the one or more cells with the tumor before introducing into the animal host.

27.-30. (canceled)

31. The method of claim 1 further comprising administering a test agent to the animal host.

32. The method of claim 31 further comprising evaluating the ability of the test agent to inhibit the growth or metastasis of the tumor.

33.-41. (canceled)

42. A method for studying tumor outgrowth or metastasis, the method comprising the steps of:
(a) providing an activated animal host, wherein the activated animal host enhances the growth or metastasis of an indolent tumor compared to a corresponding un-activated animal host; and
(b) introducing a tumor to the activated animal host.

43. The method of claim 42, wherein the tumor is a human tumor.

44.-76. (canceled)

77. A method for evaluating the ability of an agent to inhibit tumor outgrowth or metastasis, the method comprising the steps of:
(a) providing an animal host that instigates the growth or metastasis of an otherwise indolent tumor; and
(b) introducing a tumor into the animal host;
(c) administering an agent to the animal host bearing the tumor; and
(d) evaluating the ability of the agent to inhibit the growth or metastasis of the tumor.

78. (canceled)

79. The method of claim 77, wherein the animal host bears a first tumor that enhances the growth of the otherwise indolent tumor.

80.-93. (canceled)

94. A method comprising steps of:
(a) providing a sample obtained from an animal host that instigates the growth of an otherwise indolent tumor;
(b) providing a control sample; and
(c) comparing the sample of (a) with the control sample of (b) so as to identify one or more components that differ between the samples, wherein a component that differs between the two samples is identified as a candidate modulator of systemic tumor instigation.

95. The method of claim 94, wherein the animal host of step (a) bears an instigator.

96. The method of claim 95, wherein the instigator is selected from the group consisting of one or more cells, an implanted tumor sample, a spontaneously-arising tumor, a surgical wound, and combinations thereof.

97.-109. (canceled)

110. The method of claim 94, wherein the sample of step (a) comprises activated bone marrow cells obtained from the animal host.

111.-125. (canceled)

126. The method of claim 94, wherein step (a) comprises
providing a sample obtained from an animal host bearing one or more cells or compositions secreting or releasing OPN.

127. The method of claim 126, further comprising administering the identified candidate modulator to an animal host bearing an indolent tumor and determining whether the identified candidate modulator promotes outgrowth or metastasis of the indolent tumor.

128.-140. (canceled)

141. The method of claim 126, further comprising a step of testing if the candidate modulator is a modulator of tumor metastasis or outgrowth.

142.-143. (canceled)

144. The method of claim 141, wherein the testing step comprising administering the candidate modulator to an animal host bearing an indolent tumor and determining whether the candidate modulator promotes outgrowth or metastasis of the indolent tumor.

145. (canceled)

146. A method of identifying a target for development of an anti-tumor therapy comprising:
(a) identifying a modulator of systemic instigation thereby identifying a target for development of an anti-tumor therapy or (b) identifying a modulator of systemic instigation; and identifying a receptor, cell surface molecule, or cell type to which the modulator binds, wherein the receptor, cell surface molecule, or cell type is identified as a target for development of an anti-tumor therapy.

147.-153. (canceled)

154.-172. (canceled)

173. A method of testing a candidate anti-tumor agent, the method comprising:
(a) administering a candidate anti-tumor agent to an animal host bearing one or more cells that instigate the growth of an otherwise indolent tumor; and
(b) determining whether the candidate anti-tumor agent increases or decreases the population of a cell type that enhances outgrowth or metastasis of a tumor or inhibits migration of said cell type to a tumor.

174.-222. (canceled)
